(19) United States
(12) Patent Application Publication
(10) Pub. No.: US 2023/0162815 A1
(43) Pub. Date: May 25, 2023

(54) METHODS AND SYSTEMS FOR ACCURATE GENOTYPING OF REPEAT POLYMORPHISMS

(71) Applicant: Tempus Labs, Inc., Chicago, IL (US)

(72) Inventors: Gene Selkov, Chicago, IL (US); Kurt Oliver Gaastra, Hamilton (NZ); Sean Allistair Irvine, Hamilton (NZ); Leonard Eric Trigg, Hamilton (NZ); Francisco Miguel De La Vega, San Mateo, CA (US)

(21) Appl. No.: 17/981,212

(22) Filed: Nov. 4, 2022

Related U.S. Application Data
(60) Provisional application No. 63/281,474, filed on Nov. 19, 2021.

(51) Int. Cl. G16B 20/20; G16B 30/10; C12Q 1/6827

(52) U.S. Cl. CPC G16B 20/20; G16B 30/10; C12Q 1/6827; C12Q 2600/156

(57) ABSTRACT

Methods, systems, and software are provided for determining a genotype for a genomic locus comprising a tandem repeat having contiguous repeat units. Sequence reads that encompass and map to the tandem repeat are obtained. A repeat count distribution for the number of repeat units in the reads is determined. Sets of adjustment factors are obtained, each set (i) corresponding to a different allele having a different repeat unit count and (ii) including corresponding adjustment factors for a range of repeat unit counts. Candidate genotypes correspond to combinations of two alleles in a plurality of candidate alleles. Each candidate genotype is assigned a likelihood based at least in part on, for each allele in the candidate genotype: (i) a proportion of sequence reads having the repeat count corresponding to the allele and (ii) an adjustment factor from the corresponding set. The candidate genotype having the highest likelihood is selected.

### CROSS-REFERENCE TO RELATED APPLICATIONS

This application claims the benefit of U.S. Provisional Application No. 63/281,474, filed on Nov. 19, 2021, which is expressly incorporated by reference in its entirety for all purposes.

### TECHNICAL FIELD

The present disclosure relates generally to genotyping repeat polymorphisms using sequence reads.

### BACKGROUND

Precision oncology is the practice of tailoring cancer therapy to the unique genomic, epigenetic, and/or transcriptomic profile of an individual patient or tumor. This is in contrast to conventional methods for treating a cancer patient based merely on the type of cancer the patient is afflicted with, e.g., treating all breast cancer patients with a first therapy and all lung cancer patients with a second therapy. Precision oncology was borne out of many observations that different patients diagnosed with the same type of cancer responded very differently to common treatment regimes. Over time, researchers have identified genomic, epigenetic, and transcriptomic markers that facilitate some level of prediction as to how an individual patient, or cancer, will respond to a particular treatment modality.

Therapy targeted to specific genomic alterations is already the standard of care in several tumor types (e.g., as described in the National Comprehensive Cancer Network (NCCN) guidelines for melanoma, colorectal cancer, and non-small cell lung cancer). These few, well known mutations in the NCCN guidelines can be addressed with individual assays or small next-generation sequencing (NGS) panels. However, for the largest number of patients to benefit from personalized oncology, molecular alterations that can be targeted with off-label drug indications, combination therapy, or tissue agnostic immunotherapy should be assessed. See Schwaederle et al. 2016 JAMA Oncol. 2, 1452-1459; Schwaederle et al. 2015 J Clin Oncol. 32, 3817-3825; and Wheler et al. 2016 Cancer Res. 76, 3690-3701. Large panel NGS assays also cast a wider net for clinical trial enrollment. See Coyne et al. 2017 Curr. Probl. Cancer 41, 182-193; and Markman 2017 Oncology 31, 158,168.

Genomic analysis is rapidly becoming routine clinical practice to provide tailored patient treatments and improve outcomes. See Fernandes et al. 2017 Clinics 72, 588-594. Indeed, recent studies indicate that clinical care is guided by NGS assay results for 30-40% of patients receiving such testing. See Hirshfield et al. 2016 Oncologist 21, 1315-1325; Groisberg et al. 2017 Oncotarget 8, 39254-39267; Ross et al. JAMA Oncol. 1, 40-49; and Ross et al. 2015 Arch. Pathol. Lab Med. 139, 642-649. There is growing evidence that patients who receive therapeutic advice guided by genetics have better outcomes. See, for example, Wheler et al., in which matching scores (e.g., scores based on the number of therapeutic associations and genomic aberrations per patient) were used to demonstrate that patients with higher matching scores have a greater frequency of stable disease, longer time to treatment failure, and greater overall survival (2016 Cancer Res. 76, 3690-3701). Such methods may be particularly useful for patients who have already failed multiple lines of therapy.

Targeted therapies have shown significant improvements in patient outcomes, especially in terms of progression-free survival. See Radovich et al. 2016 Oncotarget 7, 56491-56500. Further, recent evidence reported from the IMPACT trial found that the three-year overall survival for patients given a molecularly matched therapy was more than twice that of non-matched patients (15% vs. 7%). See Bankhead, “IMPACT Trial: Support for Targeted Cancer Tx Approaches.” MedPageToday. Jun. 5, 2018; and ASCO Post, “2018 ASCO: IMPACT Trial Matches Treatment to Genetic Changes in the Tumor to Improve Survival Across Multiple Cancer conditions.” The ASCO POST. Jun. 6, 2018. Estimates of the proportion of patients for whom genetic testing changes the trajectory of their care vary widely, from approximately 10% to more than 50%. See Fernandes et al. 2017 Clinics 72, 588-594.

### SUMMARY

Given the above background, what is needed in the art are improved ways to identify specific genomic alterations in patients. The present disclosure addresses these and other needs by providing systems and methods for using next-generation sequencing (NGS) technology to identify and characterize genetic variations or polymorphisms for genomic sequences, particularly in those containing short tandem repeats (STR). There is also a need for improved ways to identify and predict patient responses to therapeutic agents, including potential adverse effects. The present disclosure addresses these and other needs by providing systems and methods for using NGS technology to identify and categorize genetic variations or polymorphisms that, in some cases, result in actionable associations. For instance, the determination of individual genotypes brings the ability to not only understand genome-disease associations but also the possibility of better understanding the risk of treatment side effects.

Provided herein are systems and methods for determining a genotype of a subject at a genomic locus comprising a tandem repeat sequence. The systems and methods provided herein are useful for associating particular polymorphisms with a subject's risk of adverse drug reactions, for providing product labels, for developing companion diagnostic tests, and/or as a component of a panel of tests to support an individualized medicine approach. This approach is also advantageous in that it allows for the determination of polymorphic variants in repeats using NGS results, thus reducing the cost, time, and amount of DNA needed to perform analysis compared to other methods. In some embodiments, this approach allows genotyping to be carried out where it is not otherwise possible, and, when used in combination with additional methods (e.g., moderately deep UMI sequencing protocols), can make genotyping more robust and cost-effective. In some instances, this approach is utilized in research settings to elucidate the heterogeneity of therapeutic responses within and among patients, or in the clinical laboratory to potentially guide precision oncology treatments.

For instance, in some embodiments, methods are provided for determining a previously unknown association between a genotype and a therapeutic response. In some embodiments, the method includes accessing a database storing information about, for each respective subject in a plurality of subjects, a corresponding genotype for the subject at one or more genomic loci (e.g., a genomic locus having one or more tandem repeats), a corresponding treatment administered to the respective subject for treatment of a clinical condition, and a corresponding outcome for the treatment of the subject. The method then includes determining an association between one or more treatments administered to subjects with a particular genotype and the clinical outcomes for the subjects based on the data in the database.

One aspect of the present disclosure provides a method for determining a genotype of a subject at a genomic locus comprising a tandem repeat, from a plurality of candidate genotypes for the genomic locus. In some embodiments, the method includes obtaining, in electronic form, a first set of sequence reads obtained from a biological sample of the subject that map to the tandem repeat in the genomic locus, where the tandem repeat consists of a plurality of contiguous nucleotide repeat units and each respective sequence read in the first set of sequence reads encompasses the tandem repeat. In some embodiments, the method further includes determining, for each respective sequence read in the first set of sequence reads, a corresponding repeat count of the number of repeat units in the plurality of contiguous repeat units in the respective sequence read, thereby determining a distribution of repeat counts of the number of repeat units in the first set of sequence reads.

In some embodiments, the method further includes obtaining a plurality of sets of repeat count adjustment factors, where each respective set of repeat count adjustment factors corresponds to a candidate allele in a plurality of candidate alleles, each respective candidate allele in the plurality of candidate alleles has a different corresponding number of repeat units for the plurality of contiguous nucleotide repeat units, each respective set of repeat count adjustment factors includes a corresponding repeat count adjustment factor for each respective number of repeat units in a numerical range of repeat units for the plurality of contiguous nucleotide repeat units, and each combination of two respective candidate alleles in the plurality of candidate alleles corresponds to a respective candidate genotype in the plurality of candidate genotypes.

In some implementations, the method further comprises assigning, for each respective candidate genotype in the plurality of candidate genotypes, a corresponding likelihood for the respective candidate genotype based, at least in part, upon, for each respective candidate allele corresponding to the respective candidate genotype: (i) a proportion of sequence reads in the plurality of sequence reads that have the repeat count of the number of repeat units in the plurality of contiguous repeat units corresponding to the respective candidate allele, and (ii) a repeat count adjustment factor matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles. In some such implementations, the method further comprises selecting the respective candidate genotype in the plurality of candidate genotypes having the highest corresponding likelihood.

Another aspect of the present disclosure provides a computer system having one or more processors, and memory storing one or more programs for execution by the one or more processors, the one or more programs comprising instructions for performing any of the methods and/or embodiments disclosed above.

Another aspect of the present disclosure provides a non-transitory computer readable storage medium storing one or more programs configured for execution by a computer, the one or more programs comprising instructions for carrying out any of the methods disclosed above.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIGS. 1A and 1B collectively illustrate a block diagram of an example of a computing device for determining a genotype of a subject at a genomic locus comprising a tandem repeat, in accordance with some embodiments of the present disclosure.

FIG. 2 illustrates an example of a distributed diagnostic environment for determining a genotype of a subject at a genomic locus comprising a tandem repeat, in accordance with some embodiments of the present disclosure.

FIGS. 3A, 3B, 3C, 3D and 3E collectively illustrate an example workflow for determining a genotype of a subject at a genomic locus comprising a tandem repeat, in which optional steps are indicated by dashed boxes, in accordance with some embodiments of the present disclosure.

FIGS. 4A, 4B, and 4C provide example workflows of methods for determining a genotype of a subject at a genomic locus comprising a tandem repeat, in accordance with some embodiments of the present disclosure.

FIGS. 5A, 5B, 5C, 5D, 5E, and 5F are graphic representations of the realignment of sequence reads to candidate alleles representing different possible numbers of repeat units in a repeat sequence of interest, in accordance with an embodiment of the present disclosure.

FIG. 6 illustrates coverage of targeted variant positions in a targeted panel next-generation sequencing (NGS) assay in samples containing interferent substances, in accordance with an embodiment of the present disclosure.

FIG. 7 provides example counts of repeat-spanning sequence reads aligned to multiple linear models of tandem repeat polymorphisms, for an exemplary homozygous genotype of 8 repeat units, in accordance with an embodiment of the present disclosure. Linear models represent “expected” candidate alleles of varying tandem repeat lengths (e.g., expected in a reference population, such as a population of known reference genomes and/or a reference population of subjects). Counts are analyzed using a Bayesian model to produce posterior probabilities and final genotype calls with one or more quality metrics.

FIG. 8 provides example counts of repeat-spanning sequence reads aligned to multiple linear models of tandem repeat polymorphisms, for an exemplary heterozygous genotype of 7 repeat units/8 repeat units, in accordance with an embodiment of the present disclosure. Linear models represent “expected” candidate alleles of varying tandem repeat lengths (e.g., expected in a reference population, such as a population of known reference genomes and/or a reference population of subjects). Counts are analyzed using a Bayesian model to produce posterior probabilities and final genotype calls with one or more quality metrics.

FIG. 9 is a graphical representation of the distribution of repeat lengths in read alignments from samples including repeat lengths of 6, 7, 8, and 9 repeat units, in accordance with an embodiment of the present disclosure. “Empirical” refers to distributions observed in real samples, and “model” is the mathematical model fit that can be used to provide an error model for Bayesian genotyping.

FIG. 10 is a receiver-operator curve showing the discriminating ability of two types of quality scores for genotype calls, in accordance with an embodiment of the present disclosure. Read depth (DP) is shown in black, and Phred-scaled genotype quality (GQ) is shown in gray.

FIGS. 11A, 11B, and 11C collectively illustrate reporting a genotype of a subject at a genomic locus comprising a tandem repeat, in accordance with some embodiments of the present disclosure.

FIG. 12 illustrates coverage of targeted variant positions in a targeted panel next-generation sequencing (NGS) assay in samples obtained from reference specimens, in accordance with an embodiment of the present disclosure.

FIG. 13 illustrates coverage of targeted variant positions in a targeted panel next-generation sequencing (NGS) assay in samples obtained from clinical specimens, in accordance with an embodiment of the present disclosure.

FIG. 14 illustrates coverage of targeted variant positions in a targeted panel next-generation sequencing (NGS) assay in samples titrated at a plurality of concentrations, in accordance with an embodiment of the present disclosure.

FIGS. 15A and 15B illustrate coverage of targeted variant positions in a targeted panel next-generation sequencing (NGS) assay performed across multiple replicates within a sequencing run and across multiple sequencing runs, in accordance with an embodiment of the present disclosure.

FIG. 16 illustrates coverage of targeted variant positions in a targeted panel next-generation sequencing (NGS) assay performed on a plurality of sequencing devices, in accordance with an embodiment of the present disclosure.

Like reference numerals refer to corresponding parts throughout the several views of the drawings.

### DETAILED DESCRIPTION

### A. Introduction

A major contributor to the availability of individualized medicine is the technology for high-throughput sequencing of DNA. The determination of individual genotypes brings the ability to not only understand genome-disease associations but also the possibility of better understanding the risk of disease treatment side effects, when a genome-side effect association is established (see, e.g., Nygen et al., Nature Comm., 10: 1579 (2019)). Known in general as pharmacogenomics, the goal of this area of study is to elucidate the genetic factors that influence drug responses. This area is now utilizing next-generation sequencing (NGS) technology to further identify and categorize genetic variations or polymorphisms that can result in actionable associations. These associations are often practical, and, after being characterized, an actionable association can be used on product labels (e.g., to provide more accurate toxicity risk information), as a driver for companion diagnostic tests, and/or as a component of specialized tests to support an individualized medicine approach (see, e.g., Russell et al., Drug Metabolism Rev., 53(2): 253-78 (2021)).

One focus of pharmacogenomic research is the treatment of cancer, where the response to many drugs has been found to have a genetic basis, often in the genes that encode the enzymes involved in the metabolism of the drugs within the body (see, e.g., Miteva-Marcheva et al., Biomarker Rev., 8:33 (2020)). This focus is driven by a generally narrow therapeutic index for chemotherapeutic drugs—that is, a narrow concentration range between effective treatment and unacceptable side effects—and the high risk of those unacceptable side effects being life-threatening. Some patient genotypes that have been found of interest include those of dihydropyrimidine dehydrogenase (DPD), thymidine synthetase (TS), methylene tetrahydrofolate reductase (MTHFR), thiopurine S-methyltransferase (TPMT), uridine diphosphate glucuronosyl transferase (UGT), glutathione S-transferases (GSTs), excision repair cross complementing groups 1 and 2 (ERCC 1 and 2), ATP binding cassettes (ABCB1, ABCC2, and ABCG2) and X-ray cross complementing group 1 (XRCC1). There are many other significant gene-drug interaction associations yet to be discovered just in the cancer therapeutic area.

One subset of polymorphisms that can impact these genotypes is the presence or absence of short tandem repeats (STR). For example, in some cases, variations in the number of repeats present in a genomic area known to include STR sequences has proven to impact gene expression of associated coding regions (see, e.g., Fotsing et al., Nature Genetics, 51:1652-59 (2019)). One such repeat polymorphism associated with impact on chemotherapeutic metabolism is the UGT1A1*28 promoter polymorphism which impacts the expression of the UGT1A1 gene (see, e.g., Iyer et al., Pharmacogen. J., 2:43-7 (2002)). However, the use of NGS to identify and elucidate such genomic regions is hampered by many technological challenges. These include difficulties in accurate variant calling for such short repeat lengths (see, e.g., Bahlo et al., F1000Res., 7:F1000 Faculty Rev—736 (2018)) and inaccurate reads that result from DNA polymerase stuttering in the polymerase chain reaction (PCR) amplification phase (see, e.g., Raz et al., Nucl. Acid. Res., 47(5):2436-45 (2019)). This has resulted, in many cases, in the reliance on older, more labor intensive, and more expensive technologies for these kinds of analyses, such as microarrays, real time PCR, primer extension with time of flight (TOF) mass spectrometry, or restriction fragment length polymorphism (RFLP) fragment analysis in capillary electrophoresis.

In many instances, NGS is increasingly the technology of choice to detect and report genetic variants of clinical relevance due to its cost effectiveness and throughput, and many clinical tests that detect single-nucleotide or small insertion/deletion variants rely on this technology. The use of a second technology platform solely for determining polymorphic variants in repeats creates considerable inconvenience. In particular, this adds cost, time, and labor, and further requires additional specimen DNA on which the secondary analysis is performed. It is therefore advantageous to utilize existing NGS results to perform polymorphism analysis for STR regions.

As indicated above, and as evident from these teachings, there remains a need in the art for systems and methods for accurate detection of the precise genotype of polymorphisms present in repeat-containing genomic sequences, using next-generation sequencing. These systems and methods, as well as other uses and embodiments of any output thereof, such as associating particular polymorphisms with drug reactions and/or developing companion diagnostic tests, are provided by the present disclosure.

In some embodiments, the present disclosure provides systems and methods for determining a genotype of a subject at a genomic locus comprising a tandem repeat, to improve treatment predictions, outcomes, and risk assessments.

For instance, in one aspect, the disclosure provides systems and methods for determining a genotype of a subject at a genomic locus comprising a tandem repeat, from a plurality of candidate genotypes for the genomic locus. In some embodiments, methods include obtaining, in electronic form, a first set of sequence reads obtained from a biological sample of the subject that map to the tandem repeat in the genomic locus, where the tandem repeat consists of a plurality of contiguous nucleotide repeat units and each respective sequence read in the first set of sequence reads encompasses the tandem repeat. In some embodiments, methods further include determining, for each respective sequence read in the first set of sequence reads, a corresponding repeat count of the number of repeat units in the plurality of contiguous repeat units in the respective sequence read, thereby determining a distribution of repeat counts of the number of repeat units in the first set of sequence reads.

In some embodiments, methods further include obtaining a plurality of sets of repeat count adjustment factors, where each respective set of repeat count adjustment factors corresponds to a candidate allele in a plurality of candidate alleles, each respective candidate allele in the plurality of candidate alleles has a different corresponding number of repeat units for the plurality of contiguous nucleotide repeat units, each respective set of repeat count adjustment factors includes a corresponding repeat count adjustment factor for each respective number of repeat units in a numerical range of repeat units for the plurality of contiguous nucleotide repeat units, and each combination of two respective candidate alleles in the plurality of candidate alleles corresponds to a respective candidate genotype in the plurality of candidate genotypes.

In some implementations, methods further include assigning, for each respective candidate genotype in the plurality of candidate genotypes, a corresponding likelihood for the respective candidate genotype based, at least in part, upon, for each respective candidate allele corresponding to the respective candidate genotype: (i) a proportion of sequence reads in the plurality of sequence reads that have the repeat count of the number of repeat units in the plurality of contiguous repeat units corresponding to the respective candidate allele, and (ii) a repeat count adjustment factor matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles. In some such implementations, methods further comprise selecting the respective candidate genotype in the plurality of candidate genotypes having the highest corresponding likelihood.

Benefit

Advantageously, in some embodiments, the systems and methods of the present disclosure are performed using sequence reads obtained from next-generation sequencing (NGS). Particularly, in some embodiments, the genotype for the tandem repeat is determined using the same NGS data used for detecting single-nucleotide (e.g., SNVs) or small insertion/deletion (e.g., indels) variants in companion clinical tests. In some such embodiments, this approach reduces the cost, time, and labor that would otherwise be needed to perform multiple separate sequencing technologies for genotyping repeat polymorphisms and determining single-nucleotide variants or other indels. Moreover, in some such implementations, this approach does not require the collection of additional patient samples for secondary analysis, thus reducing the need for further invasive surgical procedures, in-office visits, and other logistical obstacles.

The systems and methods of the present disclosure provide further benefits in that such approaches are useful in combination with other variant detection assays, research objectives, and/or clinical applications. For instance, in some embodiments, a genotype for a tandem repeat in a genomic sequence is determined using the systems and methods provided herein. In some implementations, the tandem repeat genotype is used to validate a finding from a prior analysis. Alternatively or additionally, in some implementations, the tandem repeat genotype is combined with one or more detected alternative variants (e.g., single-nucleotide variants and/or indels) to form a combined molecular signature that informs a particular downstream application. In some instances, non-limiting examples of downstream applications include, but are not limited to, determination of risk of drug reactions; recommendation of a particular therapeutic drug or dosage thereof; recommendation of a modification or cessation of a particular therapeutic drug or dosage thereof; enrollment in a clinical trial; and/or performing one or more companion assays for an individualized medicine approach. Other downstream applications can include any of the applications set forth below.

As such, in some implementations, the presently disclosed systems and methods provide repeat polymorphism genotyping approaches that can be used to support, validate, and/or bolster a multitude of research and clinical decisions.

In some embodiments, the systems and methods of the present disclosure are utilized in a variety of practical applications. For example, in some implementations, a genotype for a tandem repeat region is evaluated, in combination with known genotype-drug response associations, to determine a risk of adverse drug reaction. As described above, responses to certain drugs have been observed to have a genetic basis, often in the genes that encode the enzymes involved in the metabolism of the drugs within the body. Thus, in some implementations, poor metabolism of certain therapeutic drugs can increase the risk of toxicity and/or potentially life-threatening side effects, whereas abnormally high metabolism of certain therapeutic drugs can lower the efficacy of treatment. In some implementations, the identified genotypes are useful for diagnosing a patient's sensitivity or resistance to a particular therapeutic agent. Such genotypes can further be used to guide treatment recommendations for individualized medicine, such as selecting therapeutic drugs, determining dosages, modifying existing treatments, and/or assigning treatment regimens. In some embodiments, the repeat polymorphism genotypes are used to predict an effect of a treatment with a cancer drug in a particular patient. In some embodiments, the repeat polymorphism genotypes are used to inform cancer diagnoses and/or prognoses for a patient.

In some embodiments, a patient is selected for enrollment in one or more clinical trials based on the determination of a particular tandem repeat genotype (e.g., in accordance with the patient's pharmacogenomic profile).

In some embodiments, the systems and methods of the present disclosure are used to provide any of the foregoing information on a clinical report (e.g., information regarding risk of drug reactions, therapeutic agent sensitivity or resistance, recommended treatments, and/or clinical trial enrollment).

In some embodiments, the systems and methods of the present disclosure are used to inform product labels for therapeutic drugs. In some embodiments, the systems and methods of the present disclosure are used to develop companion diagnostic tests, and/or serve as a component of a panel of tests to support an individualized medicine approach.

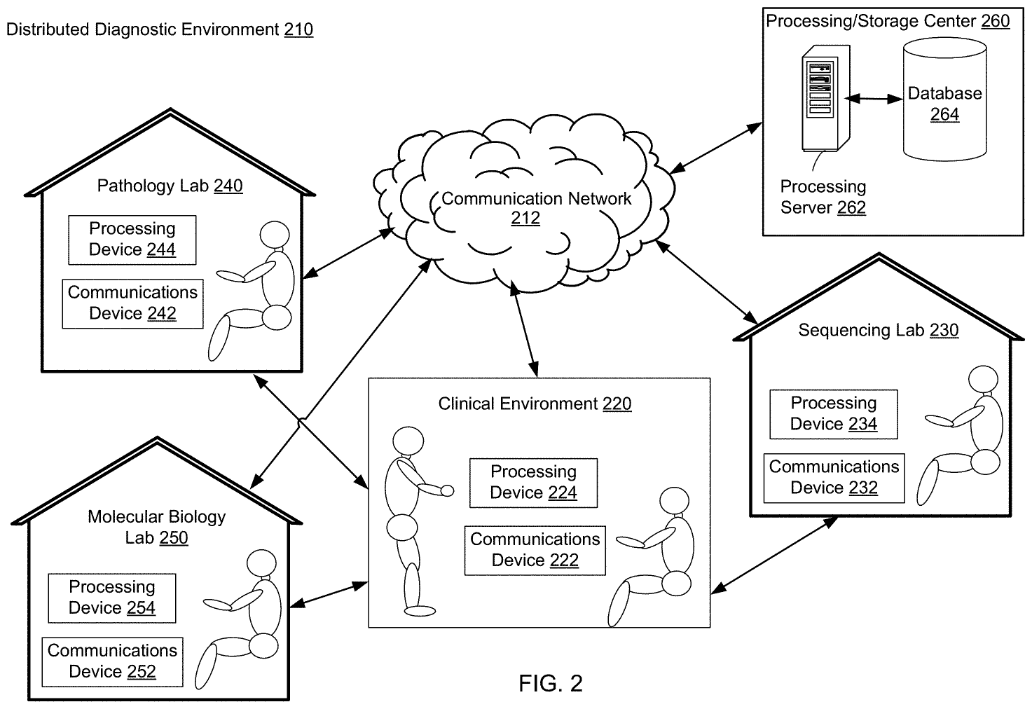

In some embodiments, the systems and methods disclosed herein are utilized in a research and/or a clinical setting (e.g., to elucidate heterogeneity of therapeutic responses within and among patients and/or to potentially guide precision oncology treatments). In some embodiments, the systems and methods disclosed herein are utilized in a distributed diagnostic environment, as illustrated in FIG. 2.

### B. Definitions

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” is intended to mean “when” or “upon” or “in response to determining” or “in response to detecting,” depending on the context. Similarly, the phrase “if it is determined” or “if [a stated condition or event] is detected” is intended to mean “upon determining” or “in response to determining” or “upon detecting [the stated condition or event]” or “in response to detecting [the stated condition or event],” depending on the context.

As disclosed herein, each numerical value is intended to be read once as modified by the term “about” (unless already expressly so modified), and then read again as not so modified unless otherwise indicated in context. Also, it will be understood that a physical range listed or described as being useful, suitable, or the like, is intended such that any and every value within the range, including the end points, is to be considered as having been stated. For example, “a range of from 1 to 10” is to be read as indicating each and every possible number along the continuum between about 1 and about 10. Thus, even if specific data points within the range, or even no data points within the range, are explicitly identified or refer to only a few specific data points, it is to be understood that any and all data points within the range are to be considered to have been specified, and that the present disclosure shows possession of knowledge of the entire range and all points within the range.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “allele” refers to a particular sequence of one or more nucleotides at a chromosomal locus. As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species. As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species.

As used herein, the terms “alignment” and “aligning” refer to the process of comparing a read to a reference sequence and thereby determining whether the reference sequence contains the read sequence. In some embodiments, an alignment process attempts to determine if a read can be mapped to a reference sequence, but does not always result in a read aligned to the reference sequence. In certain embodiments, if the reference sequence contains the read, the read is mapped to the reference sequence or to a particular location in the reference sequence. In some cases, alignment indicates whether or not a read is a member of a particular reference sequence (e.g., whether the read is present or absent in the reference sequence). In an example instance, the alignment of a read to the reference sequence for human chromosome 13 indicates whether the read sequence is present in the reference sequence for chromosome 13. In some embodiments, a tool that provides this information is referred to as a set membership tester. In some cases, an alignment additionally indicates a location in the reference sequence to which the read maps. For example, if the reference sequence is the whole human genome sequence, in some embodiments, an alignment indicates that a read is present on chromosome 13, and further indicates that the read is on a particular strand and/or site of chromosome 13.

As used herein, the term “aligned reads” refer to one or more sequences that are identified as a match in terms of the order of their nucleic acid molecules to a known reference sequence such as a reference genome. In some embodiments, an aligned read and its determined location on the reference sequence is referred to as a sequence tag. Typically, alignment is implemented by a computer algorithm, as it would be impossible to align reads in the human mind or using pen and paper in a reasonable time period in order to implement the methods disclosed herein. One example of an algorithm from aligning sequences is the Smith-Waterman algorithm. Another is the Efficient Local Alignment of Nucleotide Data (ELAND) computer program. Alternatively, in some implementations, a Bloom filter or similar set membership tester is employed to align reads to reference genomes. In some embodiments, the matching of a sequence read to a reference sequence during alignment results in a 100% sequence match or a less than 100% sequence match (e.g., a non-perfect match).

As used herein, the term “assay” refers to a technique for determining a property of a substance, e.g., a nucleic acid, a protein, a cell, a tissue, or an organ. Any assay known to a person having ordinary skill in the art is contemplated for use in detecting any of the properties of nucleic acids mentioned herein. In some embodiments, properties of a nucleic acid include a sequence, genomic identity, genotype, copy number, methylation state at one or more nucleotide positions, size of the nucleic acid, presence or absence of a mutation in the nucleic acid at one or more nucleotide positions, and/or pattern of fragmentation of a nucleic acid (e.g., the nucleotide position(s) at which a nucleic acid fragments). In some embodiments, an assay or method has a particular sensitivity and/or specificity, and its relative usefulness as a diagnostic tool is measured using ROC-AUC statistics.

As used herein, the term “based on,” when used in the context of obtaining a specific quantitative value, refers to using another quantity as input to calculate the specific quantitative value as an output.

As used herein, the term “biological fluid” refers to a liquid taken from a biological source and includes, for example, blood, serum, plasma, sputum, lavage fluid, cerebrospinal fluid, urine, semen, sweat, tears, saliva, and the like. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

As used herein, the term “cancer,” “cancerous tissue,” or “tumor” refers to an abnormal mass of tissue in which the growth of the mass surpasses and is not coordinated with the growth of normal tissue. Included in this definition are benign and malignant cancers as well as dormant tumors or micrometastases. In some embodiments, a cancer or tumor is defined as “benign” or “malignant” depending on the following characteristics: degree of cellular differentiation including morphology and functionality, rate of growth, local invasion, and/or metastasis. In some embodiments, a “benign” tumor is well differentiated, has characteristically slower growth than a malignant tumor, and/or remains localized to the site of origin. In addition, in some cases, a benign tumor does not have the capacity to infiltrate, invade or metastasize to distant sites. In some embodiments, a “malignant” tumor is poorly differentiated (anaplasia) and/or has characteristically rapid growth accompanied by progressive infiltration, invasion, and destruction of the surrounding tissue. Furthermore, in some embodiments, a malignant tumor has the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

As used herein, the term “chromosome” refers to the heredity-bearing gene carrier of a living cell, which is derived from chromatin strands comprising DNA and protein components (especially histones). The conventional internationally recognized individual human genome chromosome numbering system is employed herein.

As used herein, the term “classification” can refer to any number(s) or other characters(s) that are associated with a particular property of a sample. For example, in some embodiments, the term “classification” refers to a genotype of a subject at a genomic locus comprising a tandem repeat, a likelihood that a subject comprises a respective candidate allele, a likelihood that a subject comprises a pair of candidate alleles, a risk of adverse drug reaction for a subject, and the like. In some embodiments, the classification is binary (e.g., positive or negative) or has two or more levels of classification (e.g., a scale from 1 to 10 or 0 to 1). In some embodiments, the terms “cutoff” and “threshold” refer to predetermined numbers used in an operation. For example, in some implementations, a cutoff size refers to a size above which fragments are excluded. In some embodiments, a threshold value is a value above or below which a particular classification applies. Either of these terms are suitable for use in either of these contexts.

As used herein, the term “clinically relevant” refers to anything that is known or is suspected to be associated or implicated with a genetic or disease condition. For instance, in some embodiments, a “clinically relevant” sequence or genotype is a nucleic acid sequence that is suspected to be associated or implicated with a genetic or disease condition. In some implementations, determining the absence or presence of a clinically relevant sequence or genotype is useful for determining a diagnosis or confirming a diagnosis of a medical condition, or providing a prognosis for the development of a disease.

As used herein, the term “derived,” when used in the context of a nucleic acid or a mixture of nucleic acids, refers to the means whereby the nucleic acid(s) are obtained from the source from which they originate. For example, in one embodiment, a mixture of nucleic acids that is derived from two different genomes means that the nucleic acids, e.g., cfDNA, were naturally released by cells through naturally occurring processes such as necrosis or apoptosis. In another embodiment, a mixture of nucleic acids that is derived from two different genomes means that the nucleic acids were extracted from two different types of cells from a subject.

As used herein, the term “locus” or “site” refers to a position within a genome, e.g., on a particular chromosome and/or having a particular orientation. In some embodiments, a locus refers to a residue, a sequence tag, or a segment's position on a reference sequence. In some embodiments, a locus refers to a single nucleotide position within a genome, e.g., on a particular chromosome. In some embodiments, a locus refers to a small group of nucleotide positions within a genome, e.g., as defined by a mutation (e.g., substitution, insertion, or deletion) of consecutive nucleotides within a cancer genome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome.

As used herein, the term “mapping” refers to assigning a read sequence to a larger sequence, e.g., a reference genome. In some embodiments, mapping is performed by alignment.

As used herein, the term “Next Generation Sequencing (NGS)” refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

As used herein, the term “parameter” herein refers to a numerical value that characterizes a physical property. Frequently, a parameter numerically characterizes a quantitative data set and/or a numerical relationship between quantitative data sets. For example, a ratio (or function of a ratio) between the number of sequence tags mapped to a chromosome and the length of the chromosome to which the tags are mapped, is a parameter.

As used interchangeably herein, the terms “polynucleotide,” “nucleic acid” and “nucleic acid molecules” refer to a covalently linked sequence of nucleotides (e.g., ribonucleotides for RNA and deoxyribonucleotides for DNA) in which the 3′ position of the pentose of one nucleotide is joined by a phosphodiester group to the 5′ position of the pentose of the next. In some embodiments, nucleotides include sequences of any form of nucleic acid, including, but not limited to RNA and DNA molecules such as cell-free DNA (cfDNA) molecules. The term “polynucleotide” includes, without limitation, single- and double-stranded polynucleotides.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. A “genome” refers to the complete genetic information of an organism or virus, expressed in nucleic acid sequences.

In various embodiments, the reference sequence is significantly larger than the reads that are aligned to it. For example, in some embodiments, the reference sequence is at least about 100 times larger, at least about 1000 times larger, at least about 10,000 times larger, at least about 10⁵ times larger, at least about 10⁶ times larger, or at least about 10⁷ times larger.

In one example, the reference sequence is that of a full length human genome. In some embodiments, such sequences are referred to as genomic reference sequences. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). In another example, the reference sequence is limited to a specific human chromosome such as chromosome 13. In some embodiments, a reference Y chromosome is the Y chromosome sequence from human genome version hg19. In some embodiments, such sequences are referred to as chromosome reference sequences. Other examples of reference sequences include genomes of other species, as well as chromosomes, sub-chromosomal regions (such as strands), etc., of any species.

In some embodiments, a reference sequence for alignment has a sequence length from about 1 to about 100 times the length of a read. In such embodiments, the alignment and sequencing are considered a targeted alignment or sequencing, instead of a whole genome alignment or sequencing. In these embodiments, the reference sequence typically includes a gene and/or a repeat sequence of interest.

In various embodiments, the reference sequence is a consensus sequence or other combination derived from multiple individuals. However, in certain applications, the reference sequence may be taken from a particular individual.

As used herein, the term “repeat sequence” refers to a longer nucleic acid sequence including repetitive occurrences of a shorter sequence. The shorter sequence is referred to as a “repeat unit” herein. The repetitive occurrences of the repeat unit are referred to as “counts,” “repeats,” or “copies” of the repeat unit. In many contexts, a repeat sequence is associated with a gene encoding a protein. In other situations, a repeat sequence is in a non-coding region. In some embodiments, the repeat units occur in the repeat sequence with or without breaks between the repeat units. For instance, in normal samples, the FMR1 gene tends to include an AGG break in the CGG repeats, e.g., (CGG)10+(AGG)+(CGG)9. The term “tandem repeat,” as used herein, refers to a repeat sequence where the repeat units are contiguous. Repeat sequences lacking breaks, as well as long repeat sequences having few breaks, are prone to repeat expansion of the associated gene, which in some cases leads to genetic diseases as the repeats expand above a particular number. In various embodiments of the disclosure, the number of repeats is counted as in-frame repeats regardless of breaks. Methods for estimating in-frame repeat polymorphisms are further described hereinafter.

In various embodiments, the repeat units include 2 to 100 nucleotides. Many repeat units widely studied are trinucleotide or hexanucleotide units. Some other repeat units that have been well studied and are applicable to the embodiments disclosed herein include but are not limited to units of 4, 5, 6, 8, 12, 33, or 42 nucleotides. See, e.g., Richards, Human Molecular Genetics, 10: 20, 2187-2194 (2001). Applications of the disclosure are not limited to the specific number of nucleotide bases described above, so long as they are relatively short compared to the repeat sequence having multiple repeats or copies of the repeat units. For example, in some instances, a repeat unit includes at least 2, 3, 6, 8, 10, 15, 20, 30, 40, or 50 nucleotides. Alternatively or additionally, in some embodiments, a repeat unit includes at most about 100, 90, 80, 70, 60, 50, 40, 30, 20, 10, 6 or 3 nucleotides.

In some embodiments, a repeat sequence forms a polymorphism through evolution, development, or mutagenic conditions, creating more or less copies of the same repeat unit. This process is also referred to as “dynamic mutation” due to the unstable nature of the repeat unit number. Some repeat polymorphisms have been shown to be associated with genetic disorders and pathological symptoms. Other repeat polymorphisms are not well understood or studied. In some embodiments, the disclosed methods herein are used to identify both previously known and new, unknown repeat polymorphisms. In some embodiments, a repeat sequence polymorphism is longer than about 5 base pairs (bp), about 10 bp, about 20 bp, about 50 bp, about 100 bp, about 200 bp, or about 500 bp. In some embodiments, a repeat sequence polymorphism is longer than about 1000 bp, 2000 bp, 3000 bp, 4000 bp, 5000 bp, or more. In some embodiments, a repeat sequence polymorphism is no longer than about 10,000 bp, about 5000 bp, about 2000 bp, about 1000 bp, about 500 bp, about 100 bp, about 50 bp, about 20 bp, about 10 bp, or less.

As used herein, the term “repeat sequence genotype” refers to the nucleic acid sequence of the area of the genome that includes the sequence of the repeat units and any sequence breaks comprised therein.

As used herein, the term “report” denotes a form of clinical or research decision-making support, including clinically or research-relevant genotype information concerning repeat polymorphism that can be used by a clinician or researcher. In some embodiments, information includes, but is not limited to, the accurate genotype of the repeat polymorphisms present in the sample; identification of those genotypes that are associated with a reduced ability respond to a therapy or drug; identification of genotypes that are associated with an increased likelihood of an adverse side effect with a therapy or drug; identification of genotypes known to affect disease course or prognosis; and/or genotypes that can help with diagnosis (see, Beaubier et al., Nat. Biotechnol., 37(11):1351-60 (2019)).

As used herein, the term “sample,” “biological sample,” or “test sample” herein refers to a sample, typically derived from a biological fluid, cell, tissue, organ, or organism, that includes a nucleic acid or a mixture of nucleic acids having at least one nucleic acid sequence that is to be assayed for determining a repeat sequence genotype. In certain embodiments, the sample has at least one nucleic acid sequence comprising a repeat sequence that is suspected of having undergone variation. Such samples include, but are not limited to sputum/oral fluid, amniotic fluid, blood, blood fractions, fine needle biopsy samples, urine, peritoneal fluid, pleural fluid, and the like. Although the sample is often taken from a human subject (e.g., a patient), in some embodiments, repeat sequence genotypes are determined using samples from any mammal, including, but not limited to, dogs, cats, horses, goats, sheep, cattle, and/or pigs. In some embodiments, the sample is used directly as obtained from the biological source or following a pretreatment to modify the character of the sample. For example, in some implementations, such pretreatment includes preparing plasma from blood, diluting viscous fluids, and so forth. In some embodiments, methods of pretreatment also involve, but are not limited to, filtration, precipitation, dilution, distillation, mixing, centrifugation, freezing, lyophilization, concentration, amplification, nucleic acid fragmentation, inactivation of interfering components, the addition of reagents, and/or lysing. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

In some embodiments, the term “control sample” refers to a negative or positive control sample. A “negative control sample” or “unaffected sample” refers to a sample including nucleic acids that is known or expected to have a repeat sequence having a number of repeats within a range that is not pathogenic. A “positive control sample” or “affected sample” is known or expected to have a repeat sequence having a number of repeats within a range that is pathogenic. Repeats of the repeat sequence in a negative control sample typically have not been expanded beyond a normal range, whereas repeats of a repeat sequence in a positive control sample typically have been expanded beyond a normal range. As such, in some embodiments, the nucleic acids in a test sample can be compared to one or more control samples.

In some embodiments, the term “patient sample” refers to a biological sample obtained from a patient, e.g., a recipient of medical attention, care or treatment. In some embodiments, the patient sample is any of the samples described herein. In certain embodiments, the patient sample is obtained by non-invasive procedures, e.g., a peripheral blood sample or a stool sample. The methods described herein need not be limited to humans. Thus, various veterinary applications are contemplated in which case the patient sample may be a sample from a non-human mammal (e.g., a feline, a porcine, an equine, a bovine, and the like).

In some embodiments, the term “normal sample” refers to a sample from a subject that does not have a particular condition, or is otherwise healthy. In an example, a method as disclosed herein can be performed on a first subject having a tumor, where the normal sample is a sample taken from a healthy tissue of the first subject, or from a second subject that does not have a tumor.

As used herein, the terms “sequence of interest” or “genotype of interest” refer to a nucleic acid sequence that is associated with a difference in sequence representation in healthy versus diseased individuals. In some embodiments, a sequence of interest is a repeat sequence on a chromosome that is expanded or contracted in a disease or genetic condition. In some embodiments, a sequence of interest is a portion of a chromosome, a gene, a coding sequence or a non-coding sequence. In some embodiments, the term “corresponding to” when used in the context of a nucleic acid sequence, e.g., a gene or a chromosome, refers to a nucleic acid sequence that is present in the genome of different subjects, and which does not necessarily have the same sequence in all genomes, but serves to provide the identity rather than the genetic information of a sequence of interest, e.g., a gene or chromosome.

As used herein, the terms “sequencing,” “sequence determination,” and the like refers generally to any and all biochemical processes used to determine the order of biological macromolecules such as nucleic acids or proteins. For example, in some embodiments, sequencing data includes all or a portion of the nucleotide bases in a nucleic acid molecule such as an mRNA transcript or a genomic locus.

As used herein, the term “sequence read” or “read” refers to a sequence read from a portion of a nucleic acid sample. Typically, though not necessarily, a read represents a short sequence of contiguous base pairs in the sample. In some embodiments, a read is represented symbolically by the base pair sequence (in ATCG) of the sample portion. In some cases, a read is stored in a memory device and processed as appropriate to determine whether it matches a reference sequence or meets other criteria. In some instances, a read is obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and mapped to a chromosome or genomic region or gene.

In some embodiments, sequence reads are produced by any sequencing process described herein or known in the art. In some cases, reads are generated from one end of nucleic acid fragments (“single-end reads”) or from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. In some embodiments, sequence reads are obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “subject” refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

As used herein, the term “mutation” or “variant” refers to a detectable change in the genetic material of one or more cells. In a particular example, one or more mutations can be found in, and can identify, cancer cells (e.g., driver and passenger mutations). In some embodiments, a mutation is transmitted from a parent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that is added, deleted, substituted for, inverted, or transposed to a new position in a nucleic acid. In some instances, a mutation is a spontaneous mutation or an experimentally induced mutation. A mutation in the sequence of a particular tissue is an example of a “tissue-specific allele.” For example, in some implementations, a tumor has a mutation that results in an allele at a locus that does not occur in normal cells. Another example of a “tissue-specific allele” is a fetal-specific allele that occurs in the fetal tissue, but not the maternal tissue.

As used herein, the term “single nucleotide variant” or “SNV” refers to a substitution of one nucleotide to a different nucleotide at a position (e.g., site) of a nucleotide sequence, e.g., a sequence read from an individual. In an example embodiment, a substitution from a first nucleobase X to a second nucleobase Y is denoted as “X>Y.” For instance, in such an embodiment, a cytosine to thymine SNV is denoted as “C>T.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### C. Example System Embodiments

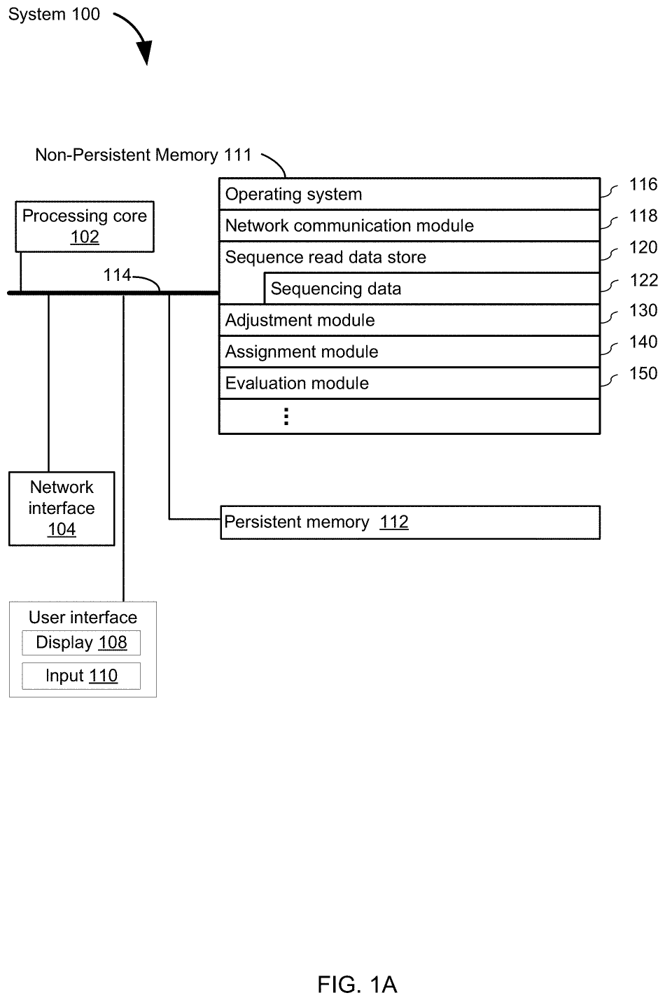

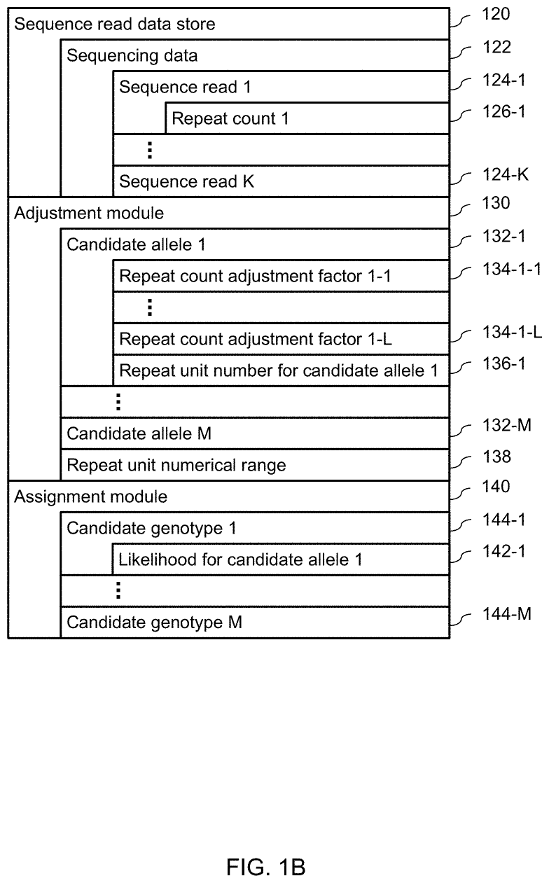

Now that an overview of some aspects of the present disclosure and some definitions used in the present disclosure have been provided, details of an exemplary system are now described in conjunction with FIGS. 1A-B. FIG. 1 is a block diagram illustrating a system 100 in accordance with some implementations. The device 100 in some implementations includes one or more processing units CPU(s) 102 (also referred to as processors), one or more network interfaces 104, a user interface 106, optionally comprising a display 108 and input 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network;
a sequence read data store 120 for storing data sets containing sequencing data 122 for at least a first set of sequence reads;
an adjustment module 130 for storing data sets containing adjustment factors;
an assignment module 140 for assigning likelihoods of candidate allele identities; and
an evaluation module 150 for selecting one or more candidate alleles.

Referring to FIG. 1B, optionally, each sequence read 124 (e.g., 124-1, . . . 124-K) in the at least the first set of sequence reads maps to a genomic locus comprising a tandem repeat, where the tandem repeat consists of a plurality of contiguous nucleotide repeat units. In some embodiments, each respective sequence read 124 encompasses the tandem repeat. Optionally, the sequence read data store 120 further comprises, for each sequence read 124 in the at least the first set of sequence reads, a corresponding repeat count 126 (e.g., 126-1) for the number of repeat units in the plurality of contiguous nucleotide repeat units.

In some embodiments, the adjustment module 130 further comprises, for each respective candidate allele 132 (e.g., 132-1, . . . 132-M) in a plurality of candidate alleles, a corresponding set of repeat count adjustment factors 134 (e.g., 134-1-1, . . . 134-1-L) in a plurality of sets of repeat count adjustment factors. In some such embodiments, each respective candidate allele 132 in the plurality of candidate alleles has a different corresponding number of repeat units 136 (e.g., 136-1) for the plurality of contiguous nucleotide repeat units, and each respective set of repeat count adjustment factors includes a corresponding repeat count adjustment factor for each respective number of repeat units in a numerical range 138 of repeat units for the plurality of contiguous nucleotide repeat units.

In some embodiments, the assignment module 140 further comprises, for each respective candidate genotype 144 in a plurality of candidate genotypes for the genomic locus (e.g., 144-1, . . . 144-M), a corresponding first likelihood 142 (e.g., 142-1) for the respective candidate genotype. In some embodiments, the corresponding first likelihood is based upon, for each respective candidate allele 132 corresponding to the respective candidate genotype, (i) a proportion of sequence reads 124 in the plurality of sequence reads that have the repeat count 126 of the number of repeat units in the plurality of contiguous repeat units corresponding to the respective candidate allele and (ii) a repeat count adjustment factor 134 matching the number of repeat units in the plurality of contiguous repeat units corresponding to the respective candidate allele from the set of repeat count adjustment factors for the respective candidate allele.

In some embodiments, the evaluation module 150 is used to evaluate and/or select the respective candidate genotype 144 in the plurality of candidate genotypes having the highest corresponding likelihood 142, thereby determining the genotype of the subject for the genomic locus comprising the tandem repeat.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, data sets, or modules, and thus, in some embodiments, various subsets of these modules and data are combined or otherwise re-arranged in various implementations.

In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of system 100, that is addressable by system 100 so that system 100 retrieves all or a portion of such data when needed.

Although FIGS. 1A-B depict a “system 100,” the figure is intended more as a functional description of the various features which may be present in one or more computer systems than as a structural schematic of the implementations described herein. In practice, and as recognized by those of ordinary skill in the art, items shown separately could be combined and some items could be separated. Moreover, although FIGS. 1A-B depict certain data and modules in non-persistent memory 111, some or all of these data and modules may be in persistent memory 112.

For instance, as depicted in FIG. 2, in some embodiments, the methods described herein are performed across a distributed diagnostic environment 210, e.g., connected via communication network 212. In some embodiments, one or more biological samples, e.g., one or more tumor biopsy or normal samples, are collected from a subject in clinical environment 220, e.g., a doctor's office, hospital, or medical clinic. In some embodiments, the one or more samples, or a portion thereof, are processed within the clinical environment using a processing device 224, e.g., a nucleic acid sequencer for obtaining sequencing data, a microscope for obtaining pathology data, a mass spectrometer for obtaining proteomic data, etc. In some embodiments, the one or more biological samples, or a portion thereof, are sent to one or more external environments, e.g., sequencing lab 230, pathology lab 240, and molecular biology lab 250, each of which includes a processing device 234, 244, and 254, respectively, to generate biological data about the subject. Each environment includes a communications device 222, 232, 242, and 252, respectively, for communicating biological data about the subject to a processing server 262 and/or database 264, optionally located in yet another environment, e.g., processing/storage center 260. Thus, in some embodiments, different portions of the systems and methods described herein are fulfilled by different processing devices located in different physical environments.

### D. Example Embodiments for Genotyping Repeat Sequences

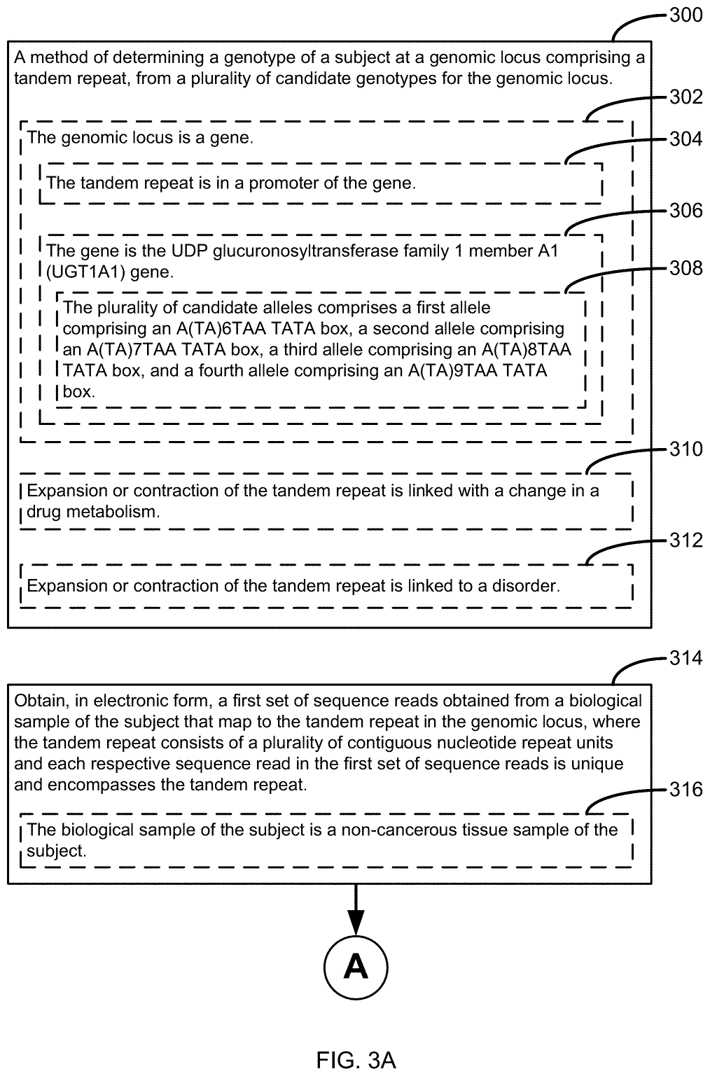

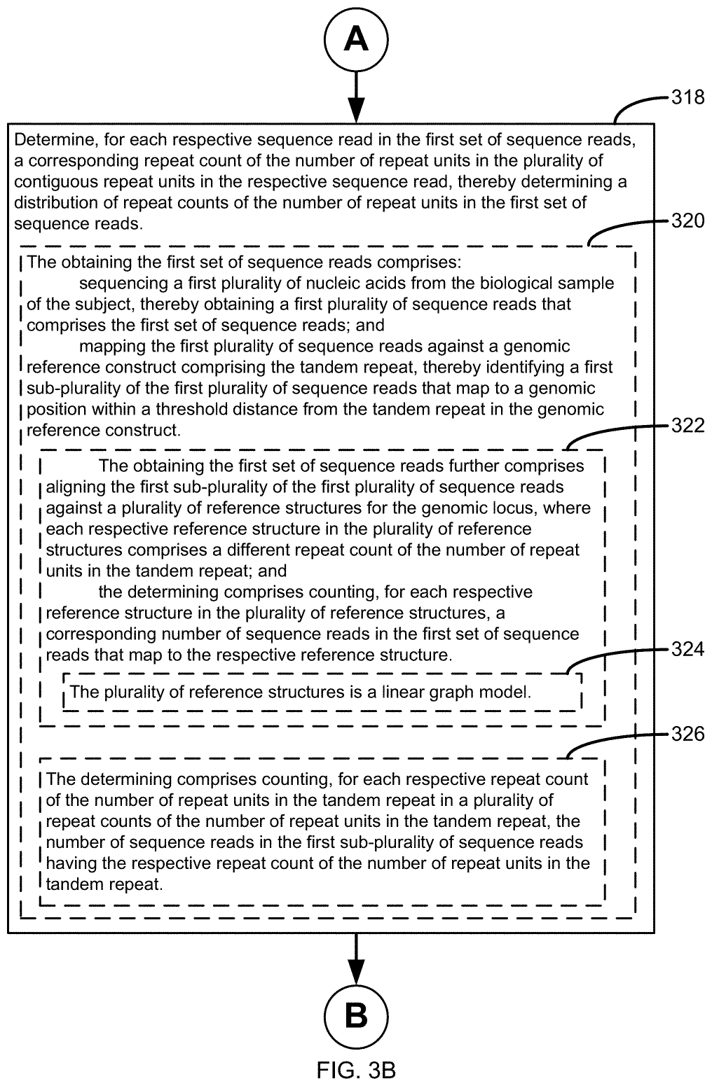

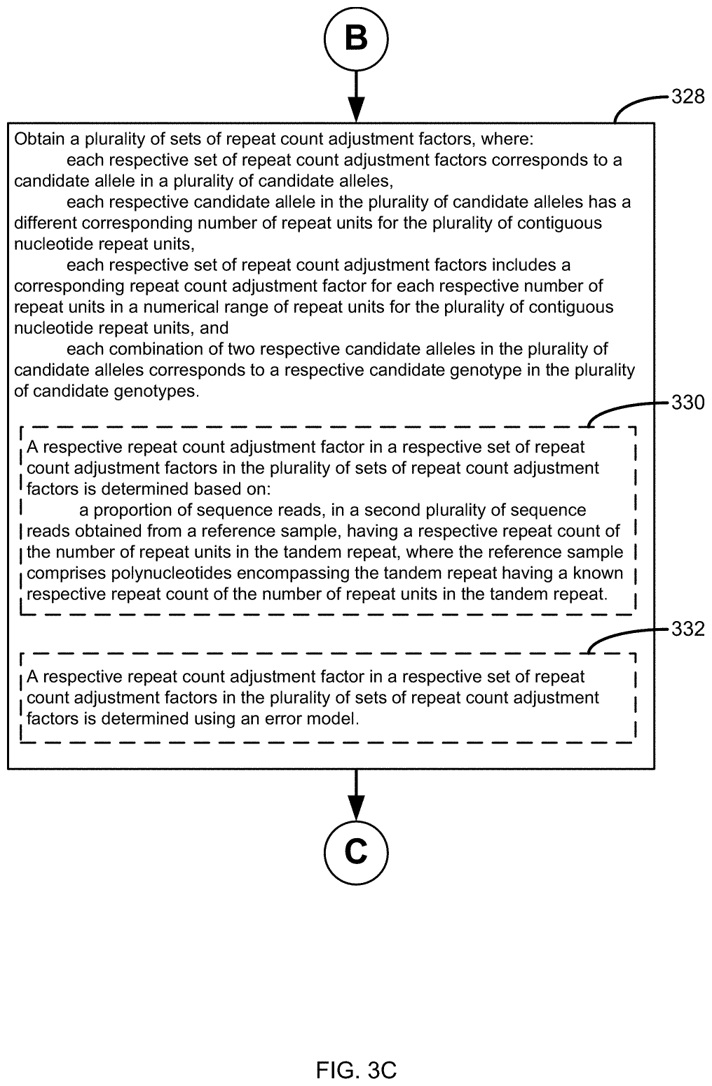

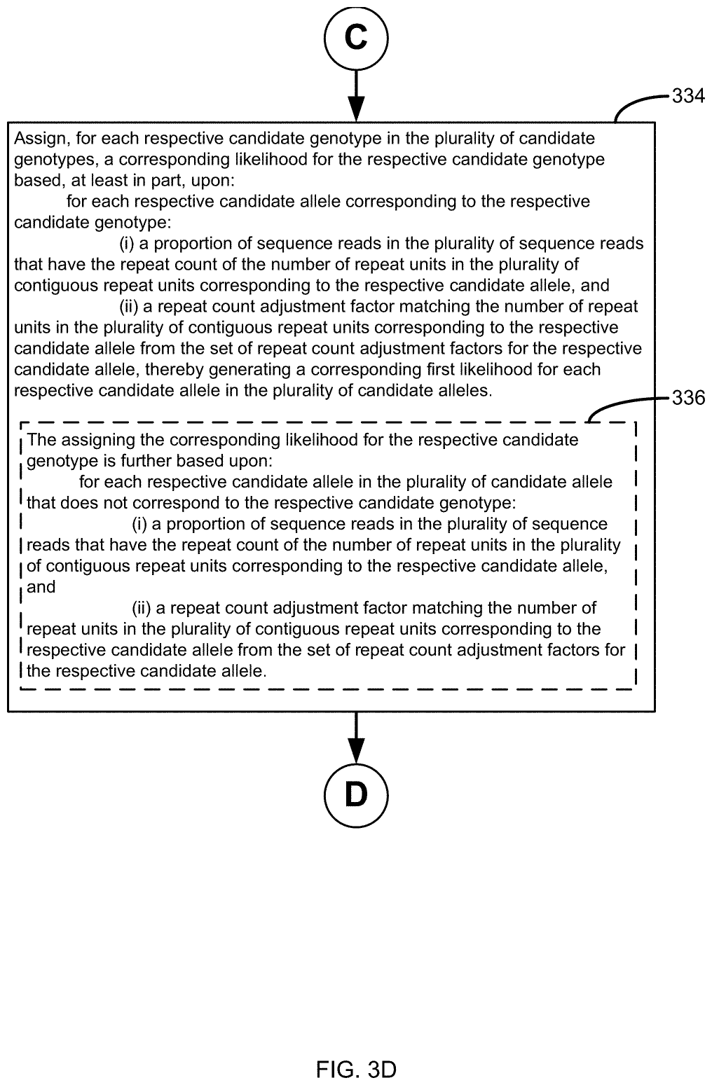

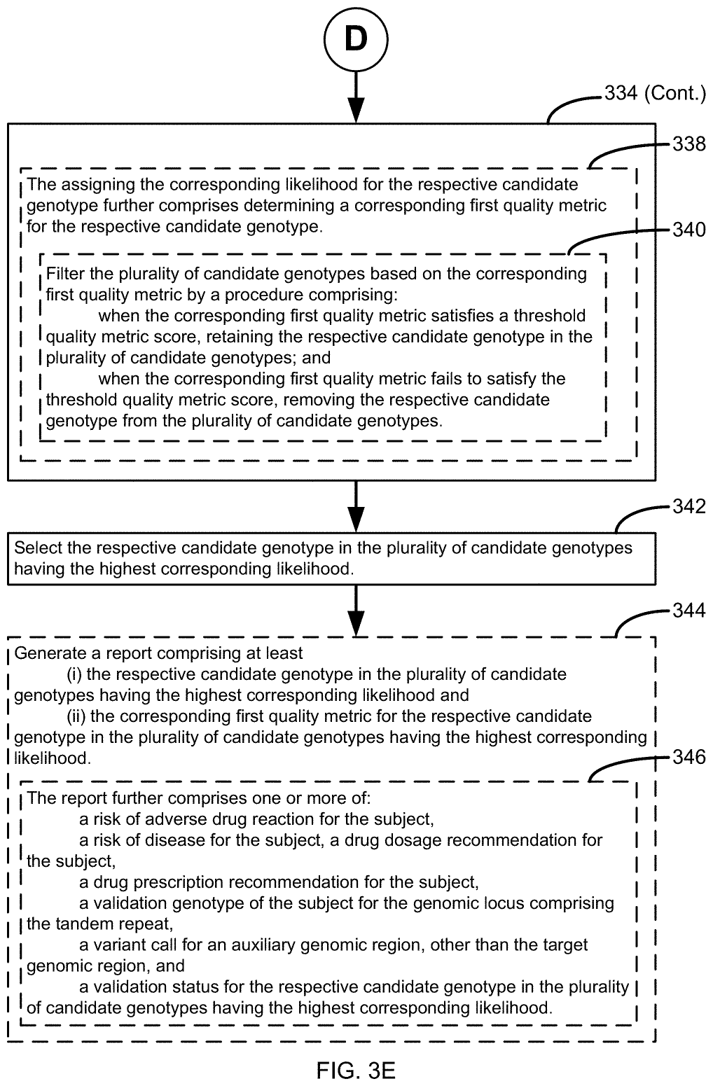

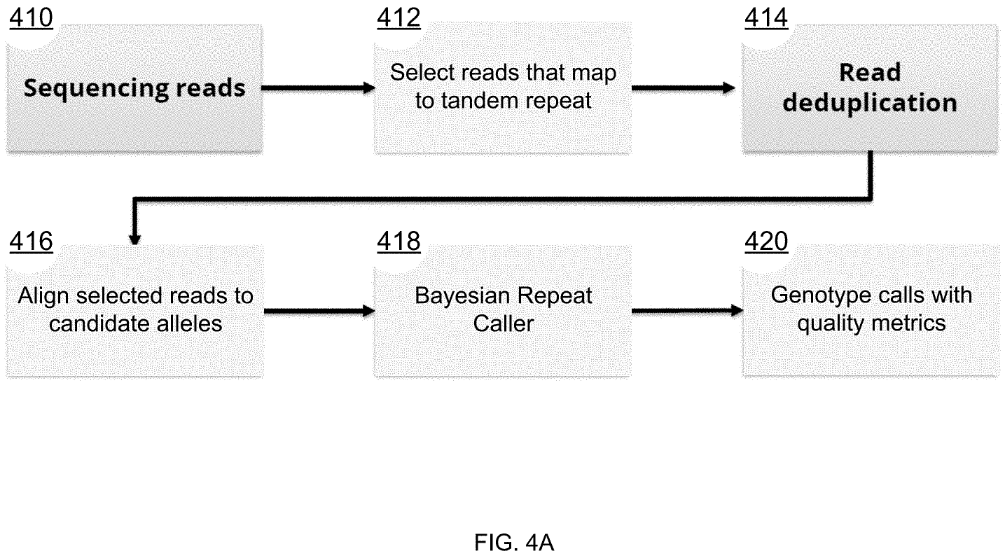

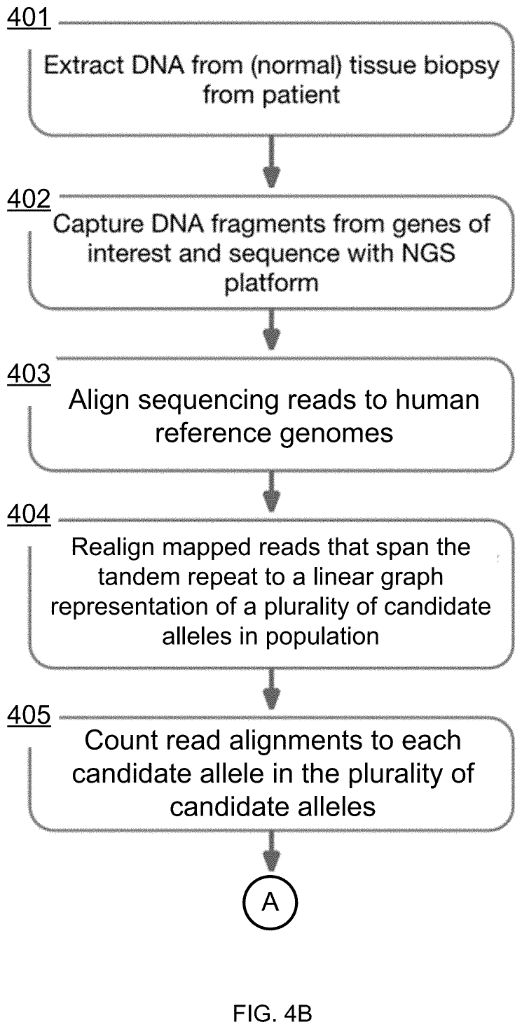

While a system in accordance with the present disclosure has been disclosed with reference to FIGS. 1 and 2, methods in accordance with the present disclosure are now detailed with reference to FIGS. 3A-E and FIGS. 4A-C.

In some aspects, the present disclosure provides at least systems and methods for accurate genotyping of dinucleotide and other short tandem repeat alleles from NGS short read alignments, eliminating the shortcomings and artifacts mentioned above, and thus enabling clinical testing of clinically relevant repeat variants, such as but not limited to the UGT1A1 promoter repeat, from targeted capture NGS in conjunction with other genetic variants. In one embodiment, the NGS panel is a targeted panel designed to analyze the sequences of a set of genes relevant for precision medicine in the field of oncology and the panel is applied to at least one cancer specimen collected from a patient and at least one non-cancer specimen collected from the same patient. In some embodiments, a whole transcriptome RNA-seq panel is applied to at least the cancer specimen in conjunction with the NGS panel. In various embodiments, cell-free nucleic acids and/or cell-associated nucleic acids of either or both specimens are analyzed by one or more sequencing panels (e.g., an NGS panel and/or an RNA-seq panel). In some embodiments, the analysis of the one or more specimens is used to detect one or more of: 1) somatic genetic variants in the patient's cancer specimen, especially variants that are absent from the patient's non-cancer specimen, 2) an RNA expression level for each gene in the transcriptome for the cancer and/or non-cancer specimen, and 3) germline genetic variants in the non-cancer specimen, especially in genes involved in drug metabolism or pharmacokinetics (for example, UGT1A1, DPYD, and the like). An example of NGS analysis of genes involved in drug metabolism or pharmacokinetics, is disclosed, for example, in U.S. Pat. No. 10,978,196, titled “Data-based mental disorder research and treatment systems and methods,” and published Apr. 13, 2021, which is incorporated herein by reference and in its entirety for all purposes.

1. Example Repeat Sequences

In some embodiments, the presently disclosed systems and methods are used for the determination of a genotype for the UGT1A1 gene. In particular, in some embodiments, the presently disclosed systems and methods is used to determine a repeat sequence polymorphism of the UGT1A1 gene. This gene encodes the enzyme responsible for the glucuronidation of SN-38, the active metabolite of irinotecan (IRI), thus facilitating clinical approaches to the use of this drug that take the repeat sequence polymorphism into account (see, e.g., Nelson et al., Cancers, 13: 1566 (2021)). Wild-type UGT1A1 contains six TA repeats [A(TA)₆TAA] in its promoter region (also known as the *1 allele). Polymorphic UGT1A1 alleles with a higher number of TA repeats, such as UGT1A1*28/(TA)₇ and *37/(TA)₈ alleles, cause decreased enzyme activity and are associated with severe toxicity in patients receiving IRI-based chemotherapy, where, in some such cases, dose reductions are recommended. Additionally, a polymorphic UGT1A1 allele with a lower number of TA repeats known as UGT1A1*36/(TA)₅ has enzyme activity that is greater than or equal to normal limits (e.g., wild-type activity).

Other variations in drug side effects have also been associated with UGT1A1 polymorphism, such as with the administration of belinostat, pazopanib, and nilotinib. Non-limiting examples of various clinically relevant alleles of the UGT1A1 promoter are provided in Table 1. In various embodiments, the systems and methods herein are used to detect UGT1A1 promoter alleles having repeats of between approximately 1 and 20. In some embodiments, the present methods and systems are utilized to develop an exemplary system to detect repeat sequence polymorphisms of the UGT1A1 gene and therefore associate possible clinical decisions with the findings, even if a novel detected allele has not been previously associated with a phenotype and/or clinical effect. In some implementations, these novel associations are based on analysis of clinical trials, biochemical assays, in vitro assays, in vivo assays, and/or clinical records associated with the patients having the allele.

TABLE 1
Common UGT1A1 Promoter Alleles

| Allele Name | Repeat Genotype | Enzyme Expression Impact |
|---|---|---|
| UGT1A1*1 | (TA)₆TAA | None; wildtype |
| UGT1A1*36 | (TA)₅TAA | Increased |
| UGT1A1*28 | (TA)₇TAA | Reduced |
| UGT1A1*37 | (TA)₈ TAA | Reduced |

The metabolism of many drugs are known to be impacted by the UGT-associated genes. Non-limiting examples include abiraterone, acalabrutinib, asciminib, anastrozole, axitinib, belinostat, bendamustin, bexarotene, bicalutamide, binimetinib, bleomycin, camptothecin, cerdulatinib, chlorambucil, cobimetinib, cytarabine, dasatinib, daunorubicin, doxorubicin, duvelisib, enasidenib, encorafenib, epirubicin, erlotinib, etoposide, exemestane, fenretinide, flavopiridol, fludarabine, 5-fluorouracil, fluoxymesterone, flumatinib, fostamatinib, fulvestrant, glasdegib, ibrutinib, idelalisib, imatinib, irinotecan, isotretinoin, larotrectinib, letrozole, lorlatinib, medroxyprogesterone, megestrol, methotrexate, mitoxantrone, nintedanib, niraparib, olaparib, palbociclib, panobinostat, pomalidomide, raloxifene, regorafenib, ribavirin, ruxolitinib, selinexor, sorafenib, sunitinib, talazoparib, tamoxifen, thalidomide, tipifarnib, topotecan, toremifene, trabectedin, trametinib, tretinoin, vandetanib, venetoclax, vismodegib, and vorinostat. The possibility of side effects of one or more of these drugs, and any recommended increases or decreases in dosage in view of such side effects, are in some embodiments associated with specific genotypes of repeat polymorphisms, as detected and determined with the present methods and systems.

A further embodiment of the present methods and systems involves detecting and determining accurate genotypes of repeat polymorphisms that exist in microsatellite and minisatellite variants. A number of genetic disorders are caused by unstable repeat genotypes such as Fragile X syndrome, amyotrophic lateral sclerosis (ALS), Huntington's disease, Friedreich's ataxia, spinocerebellar ataxia, spino-bulbar muscular atrophy, myotonic dystrophy, Machado-Joseph disease, or dentatorubral pallidoluysian atrophy. Table 2 exemplifies non-limiting pathogenic repeat expansions that are different from repeat sequences in normal samples. The columns show genes associated with the repeat sequences, the nucleic acid sequences of the repeat units, the numbers of repeats of the repeat units for normal and pathogenic sequences, and the diseases associated with the repeat polymorphisms.

TABLE 2
Examples of Clinically Relevant Repeat Polymorphisms

| Gene | Repeat | Normal | Pathogenic | Disease |
|---|---|---|---|---|
| FMR1 | CGG | 6-60 | 200-900 | Fragile X |
| AR | CAG | 9-36 | 38-62 | Spino-bulbar |
| | | | | muscular atrophy |
| GHTT | CAG | 11-34 | 40-121 | Huntington's Disease |
| FXN | GAA | 6-32 | 200-1700 | Fredreich's ataxia |
| ATXN1 | CAG | 6-39 | 40-82 | Spinocerebellar ataxia |
| ATXN10 | ATTCT | 10-20 | 500-4500 | Spinocerebellar ataxia |
| ATXN2 | CAG | 15-24 | 32-200 | Spinocerebellar ataxia |
| ATXN3 | CAG | 13-36 | 61-84 | Spinocerebellar ataxia |
| ATXN7 | CAG | 4-35 | 37-306 | Spinocerebellar ataxia |
| C9ofr72 | GGGGCC | <30 | 100s | ALS |

Various general properties of repeat-related diseases have been identified in multiple studies. Dynamic mutation involving repeats is usually manifested as an increase in repeat number, with mutation rate being related to the number of repeats. Rare events such as loss of repeat interruption can lead to alleles having an increased likelihood of expanding, with such events being known as founder events. In some instances, relationships can exist between the number of repeats in the repeat sequence and the severity and/or onset of the disease caused by repeat expansion. It is contemplated that the present methods and systems have utility in accurate detection of these and other clinically relevant genotypes of repeat polymorphisms.

2. Example Workflows

FIGS. 4A-C present representative workflows of embodiments of the present methods and systems. A brief introductory overview of the workflows illustrated in FIGS. 4A-C follows.

In a first illustrative workflow, depicted in FIG. 4A, a first plurality of sequence reads 410 are obtained. The sequence reads are mapped to a reference sequence to generate read alignments 412. In some embodiments, sequence reads that map to a genomic locus comprising a tandem repeat are selected, thereby obtaining a first set of sequence reads. In some embodiments, the reference sequence contains a genomic locus comprising a repeat sequence (e.g., a tandem repeat), where the repeat sequence consists of a plurality of contiguous nucleotide repeat units. In some embodiments, each respective sequence read in the first set of sequence reads encompasses the repeat sequence. Optionally, the aligned reads are preprocessed, such as by removing duplicate reads 414 (e.g., read deduplication).

In some embodiments, for each respective sequence read in the first set of sequence reads, a corresponding repeat count of the number of repeat units in the repeat sequence is determined. For instance, in some embodiments, the selected reads (e.g., sequence reads that map to the tandem repeat in the genomic locus) are realigned 416 to linear graph models that collectively represent a plurality of candidate alleles having different corresponding number of repeat units for the plurality of contiguous nucleotide repeat units in the tandem repeat. In some embodiments, the selected reads are realigned to the possible repeat lengths expected in a reference population for the set of sequence reads (e.g., a population of reference genomes and/or subjects), where each respective linear graph model includes a corresponding repeat count of the number of repeat units in the repeat sequence, thus obtaining a respective repeat count for each respective sequence read.

In some embodiments, the illustrative workflow further comprises using a variant caller with the set of repeat counts corresponding to the first set of sequence reads. In some such embodiments, the variant caller is based on Bayes' theorem (e.g., a Bayesian repeat caller) 418. In some embodiments, the variant caller identifies a genotype call and/or a quality metric for the genomic locus containing the repeat sequence 420. Variant callers for determining genotypes for repeat sequences are described in greater detail below (see, e.g., the sections entitled “Assigning likelihood of candidate genotypes” and “Adjustment factors”).

A second illustrative workflow is depicted in FIGS. 4B-C. In some embodiments, DNA or another nucleic acid of interest is extracted from a sample of a subject 401 (e.g., biopsy tissue, saliva, blood, or another biological sample). Suitable methods for nucleic acid extraction are described more fully below (see, e.g., the section entitled “Nucleic acid extraction from biological samples”). In an embodiment of the present methods and systems, the sample is a normal tissue or other biological specimen that reflects germline, inherited variants.

In some embodiments, one or more regions of interest, such as a genomic locus that spans a repeat sequence of interest (e.g., that comprises a tandem repeat), are captured from input DNA fragments by any suitable method and are prepared into an NGS library 402. Various methods are contemplated for use in preparing sequencing libraries, as described more fully below (see, e.g., the section entitled “Sequencing library preparation”). In some implementations, library preparation is followed by nucleic acid sequencing 402. In some embodiments, the sequencing is NGS performed on a high-throughput short read sequencing instrument to produce coverage depths of typically 70-1000× redundancy. Alternatively or additionally, in some embodiments, the sequencing is any of the methods disclosed herein, as described in further detail below (see, e.g., the section entitled “Illustrative sequencing methods”). Thus, a first set of sequence reads is obtained.

In some embodiments, sequence reads are mapped (e.g., aligned) to a reference sequence 403. In some embodiments, the reference sequence is a human reference assembly. In some implementations, the mapping is performed using any suitable fast short read alignment software, such as Burrows-Wheeler alignment (BWA; see, e.g., Li and Durbin, Bioinformatics, 25:1754-60 (2009)). Details about BWA software and other alignment applications contemplated for use in the present disclosure are discussed in detail below (see, e.g., the section entitled “Alignment to reference sequence”). In some embodiments, duplicate reads produced by amplification steps in the library preparation are marked and/or removed to avoid double counting them. Any suitable preprocessing of sequence reads is contemplated for use in the present disclosure, as will be apparent to one skilled in the art.

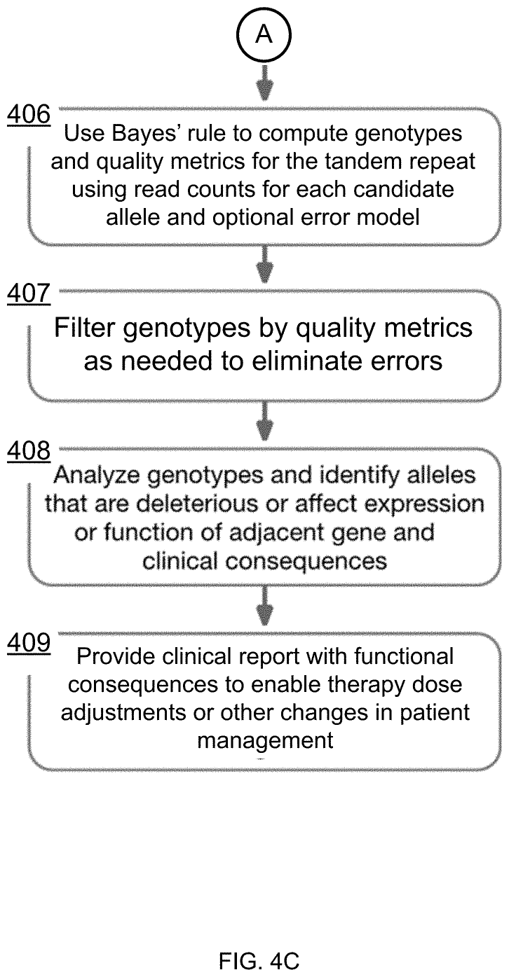

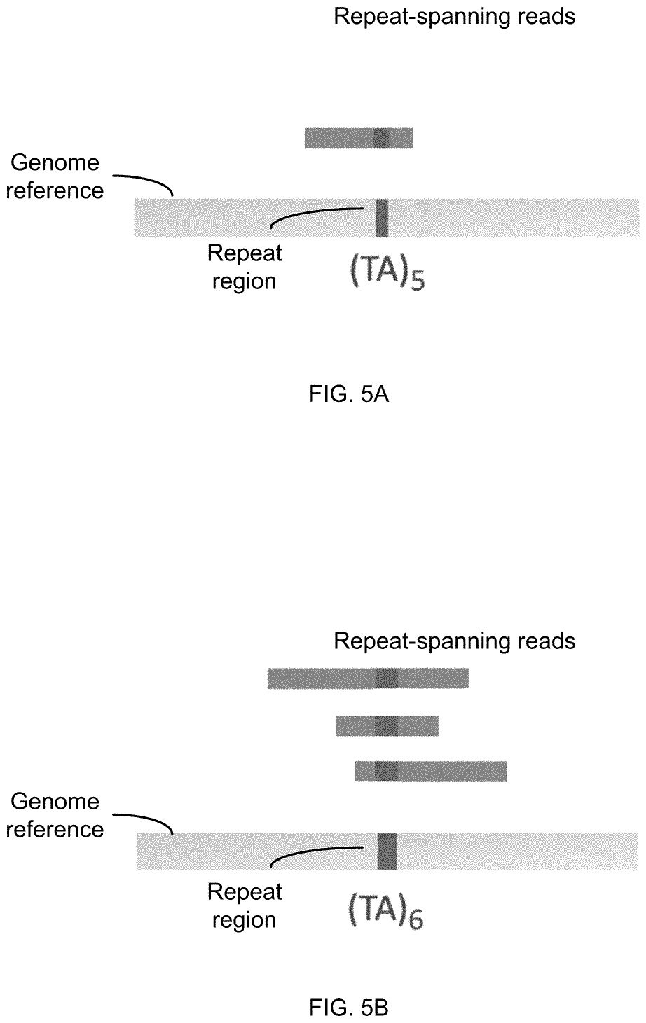

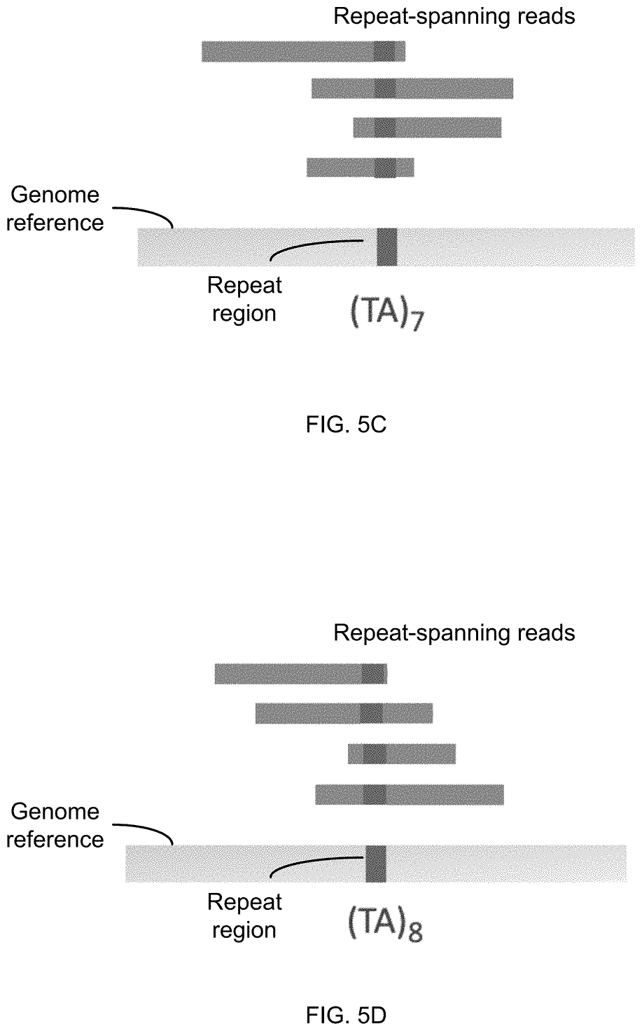

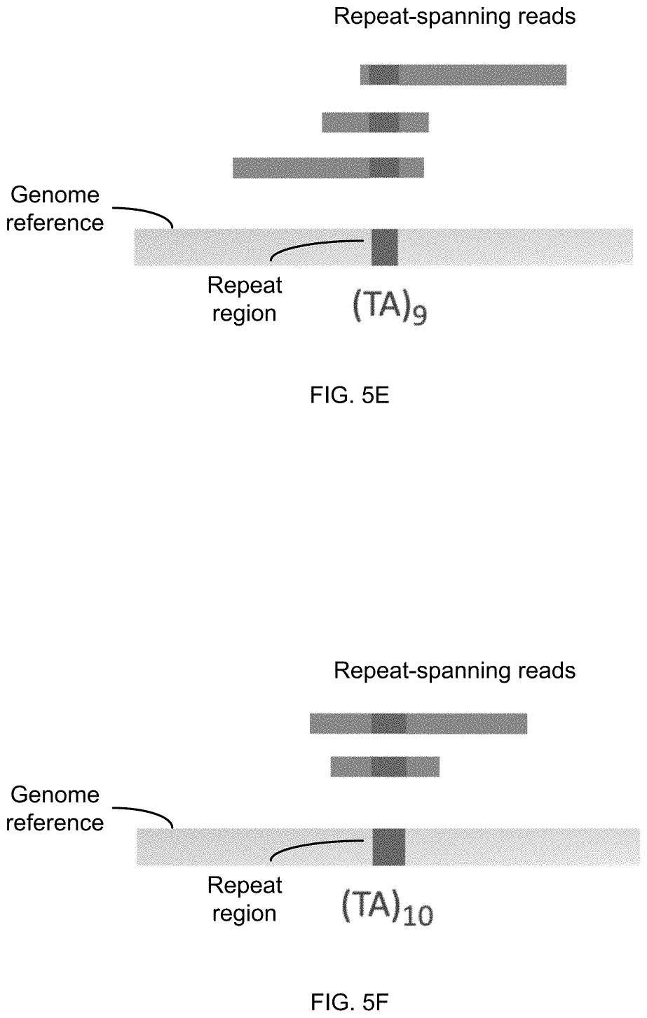

In some embodiments, sequence reads that span the repeat sequence of interest (e.g., the tandem repeat) are realigned 404 to a set of linear graph models that collectively represent a plurality of different repeat counts of the number of repeat units in the tandem repeat, such as the possible repeat lengths that are present in a reference population (e.g., the human population) and/or that are of medical or clinical interest (e.g., for UGT1A1, repeats of (TA)₅, (TA)₆, (TA)₇, (TA)₈, (TA)₉, and (TA)₁₀). In some such embodiments, each respective repeat length corresponds to a respective repeat count of the number of repeat units in the repeat sequence (e.g., the tandem repeat). Exemplary repeat counts for repeat sequences of interest represented by linear graph models are illustrated in FIGS. 5A-F, for repeat counts of 5, 6, 7, 8, 9, and 10.

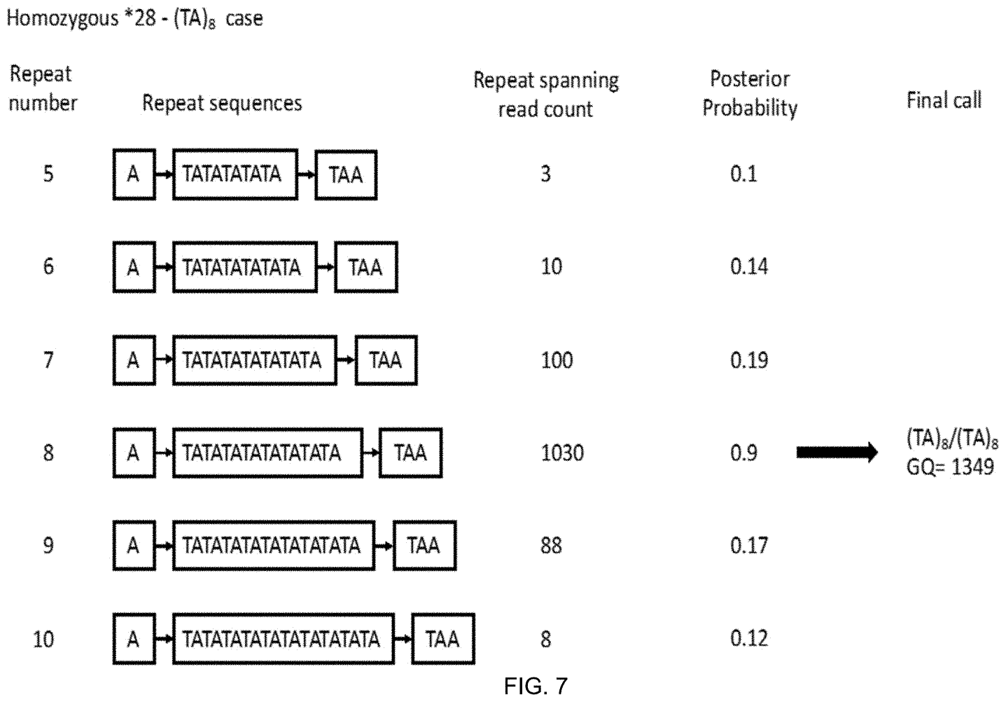

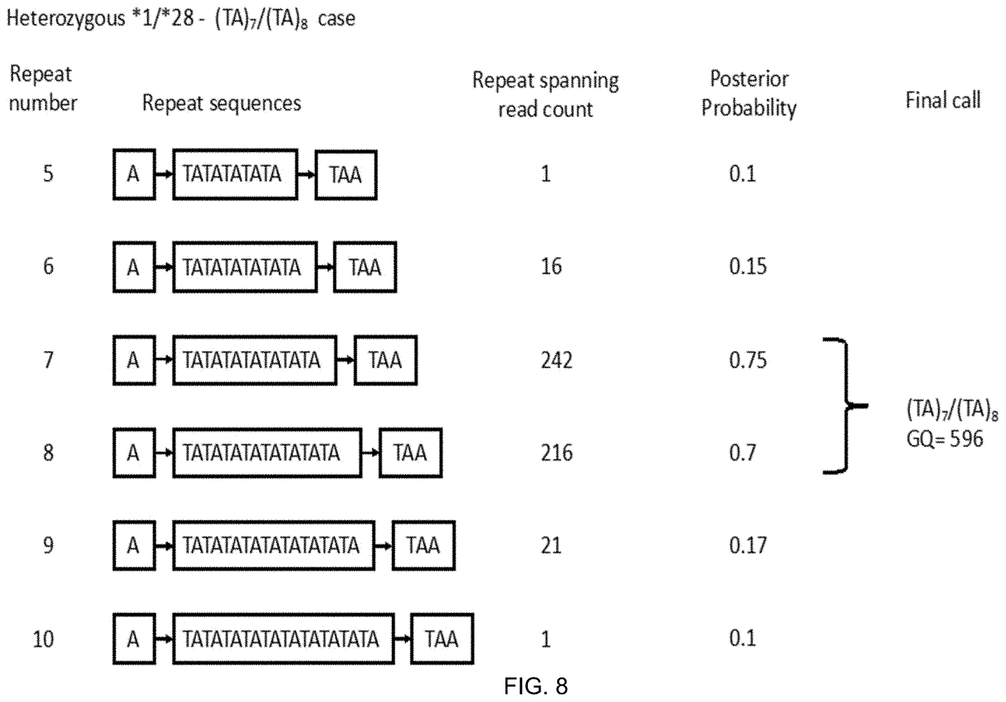

In some embodiments, for each respective repeat count in the plurality of different repeat counts (e.g., represented by the set of linear graph models), a corresponding number of sequence reads having the respective repeat count is determined (e.g., the number of sequence reads that map to a linear graph model corresponding to the respective repeat count). In some such embodiments, sequence reads are counted with respect to each of the possible repeat counts (e.g., possible repeat polymorphism lengths) expected in the reference population. In some embodiments, sequence reads are counted with respect to each respective candidate allele in a plurality of candidate alleles having different corresponding numbers of repeat units in the tandem repeat 405. An exemplary performance of this step is illustrated in FIGS. 7 and 8 for two different genotype outcomes. FIG. 7 provides results for a homozygous genotype of a TA repeat count of 8 for the UGT1A1 promoter region. FIG. 8 provides results for a heterozygous genotype of one TA repeat count of 7 and one TA repeat count of 8 for the UGT1A1 promoter region. In both illustrations, each respective repeat count in the set of possible repeat counts is shown in the left two columns (“Repeat number” and “Repeat sequences”) and the corresponding number of sequences having the respective repeat count (e.g., the number of sequence reads that map to a linear graph model corresponding to the respective repeat count) is shown in the middle column (“Repeat spanning read count”).

In some embodiments, the illustrative workflow further comprises applying a variant caller to the count of sequence reads corresponding to each respective possible repeat count in the set of possible repeat counts. For instance, in some embodiments, the workflow further comprises 406 using Bayes' rule to compute a genotype at the genomic locus comprising the tandem repeat, and one or more quality metrics thereof, using at least (i) the sequence read counts for each respective candidate allele in the plurality of candidate alleles having different corresponding numbers of repeat units in the tandem repeat and, optionally, (ii) an error model that models stutter (e.g., stutter is described in the section entitled “Introduction,” above). In some embodiments, the variant caller is based on Bayes' theorem (hereinafter, a “Bayesian variant caller”). In brief, Bayes' Theorem provides a principled way to calculate a conditional probability. In some implementations, the present systems and methods apply this theorem to the probability that a particular repeat sequence genotype is present in the sample of the subject, based on the first set of sequence reads. Callers are well known in the art and can be developed around various computational parameters that associate the data displayed by a particular set of reads with a particular genotype (see, e.g., Koboldt, Genome Med., 12:91 (2020)). Variant callers for determining genotypes for repeat sequences are described in greater detail below (see, e.g., the sections entitled “Assigning likelihood of candidate genotypes” and “Adjustment factors”).

In some embodiments, the variant caller allows for the generation of one or more genotype calls and/or quality metrics. For example, in some embodiments, a respective quality metric is a confidence value. In some embodiments, a respective quality metric is genotype quality or GQ. In various embodiments, the variant caller is any method or system used to distinguish and detect the alleles present in a specimen. In one example, as described above, the caller is a Bayesian caller. In another example, the caller is a machine learning or deep learning caller based on a machine learning algorithm trained on example data (e.g., DeepVariant). Machine learning algorithms for variant calling are known in the art. See, for example, Poplin et al., “A universal SNP and small-indel variant caller using deep neural networks,” Nature Biotechnology 36, 983-987 (2018); doi: 10.1038/nbt.4235.

In some embodiments, the illustrative workflow further includes obtaining a plurality of sets of repeat count adjustment factors. In some such embodiments, each respective set of repeat count adjustment factors corresponds to a candidate allele in a plurality of candidate alleles, where each respective candidate allele in the plurality of candidate alleles has a different corresponding number of repeat units for repeat sequence (e.g., each respective candidate allele is characterized by a different possible repeat length). Moreover, in some embodiments, each respective set of repeat count adjustment factors includes a corresponding repeat count adjustment factor for each respective number of repeat units in a numerical range of repeat units (e.g., a range of possible repeat counts within a given sequence read). Illustrative repeat count adjustment factors for a plurality of candidate alleles (“h”) and for a numerical range of repeat units (“r”) are shown below in Table 3 (see, e.g., the sections entitled “Assigning likelihood of candidate genotypes” and “Adjustment factors”).

In some embodiments, the applying the variant caller comprises counting alignment distributions manually within a reference data set, thereby obtaining the plurality of sets of repeat count adjustment factors. In some embodiments, the applying the variant caller comprises counting alignment distributions manually to remove stutter. In some embodiments, the variant caller optionally includes an error model 406 for determining alignment distributions.

In some embodiments, one or more genotype calls are filtered based on one or more quality metrics (e.g., confidence values) 407. In some such embodiments, the filtering removes one or more genotype calls of poor quality, such as those resulting from low-quality specimens and/or other experimental errors. In some implementations, the illustrative workflow further comprises evaluation of the one or more genotype calls, thereby determining a genotype of the subject for the genomic locus comprising the repeat sequence. In some embodiments, the evaluation includes review of the resulting genotype calls and/or identification of those for which phenotype or clinical implications have been described 408. In some implementations, this process is automated and includes cross-referencing with a database of previously known alleles (e.g., of functional, pharmacogenetics, and/or medical relevance).

In some embodiments, a report is provided 409, including any functional, medical, and/or pharmacogenetic data of relevance. For instance, in some such embodiments, the illustrative workflow includes providing a report to clinical personnel, where the report comprises the functional, medical, and/or pharmacogenetic relevance of the subject's repeat alleles to enable change in management, such as drug dose adjustments, or other remedies or changes as needed. In various embodiments, monitoring or dose regimens for patients having one or more genotype calls are selected according to established clinical guidelines for such subjects. Example monitoring schedules and dose regimens recommended for various genotype calls are known in the art. For instance, various studies of the UGT1A1 gene have reported maximum tolerated doses for irinotecan of 220 mg/m², 90 mg/m², or 75 mg/m² for the *28/*28 genotype, 150 mg/m² or 240 mg/m² for the *6 and *28 homozygous genotypes, and 210 mg/m² for the *28/*28 genotype. In some embodiments, recommended monitoring and dose regimens are based on one or more characteristics of the subject or a sample therefrom, such as cancer type, surgery status, metastatic status, histological features, ethnicity, and/or medication. Non-limiting examples of therapeutic regimens recommended for various genotypes are further described, for instance, in Argevani et al., “Dosage adjustment of irinotecan in patients with UGT1A1 polymorphisms: a review of current literature.” Innov Pharm. 2020; 11(3): 10.24926/iip.v11i3.3203; and Hulshof et al., “Pre-therapeutic UGT1A1 genotyping to reduce the risk of irinotecan-induced severe toxicity: Ready for prime time.” Eur J Cancer. 2020; 141: 9-20, each of which is hereby incorporated herein by reference in its entirety.

While FIGS. 4A-C depict representative workflows for determining repeat sequence genotypes, other implementations are possible, as will be apparent to one skilled in the art, and as will now be disclosed with reference to FIGS. 3A-E.

3. Example Methods

Referring to FIGS. 3A-E, one aspect of the present disclosure provides a method 300 of determining a genotype of a subject at a genomic locus comprising a tandem repeat, from a plurality of candidate genotypes for the genomic locus. In some embodiments, the method is performed at a computer system having one or more processors and memory storing at least one program for execution by the one or more processors.

Referring to Block 302, in some embodiments, the genomic locus is a gene. Referring to Block 304, in some such embodiments, the tandem repeat is in a promoter of the gene. Referring to Block 306, in some embodiments, the gene is the UDP glucuronosyltransferase family 1 member A1 (UGT1A1) gene. In some implementations, the genomic locus is any of the genomic loci disclosed herein. In some implementations, the tandem repeat is any of the repeat sequences disclosed herein. For instance, non-limiting genomic loci and tandem repeats are described in further detail in the section entitled “Example repeat sequences,” above.

Referring to Block 308, in some embodiments, the plurality of candidate alleles comprises a first allele comprising an A(TA)₆TAA TATA box, a second allele comprising an A(TA)₇TAA TATA box, a third allele comprising an A(TA)₈TAA TATA box, and a fourth allele comprising an A(TA)₉TAA TATA box. For instance, in some such embodiments, the genomic locus is the UDP glucuronosyltransferase family 1 member A1 (UGT1A1) gene, and the tandem repeat is in a promoter of the gene.

In some embodiments, the nucleotide repeat unit is from 2 to 6 nucleotides long.

In some embodiments, the nucleotide repeat unit is at least 2, at least 3, at least 4, at least 5, at least 6, at least 7, at least 8, at least 9, at least 10, at least 15, at least 20, or at least 30 nucleotides long. In some embodiments, the nucleotide repeat unit is no more than 50, no more than 30, no more than 20, no more than 10, or no more than 5 nucleotides long. In some embodiments, the nucleotide repeat unit is from 2 to 10, from 2 to 5, from 3 to 15, from 12 to 25, or from 20 to 50 nucleotides long. In some embodiments, the nucleotide repeat unit has a length that falls within another range starting no lower than 2 nucleotides and ending no higher than 50 nucleotides.

In some embodiments, the tandem repeat has from 2 to 100 contiguous nucleotide repeat units.

In some embodiments, the tandem repeat has at least 2, at least 3, at least 4, at least 5, at least 6, at least 7, at least 8, at least 9, at least 10, at least 15, at least 20, at least 30, at least 50, at least 80, at least 100, at least 200, or at least 300 contiguous nucleotide repeat units. In some embodiments, the tandem repeat has no more than 500, no more than 300, no more than 200, no more than 100, no more than 50, no more than 20, no more than 10, or no more than 5 contiguous nucleotide repeat units. In some embodiments, the tandem repeat has from 2 to 14, from 5 to 10, from 6 to 9, from 3 to 20, from 10 to 50, from 30 to 100, from 80 to 200, or from 100 to 500 contiguous nucleotide repeat units. In some embodiments, the tandem repeat has a number of contiguous nucleotide repeat units that falls within another range starting no lower than 2 repeat units and ending no higher than 500 repeat units.

In some embodiments, the tandem repeat has from 2 to 10 contiguous nucleotide repeat units.

Referring to Block 310, in some embodiments, expansion or contraction of the tandem repeat is linked with a change in a drug metabolism.

Referring to Block 312, in some embodiments, expansion or contraction of the tandem repeat is linked to a disorder.

In some embodiments, the genomic locus is a gene selected from the group consisting of FMR1 (Fragile X syndrome), PPP2R2B (Spinocerebellar ataxia 12), ATXN1 (Spinocerebellar ataxia 1), ATXN2 (Spinocerebellar ataxia 2), ATXN3 (Spinocerebellar ataxia 3), CACNA1A (Spinocerebellar ataxia 6), ATXN7 (Spinocerebellar ataxia 7), (HTT) Huntington's disease, AR (Spinal and bulbar muscular atrophy), ATN1 (Dentatorubral-pallidoluysian atrophy), FXN (Friedreich's ataxia), CNBP (Myotonic dystrophy 2), ATXN10 (Spinocerebellar ataxia 10), BEAN1 (Spinocerebellar ataxia 31), NOP56 (Spinocerebellar ataxia 36), C9ORF72 (Amyotrophic lateral sclerosis), COMP (multiple skeletal dysplasias), HOXD13 (Synpolydactyly syndrome), HOXA13 (Hand-foot-genital syndrome), RUNX2 (Cleidocranial dysplasia), ZIC2 (Holoprosencephaly), PABPN1 (Oculopharyngeal muscular atrophy), FOXL2 (Blepharophimosis, ptosis, epicanthus inversus syndrome), ARX (ARX-related X-linked mental retardation), DMPK (Myotonic dystrophy 1), ATXN8OS (Spinocerebellar ataxia 8), JPH3 (Huntington's disease-like 2), CSTB (Myoclonic epilepsy of Unverricht and Lundborg), and TBP (Spinocerebellar ataxia 17). See, for example, Sun et al., “Disease-Associated Short Tandem Repeats Co-localize with Chromatin Domain Boundaries,” Cell, 175(1), 2018, 224-238.e15, which is hereby incorporated herein by reference in its entirety.

In some embodiments, the plurality of candidate genotypes comprises each combination of two respective candidate alleles in a plurality of candidate alleles.

In some embodiments, the plurality of candidate alleles comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, or at least 100 alleles. In some embodiments, the plurality of candidate alleles comprises no more than 200, no more than 100, no more than 50, no more than 30, no more than 20, no more than 10, no more than 8, or no more than 5 alleles. In some embodiments, the plurality of candidate alleles consists of from 2 to 10, from 2 to 20, from 2 to 30, from 3 to 12, from 2 to 14, from 5 to 10, from 6 to 9, from 3 to 20, from 10 to 50, from 30 to 100, or from 80 to 200 alleles. In some embodiments, the plurality of candidate alleles falls within another range starting no lower than 2 alleles and ending no higher than 200 alleles.

In some embodiments, each respective candidate allele in the plurality of candidate alleles has a different repeat count of the number of repeat units in the tandem repeat. Accordingly, in some such embodiments, each respective candidate allele in the plurality of candidate alleles is for a different corresponding length of the tandem repeat.

In some embodiments, the plurality of candidate alleles represents a plurality of different repeat counts of the number of repeat units in the tandem repeat (e.g., where each respective repeat count in the plurality of different repeat counts is the repeat count of at least one respective candidate allele in the plurality of candidate alleles). In some embodiments, the plurality of candidate alleles represents a numerical range of different repeat counts of the number of repeat units in the tandem repeat.

In some embodiments, the plurality of different repeat counts represented by the plurality of candidate alleles includes all of the possible repeat counts within a particular numerical range. For example, in some such embodiments, for a numerical range of repeat counts consisting of 2 to 5 repeat units in the tandem repeat, the plurality of candidate alleles would consist of a candidate allele having a repeat count of 2, a candidate allele having a repeat count of 3, a candidate allele having a repeat count of 4, and a candidate allele having a repeat count of 5.

In some embodiments, the plurality of different repeat counts represented by the plurality of candidate alleles is noncontiguous, such that one or more repeat counts within a particular numerical range is not represented within the plurality of candidate alleles. For example, in some such embodiments, for a numerical range of repeat counts consisting of 2 to 5 repeat units in the tandem repeat need not include all of the possible repeat unit counts within the range of 2 to 5. Thus, in an embodiment, the plurality of candidate alleles would consist of a candidate allele having a repeat count of 2, a candidate allele having a repeat count of 4, and a candidate allele having a repeat count of 5.

In some embodiments, the plurality of different repeat counts of the number of repeat units in the tandem repeat represented in the plurality of candidate alleles has a lower bound of at least 2, at least 3, at least 4, at least 5, at least 6, at least 7, at least 8, at least 9, at least 10, at least 15, at least 20, at least 30, at least 50, at least 80, at least 100, at least 200, or at least 300 repeat units. In some embodiments, the plurality of different repeat counts of the number of repeat units in the tandem repeat represented in the plurality of candidate alleles has an upper bound of no more than 500, no more than 300, no more than 200, no more than 100, no more than 50, no more than 20, no more than 10, or no more than 5 repeat units. In some embodiments, the plurality of different repeat counts of the number of repeat units in the tandem repeat represented in the plurality of candidate alleles ranges from 2 to 14, from 2 to 10, from 5 to 10, from 6 to 9, from 3 to 20, from 10 to 50, from 30 to 100, from 80 to 200, or from 100 to 500 repeat units. In some embodiments, the plurality of different repeat counts of the number of repeat units in the tandem repeat represented in the plurality of candidate alleles falls within another range starting no lower than 2 repeat units and ending no higher than 500 repeat units.

In some embodiments, the number of candidate genotypes in the plurality of candidate genotypes is from 3 to 210.

In some embodiments, the plurality of candidate genotypes comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 100, at least 150, at least 200, at least 500, at least 1000, or at least 2000 genotypes. In some embodiments, the plurality of candidate genotypes comprises no more than 5000, no more than 2000, no more than 1000, no more than 500, no more than 300, no more than 200, no more than 100, no more than 50, no more than 20, or no more than 10 genotypes. In some embodiments, the plurality of candidate genotypes consists of from 3 to 210, from 3 to 500, from 10 to 250, from 10 to 80, from 5 to 25, from 40 to 100, or from 500 to 2000 genotypes. In some embodiments, the plurality of candidate genotypes falls within another range starting no lower than 3 genotypes and ending no higher than 5000 genotypes.

For instance, consider an illustrative example in which the plurality of candidate alleles consists of 20 possible alleles (e.g., haplotypes). In some embodiments where the plurality of candidate genotypes comprises all possible combinations of two respective candidate alleles in the plurality of candidate alleles, and taking conditions of both heterozygosity and homozygosity into account, the total number of candidate genotypes that can be generated from the plurality of 20 candidate alleles is 20+19+18+17+16+15+14+13+12+11+10+9+8+7+6+5+4+3+2+1=210.

Referring to Block 314, the method 300 comprises obtaining, in electronic form, a first set of sequence reads 124 obtained from a biological sample of the subject that map to the tandem repeat in the genomic locus, where the tandem repeat consists of a plurality of contiguous nucleotide repeat units and each respective sequence read in the first set of sequence reads encompasses the tandem repeat. In some embodiments, the first set of sequence reads is deduplicated. For example, in some embodiments, each respective sequence read in the first set of sequence reads has a unique identity. For instance, in some embodiments, each respective sequence read in the first set of sequence reads corresponds to a unique nucleic acid molecule from which the respective sequence read is obtained (e.g., a unique molecular identifier or UMI).

In some embodiments, the first set of sequence reads comprises at least 25, at least 50, at least 100, at least 500, at least 1000, at least 5000, at least 1×10⁴, at least 5×10⁴, at least 1×10⁵, at least 5×10⁵, at least 1×10⁶, at least 5×10⁶, at least 1×10⁷, or at least 5×10⁷ sequence reads. In some embodiments, the first set of sequence reads comprises no more than 1×10⁸, no more than 1×10⁷, no more than 1×10⁶, no more than 1×10⁵, no more than 1×10⁴, no more than 1000, or no more than 500 sequence reads. In some embodiments, the first set of sequence reads consists of from 25 to 500, from 100 to 1000, from 200 to 5000, from 1000 to 1×10⁴, from 1×10⁴ to 5×10⁵, from 1×10⁵ to 1×10⁶, from 1×10⁶ to 1×10⁸, or from 5×10⁶ to 1×10⁷ sequence reads. In some embodiments, the first set of sequence reads falls within another range starting no lower than 25 sequence reads and ending no higher than 1×10⁸ sequence reads.

In some embodiments, the first set of sequence reads is at least 25 sequence reads, at least 100 sequence reads, at least 1000 sequence reads, or at least 10,000 sequence reads.

In some embodiments, the first set of sequence reads is a sub-plurality of a first plurality of sequence reads. In some embodiments, the first plurality of sequence reads is at least 100,000 sequence reads, at least 500,000 sequence reads, or at least 1,000,000 sequence reads. In some embodiments, the first plurality of sequence reads comprises at least 25, at least 50, at least 100, at least 500, at least 1000, at least 5000, at least 1×10⁴, at least 5×10⁴, at least 1×10⁵, at least 5×10⁵, at least 1×10⁶, at least 5×10⁶, at least 1×10⁷, or at least 5×10⁷ sequence reads. In some embodiments, the first plurality of sequence reads comprises no more than 1×10⁸, no more than 1×10⁷, no more than 1×10⁶, no more than 1×10⁵, no more than 1×10⁴, no more than 1000, or no more than 500 sequence reads. In some embodiments, the first plurality of sequence reads consists of from 25 to 500, from 100 to 1000, from 200 to 5000, from 1000 to 1×10⁴, from 1×10⁴ to 5×10⁵, from 1×10⁵ to 1×10⁶, from 1×10⁶ to 1×10⁸, or from 5×10⁶ to 1×10⁷ sequence reads. In some embodiments, the first plurality of sequence reads falls within another range starting no lower than 25 sequence reads and ending no higher than 1×10⁸ sequence reads.

In some embodiments, the first plurality of sequence reads is deduplicated. In some embodiments, each respective sequence read in the first plurality of sequence reads has a unique identity. For instance, in some embodiments, each respective sequence read in the first plurality of sequence reads corresponds to a unique nucleic acid molecule from which the sequence read is obtained (e.g., a unique molecular identifier or UMI). In some embodiments, the first plurality of sequence reads is obtained using any of the methods disclosed herein. See, for example, the sections entitled “Nucleic acid extraction from biological sample,” “Sequencing library preparation,” and “Illustrative sequencing methods,” below.

Referring to Block 316, in some embodiments, the biological sample of the subject is a non-cancerous tissue sample of the subject. In some embodiments, the biological sample of the subject is a solid tissue sample. In some embodiments, the biological sample of the subject is a liquid biopsy sample. In some embodiments, the liquid biopsy sample is a blood sample, urine sample, or saliva sample. Non-limiting examples of biological samples and subjects contemplated for use in the present disclosure are further disclosed in the section entitled “Subjects and samples,” below.

Referring to Block 318, the method further includes determining, for each respective sequence read 124 in the first set of sequence reads, a corresponding repeat count 126 of the number of repeat units in the plurality of contiguous repeat units in the respective sequence read, thereby determining a distribution of repeat counts of the number of repeat units in the first set of sequence reads.

Various methods of obtaining repeat counts for sequence reads, and/or distributions of repeat counts thereof, are contemplated for use in the present disclosure.

For example, in some embodiments, the method includes performing at least a first mapping (e.g., a first alignment) of a plurality of sequence reads including the first set of sequence reads to a reference genome, and optionally performing at least a second mapping (e.g., a second alignment) of all or a portion of the plurality of sequence reads to a linear graph model that represents a plurality of candidate alleles having different repeat counts for the tandem repeat.

Thus, referring to Block 320, in some embodiments, the obtaining the first set of sequence reads comprises sequencing a first plurality of nucleic acids from the biological sample of the subject, thereby obtaining a first plurality of sequence reads that comprises the first set of sequence reads, and mapping the first plurality of sequence reads against a genomic reference construct comprising the tandem repeat, thereby identifying a first sub-plurality of the first plurality of sequence reads that map to a genomic position within a threshold distance from the tandem repeat in the genomic reference construct.

In some embodiments, the first plurality of nucleic acids from the biological sample has been enriched for nucleic acids comprising the tandem repeat.

In some embodiments, the mapping comprises a global alignment of the first plurality of sequence reads to the genomic reference construct (e.g., a Burrows-Wheeler alignment (BWA)). Non-limiting examples of mapping methods and algorithms contemplated for use in the present disclosure are further described in the section entitled “Alignment to reference genome,” below.

In some embodiments, the threshold distance from the tandem repeat is no more than 5 kb, no more than 1 kb, or no more than 250 bp.

In some embodiments, the threshold distance from the tandem repeat is no more than 10 kb, no more than 5 kb, no more than 2 kb, no more than 1 kb, no more than 500 bp, no more than 250 bp, no more than 100 bp, or no more than 50 bp. In some embodiments, the threshold distance from the tandem repeat is at least 10 bp, at least 50 bp, at least 100 bp, at least 250 bp, at least 1 kb, or at least 5 kb. In some embodiments, the threshold distance from the tandem repeat is from 10 bp to 500 bp, from 100 bp to 1 kb, from 500 bp to 5 kb, or from 2 kb to 10 kb. In some embodiments, the threshold distance from the tandem repeat falls within another range starting no lower than 10 bp and ending no higher than 10 kb.

Referring to Block 322, in some embodiments, the obtaining the first set of sequence reads further comprises aligning the first sub-plurality of the first plurality of sequence reads against a plurality of reference structures for the genomic locus, where each respective reference structure in the plurality of reference structures comprises a different repeat count of the number of repeat units in the tandem repeat; and the determining comprises counting, for each respective reference structure in the plurality of reference structures, a corresponding number of sequence reads in the first set of sequence reads that map to the respective reference structure.

Referring to Block 324, in some embodiments, the plurality of reference structures is a linear graph model. For example, in some such embodiments, the plurality of reference structures is a linear graph model for the plurality of candidate alleles, where each respective reference structure represents a corresponding candidate allele having a different repeat count of the number of repeat units in the tandem repeat (e.g., a respective haploid repeat sequence genotype).

In other words, in some such embodiments, the determining the distribution of repeat counts of the number of repeat units in the first set of sequence reads includes counting the number of alignments of sequence reads to each haploid genotype represented in a linear graph model.

In some embodiments, the number of reference structures in the plurality of reference structures is equal to the number of candidate alleles in the plurality of candidate alleles (e.g., where each respective candidate allele in the plurality of candidate alleles has a different repeat count of the number of repeat units in the tandem repeat).

In some embodiments, the plurality of reference structures comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, or at least 100 reference structures. In some embodiments, the plurality of reference structures comprises no more than 200, no more than 100, no more than 50, no more than 30, no more than 20, no more than 10, no more than 8, or no more than 5 reference structures. In some embodiments, the plurality of reference structures consists of from 2 to 10, from 2 to 20, from 2 to 30, from 3 to 12, from 2 to 14, from 5 to 10, from 6 to 9, from 3 to 20, from 10 to 50, from 30 to 100, or from 80 to 200 reference structures. In some embodiments, the plurality of reference structures falls within another range starting no lower than 2 reference structures and ending no higher than 200 reference structures.

Alternatively or additionally, referring to Block 326, in some embodiments, the determining comprises counting, for each respective repeat count of the number of repeat units in the tandem repeat in a plurality of repeat counts of the number of repeat units in the tandem repeat, the number of sequence reads in the first sub-plurality of sequence reads having the respective repeat count of the number of repeat units in the tandem repeat.

For instance, in some such embodiments, the determining the distribution of repeat counts includes, for each respective repeat count in a plurality of repeat counts observed in the first set of sequence reads, using the corresponding repeat count for each respective sequence read in the first set of sequence reads to obtain a tally (e.g., a simple count) of sequence reads having the respective repeat count. In some such embodiments, the determining the distribution of repeat counts is performed without a first alignment to a genomic reference construct comprising the tandem repeat. Alternatively or additionally, in some embodiments, the determining the distribution of repeat counts is performed without a second alignment to a plurality of reference structures.

Suitable methods for determining distributions of repeat counts that are contemplated for use in the present disclosure are further described in the section entitled “Assigning likelihood of candidate genotypes,” above.

Referring to Block 328, the method further includes obtaining a plurality of sets of repeat count adjustment factors 134, where each respective set of repeat count adjustment factors corresponds to a candidate allele 132 in a plurality of candidate alleles, each respective candidate allele in the plurality of candidate alleles has a different corresponding number of repeat units 136 for the plurality of contiguous nucleotide repeat units, each respective set of repeat count adjustment factors includes a corresponding repeat count adjustment factor for each respective number of repeat units in a numerical range 138 of repeat units for the plurality of contiguous nucleotide repeat units, and each combination of two respective candidate alleles 132 in the plurality of candidate alleles corresponds to a respective candidate genotype 144 in the plurality of candidate genotypes.

In some implementations, the plurality of candidate alleles comprises any of the ranges and/or embodiments disclosed above. Thus, in some embodiments, each respective candidate allele in the plurality of candidate alleles is for a different corresponding length of the tandem repeat, as described above (e.g., a different repeat count of the number of repeat units in the tandem repeat), and each respective set of repeat count adjustment factors corresponds to a different candidate allele having a different repeat count.

An example of a plurality of sets of repeat count adjustment factors is provided below in Table 3. For each respective candidate allele in a plurality of candidate alleles (h=6, h=7, h=8, and h=9), a corresponding set of repeat count adjustment factors is shown in the accompanying column. In some such embodiments, each respective set of repeat count adjustment factors includes a respective repeat count adjustment factor for each respective number of repeat units (r=2, r=3, r=4, . . . r=12) in a numerical range of repeat units (e.g., range of 2 to 12). Thus, the plurality of sets of repeat count adjustment factors, in some implementations, is represented as a matrix, with each respective repeat count adjustment factor corresponding to a respective candidate allele in the plurality of candidate alleles (e.g., columns) and a respective number of repeat units in a numerical range (e.g., rows).

In some embodiments, the numerical range is from 2 to 12. In some embodiments, the numerical range comprises the number of contiguous nucleotide repeat units represented in the plurality of candidate alleles. In some embodiments, the numerical range comprises a number of contiguous nucleotide repeat units that are observed or expected to be observed within the first set of sequence reads. In some embodiments, the numerical range further comprises one or more numbers of repeat units that are not observed or not expected to be observed within the first set of sequence reads. In some such embodiments, the numerical range extends beyond the upper and/or lower bounds of the range of observable repeat counts in the first set of sequence reads.

In some embodiments, the numerical range has a lower bound of at least 2, at least 3, at least 4, at least 5, at least 6, at least 7, at least 8, at least 9, at least 10, at least 15, at least 20, at least 30, at least 50, at least 80, at least 100, at least 200, or at least 300. In some embodiments, the numerical range has an upper bound of no more than 500, no more than 300, no more than 200, no more than 100, no more than 50, no more than 20, no more than 10, or no more than 5. In some embodiments, the numerical range is from 2 to 14, from 2 to 10, from 5 to 10, from 6 to 9, from 3 to 20, from 10 to 50, from 30 to 100, from 80 to 200, or from 100 to 500. In some embodiments, the numerical range is another range starting no lower than 2 and ending no higher than 500.

Referring to Block 330, in some embodiments, a respective repeat count adjustment factor in a respective set of repeat count adjustment factors in the plurality of sets of repeat count adjustment factors is determined based on a proportion of sequence reads, in a second plurality of sequence reads obtained from a reference sample, having a respective repeat count of the number of repeat units in the tandem repeat, where the reference sample comprises polynucleotides encompassing the tandem repeat having a known respective repeat count of the number of repeat units in the tandem repeat.

In some such embodiments, the reference sample is a biological reference sample. In some embodiments, the reference sample is a plurality of biological reference samples. In some embodiments, the reference sample is a synthetic reference sample. In some embodiments, the reference sample is a plurality of synthetic reference samples.

In some embodiments, the plurality of sets of repeat count adjustment factors is an empirically derived distribution of repeat counts of the number of repeat units in the tandem repeat observed in the second plurality of sequence reads obtained from the reference samples. For instance, empirically derived distributions of repeat counts, and methods of obtaining the same, are further described in the sections entitled “Assigning likelihood of candidate genotypes” and “Adjustment factors,” below.

Referring to Block 332, in some embodiments, a respective repeat count adjustment factor in a respective set of repeat count adjustment factors in the plurality of sets of repeat count adjustment factors is determined using an error model.

In some embodiments, the error model has the formula:

$$p ( 0 \vert 0 ) = 1 , \qquad [ Equation 1 ]$$

$$p ( r \vert h ) = ( 1 - s ) p ( r - 1 \vert h - 1 ) + \frac{s}{2} p ( r - 2 \vert h - 1 ) + \frac{s}{2} p ( r \vert h - 1 ) ,$$

for 0≤r≤2h and 0 elsewhere, where:

p(r|h) is a probability of observing r repeat units in a sequence read obtained from a respective polynucleotide having h repeat units; and

s is a probability that a respective repeat unit will be duplicated or deleted during sequencing of the respective polynucleotide. Note:

$${\sum }_{r = 0}^{2 h} p ( r \vert h ) = 1 .$$

In some embodiments, the error model is a simple, one-parameter error model. Error models contemplated for use in the present disclosure are further described herein (see, e.g., the section entitled “Adjustment factors,” below).

Referring to Block 334, the method further comprises assigning, for each respective candidate genotype 144 in the plurality of candidate genotypes, a corresponding likelihood 142 for the respective candidate genotype based, at least in part, upon, for each respective candidate allele 132 corresponding to the respective candidate genotype: (i) a proportion of sequence reads 124 in the plurality of sequence reads that have the repeat count 126 of the number of repeat units in the plurality of contiguous repeat units corresponding to the respective candidate allele, and (ii) a repeat count adjustment factor 134 matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles.

For example, in some embodiments, a corresponding likelihood for a respective candidate genotype is determined by taking into account the probabilities for each of the candidate alleles within the respective candidate genotype. In some such embodiments, the probabilities for each respective candidate allele is determined using a respective proportion (e.g., frequency or count) of sequence reads in the first set of sequence reads that have the same repeat count as the respective candidate allele, which is further adjusted using the corresponding set of repeat count adjustment factors matching the respective candidate allele.

Referring to Block 336, in some embodiments, the assigning the corresponding likelihood for the respective candidate genotype is further based upon: for each respective candidate allele in the plurality of candidate allele that does not correspond to the respective candidate genotype: (i) a proportion of sequence reads in the plurality of sequence reads that have the repeat count of the number of repeat units in the plurality of contiguous repeat units corresponding to the respective candidate allele, and (ii) a repeat count adjustment factor matching the number of repeat units in the plurality of contiguous repeat units corresponding to the respective candidate allele from the set of repeat count adjustment factors for the respective candidate allele.

Thus, in some embodiments, the probabilities of observing, in the first set of sequence reads, repeat counts that do not match the corresponding alleles for the respective candidate genotype, are further accounted for in the probabilistic determination.

In some embodiments, the corresponding likelihood for the respective candidate genotype is determined according to:

$$P ( H \vert E ) = \frac{P ( E \vert H ) P ( H )}{P ( E )} , \qquad [ Equation 2 ]$$

where:

E represents the distribution of repeat counts of the number of repeat units in the first set of sequence reads

H represents a corresponding hypothesis that the subject has the respective candidate genotype for the genomic locus,

P(H) is a prior probability that the subject has the respective candidate genotype for the genomic locus,

P(E|H) is a conditional probability of observing the distribution of repeat counts of the number of repeat units in the first set of sequence reads if the subject has the respective candidate genotype for the genomic locus,

P(E) is a marginal probability of observing the distribution of repeat counts of the number of repeat units in the first set of sequence reads regardless of the subject's genotype for the genomic locus, and

P(H|E) is a posterior probability that the subject has the respective candidate genotype for the genomic locus given the distribution of repeat counts of the number of repeat units in the first set of sequence reads.

In some embodiments, for each respective candidate genotype in the plurality of candidate genotypes, the conditional probability is:

$$P ( E \vert H ) = {\prod }_{r} {P ( r \vert H )}^{{f}_{r}} , \qquad [ Equation 3 ]$$

where:

P(r|H) is the probability of observing r repeat units in a respective sequence read if the subject has the respective candidate genotype for the genomic locus, according to the formula:

$$P ( r \vert H ) = \{ \begin{matrix}\frac{1}{2} [ p ( r \vert a ) + p ( r \vert b ) ] & if a \neq b ( hete r o z y g ous ) \\ p ( r \vert a ) & if a = b ( homo z y gous )\end{matrix} ,$$

where:

when the candidate genotype for the genomic locus is a homozygous genotype for a candidate allele in the plurality of candidate alleles, P(r|H) is the repeat count adjustment factor, in the set of repeat count adjustment factors corresponding to the candidate allele, corresponding to r repeat units, and

when the candidate genotype for the genomic locus is a heterozygous genotype for a first candidate allele in the plurality of candidate alleles and a second candidate allele in the plurality of candidate alleles, P(r|H) is an arithmetic combination of (i) the repeat count adjustment factor, in the set of repeat count adjustment factors corresponding to the first candidate allele, corresponding to r repeat units, and (ii) the repeat count adjustment factor, in the set of repeat count adjustment factors corresponding to the second candidate allele, corresponding to r repeat units, and

fᵣ is a corresponding count of sequence reads, in the first plurality of sequence reads, having r repeat units.

In some embodiments, the conditional probability is determined in logarithmic space.

Non-limiting examples of suitable methods for determining conditional probabilities and/or corresponding likelihoods, including methods for determining P(r|H), contemplated for use in the present disclosure are further described in the section entitled “Assigning likelihood of candidate genotypes,” below.

In some embodiments, the assigning the corresponding likelihood for the respective candidate genotype further comprises determining one or more quality metrics for the respective candidate genotype. Referring to Block 338, in some embodiments, the assigning the corresponding likelihood for the respective candidate genotype further comprises determining a corresponding first quality metric for the respective candidate genotype. In some embodiments, a respective quality metric is a confidence value. In some embodiments, a respective quality metric is genotype quality or GQ. Non-limiting examples of quality metrics contemplated for use in the present disclosure are further described in Caetano-Anolles, “Calculation of PL and GQ by HaplotypeCaller and GenotypeGVCFs,” 2022, available on the Internet at gatk.broadinstitute.org/hc/en-us/articles/360035890451-Calculation-of-PL-and-GQ-by-HaplotypeCaller-and-GenotypeGVCFs, which is hereby incorporated herein by reference in its entirety. In some embodiments, a respective quality metric is a log-odds posterior probability. In some embodiments, the corresponding first quality metric for the respective candidate genotype is a log-odds ratio of the corresponding likelihood for the respective candidate genotype.

Referring to Block 340, in some embodiments, the method further comprises filtering the plurality of candidate genotypes based on the corresponding first quality metric by a procedure comprising: when the corresponding first quality metric satisfies a threshold quality metric score, retaining the respective candidate genotype in the plurality of candidate genotypes; and when the corresponding first quality metric fails to satisfy the threshold quality metric score, removing the respective candidate genotype from the plurality of candidate genotypes.

Referring to Block 342, the method further comprises selecting the respective candidate genotype 144 in the plurality of candidate genotypes having the highest corresponding likelihood 142. For instance, in some such embodiments, the respective candidate genotype is selected based on a maximum likelihood, in a plurality of corresponding likelihoods for the plurality of candidate genotypes.

Non-limiting examples of suitable methods for determining quality metrics and/or selecting candidate genotypes based on likelihood are further described in the section entitled “Assigning likelihood of candidate genotypes,” below.

Referring to Block 344, in some embodiments, the method further includes generating a report comprising at least (i) the respective candidate genotype in the plurality of candidate genotypes having the highest corresponding likelihood and (ii) the corresponding first quality metric for the respective candidate genotype in the plurality of candidate genotypes having the highest corresponding likelihood.

Referring to Block 346, in some embodiments, the report further comprises one or more of: a risk of adverse drug reaction for the subject, a risk of disease for the subject, a drug dosage recommendation for the subject, a drug prescription recommendation for the subject, a validation genotype of the subject for the genomic locus comprising the tandem repeat, a variant call for an auxiliary genomic region, other than the target genomic region, and a validation status for the respective candidate genotype in the plurality of candidate genotypes having the highest corresponding likelihood.

Non-limiting examples of suitable reports and methods of generating the same are further described in the section entitled “Further methods and applications,” below.

In some embodiments, the selecting the respective candidate genotype having the highest corresponding likelihood indicates a presence or absence of a repeat sequence polymorphism (e.g., a tandem repeat polymorphism) in the one or more alleles at the target genomic region.

In some embodiments, the method further comprises using the respective candidate genotype in the plurality of candidate genotypes having the highest corresponding likelihood to determine a risk of adverse drug reaction for a first chemotherapeutic drug.

In some implementations, the first chemotherapeutic drug is metabolized by the enzyme product of the UDP glucuronosyltransferase family 1 member A1 (UGT1A1) gene. In some implementations, the first chemotherapeutic drug is metabolized by the enzyme product of the dihydropyrimidine dehydrogenase (DPYD) gene.

In some embodiments, the first chemotherapeutic drug is selected from the group consisting of abiraterone, acalabrutinib, asciminib, anastrozole, axitinib, belinostat, bendamustin, bexarotene, bicalutamide, binimetinib, bleomycin, camptothecin, cerdulatinib, chlorambucil, cobimetinib, cytarabine, dasatinib, daunorubicin, doxorubicin, duvelisib, enasidenib, encorafenib, epirubicin, erlotinib, etoposide, exemestane, fenretinide, flavopiridol, fludarabine, 5-fluorouracil, fluoxymesterone, flumatinib, folfiri, folfox, fostamatinib, fulvestrant, glasdegib, govitecan, ibrutinib, idelalisib, imatinib, irinotecan, isotretinoin, larotrectinib, letrozole, lorlatinib, medroxyprogesterone, megestrol, methotrexate, mitoxantrone, nintedanib, niraparib, olaparib, palbociclib, panobinostat, pomalidomide, raloxifene, regorafenib, ribavirin, ruxolitinib, sacituzumab, selinexor, sorafenib, sunitinib, talazoparib, tamoxifen, thalidomide, tipifarnib, topotecan, toremifene, trabectedin, trametinib, tretinoin, trodelvy, vandetanib, venetoclax, vismodegib, and vorinostat.

Non-limiting examples of tandem repeat polymorphisms, chemotherapeutic drugs, and enzyme products for metabolizing the same contemplated for use in the present disclosure are further described in the sections entitled “Example repeat sequences,” above, and “Therapeutic agents,” below.

In some embodiments, the method further comprises: when the respective candidate genotype in the plurality of candidate genotypes having the highest corresponding likelihood indicates that the subject is at an actionable level of risk for adverse drug reaction, adjusting a dosage of the first chemotherapeutic drug in the subject; and when the respective candidate genotype in the plurality of candidate genotypes having the highest corresponding likelihood indicates that the subject is within a normal range of risk for adverse drug reaction, maintaining the dosage of the first chemotherapeutic drug in the subject.

In some embodiments, the method further comprises: when the respective candidate genotype in the plurality of candidate genotypes having the highest corresponding likelihood indicates that the subject is at an actionable level of risk for adverse drug reaction, prescribing to the subject a second chemotherapeutic drug other than the first chemotherapeutic drug; and when the respective candidate genotype in the plurality of candidate genotypes having the highest corresponding likelihood indicates that the subject is within a normal range of risk for adverse drug reaction, continuing an administration of the first chemotherapeutic drug in the subject.

In some embodiments, the method further comprises using the respective candidate genotype in the plurality of candidate genotypes having the highest corresponding likelihood to determine a risk of disease for the subject, where the disease is selected from the group consisting of Fragile X syndrome, amyotrophic lateral sclerosis (ALS), Huntington's disease, Friedreich's ataxia, spinocerebellar ataxia, spino-bulbar muscular atrophy, myotonic dystrophy, Machado-Joseph disease, dentatorubral pallidoluysian atrophy, benign familial hyperbilirubinemia, and Gilbert syndrome. In some embodiments, the disease is selected from the group consisting of Fragile X syndrome, Spinocerebellar ataxia 12, Spinocerebellar ataxia 1, Spinocerebellar ataxia 2, Spinocerebellar ataxia 3, Spinocerebellar ataxia 6, Spinocerebellar ataxia 7, Huntington's disease, Spinal and bulbar muscular atrophy, Dentatorubral-pallidoluysian atrophy, Friedreich's ataxia, Myotonic dystrophy 2, Spinocerebellar ataxia 10, Spinocerebellar ataxia 31, Spinocerebellar ataxia 36, Amyotrophic lateral sclerosis, multiple skeletal dysplasias, Synpolydactyly syndrome, Hand-foot-genital syndrome, Cleidocranial dysplasia, Holoprosencephaly, Oculopharyngeal muscular atrophy, Blepharophimosis, ptosis, epicanthus inversus syndrome, ARX-related X-linked mental retardation, Myotonic dystrophy 1, Spinocerebellar ataxia 8, Huntington's disease-like 2, Myoclonic epilepsy of Unverricht and Lundborg, and Spinocerebellar ataxia 17. In some embodiments, the method further comprises using the respective candidate genotype in the plurality of candidate genotypes having the highest corresponding likelihood to determine a severity of disease for the subject. For instance, in some embodiments, the method further comprises using the respective candidate genotype in the plurality of candidate genotypes having the highest corresponding likelihood to determine a risk of disease for the subject, and, when the risk of disease determines that the subject is likely to have the disease, using the respective candidate genotype in the plurality of candidate genotypes having the highest corresponding likelihood to determine a corresponding severity of the disease for the subject.

In some embodiments, the method further comprises validating the respective candidate genotype in the plurality of candidate genotypes having the highest corresponding likelihood using one or more validation procedures.

In some embodiments, the one or more validation procedures comprises obtaining a set of long-read sequence reads, wherein the set of long-read sequence reads is obtained from a long-read sequencing of a second plurality of nucleic acids in a validation sample from the subject, and using the set of long-read sequence reads to determine a validation genotype of the subject for the genomic locus comprising the tandem repeat, from the plurality of candidate genotypes. In some embodiments, the long-read sequencing is single molecule sequencing or synthetic long-read sequencing.

Alternatively or additionally, another aspect of the present disclosure provides systems and methods for obtaining a second set of sequence reads, where the second set of sequence reads do not map to the repeat sequence (e.g., the tandem repeat) in the genomic locus. In some such embodiments, the second set of sequence reads map to another genomic region other than the repeat sequence (e.g., the tandem repeat) in the genomic locus. In some such embodiments, the second set of sequence reads are unmapped.

In some embodiments, the second set of sequence reads is obtained from a sequencing of nucleic acids from the same or a different biological sample from which the first set of sequence reads was derived. In some embodiments, the second set of sequence reads is obtained from the same or a different sequencing data set (e.g., the same or a different sequencing analysis) from which the first set of sequence reads was derived.

In some embodiments, the one or more validation procedures further comprises: obtaining, in electronic form, a second set of sequence reads obtained from the biological sample of the subject that map to an auxiliary genomic region other than the tandem repeat in the genomic locus; determining, using the second set of sequence reads, a variant call for the auxiliary genomic region; and using at least (i) the respective candidate genotype in the plurality of candidate genotypes having the highest corresponding likelihood and (ii) the variant call to determine a risk of adverse drug reaction for a first chemotherapeutic drug.

In some implementations, the auxiliary genomic region comprises a nucleotide sequence for a second tandem repeat, other than the tandem repeat in the genomic locus. In some implementations, the auxiliary genomic region comprises a nucleotide sequence for a genomic variant other than a repeat sequence polymorphism. In some implementations, auxiliary genomic region is for a same or different gene than the genomic locus comprising the tandem repeat.

For instance, in an example embodiment where the genomic locus comprising the tandem repeat is the UGT1A1 gene, the auxiliary genomic region comprises a nucleotide sequence for a genomic variant other than a repeat sequence polymorphism in the UGT1A1 gene (e.g., a single nucleotide variant or an insertion deletion). In another example embodiment where the genomic locus comprising the tandem repeat is the UGT1A1 gene, the auxiliary genomic region comprises a nucleotide sequence for a genomic variant other than a repeat sequence polymorphism in a different gene other than the UGT1A1 gene (e.g., a single nucleotide variant or an insertion deletion in the DPYD gene). In yet another example embodiment where the genomic locus comprising the tandem repeat is the UGT1A1 gene, the auxiliary genomic region comprises a nucleotide sequence for a second tandem repeat in a different gene other than the UGT1A1 gene.

In some embodiments, the method further comprises using at least (i) the respective candidate genotype in the plurality of candidate genotypes having the highest corresponding likelihood and (ii) the variant call to determine a risk of disease for the subject.

In some embodiments, the variant call is a single nucleotide variant (SNV), a multi-nucleotide variant (MNV), or an insertion deletion (indel). In some embodiments, the auxiliary genomic region is the DPYD gene.

In some embodiments, the determining a variant call for the auxiliary genomic region further comprises inputting at least the second set of sequence reads into a trained deep neural network model, thereby obtaining, as output from the trained deep neural network model, the variant call for the auxiliary genomic region.

Suitable methods for using the candidate genotype in the plurality of candidate genotypes having the highest corresponding likelihood that are contemplated for use in the present disclosure, including but not limited to: using the candidate genotype in the plurality of candidate genotypes having the highest corresponding likelihood to inform clinical or research based decision-making; validating the respective candidate genotype in the plurality of candidate genotypes having the highest corresponding likelihood; obtaining variant calls; using the candidate genotype in the plurality of candidate genotypes having the highest corresponding likelihood in a pipeline for clinical or research based decision-making; and/or generating and using reports for clinical and/or research based decision-making, are further described in the section entitled “Further methods and applications,” below.

4. Assigning Likelihood of Candidate Genotypes

An illustrative example of a Bayesian variant caller will now be described.

In the example embodiment, the input to the model consists of the counts of the number of sequence reads fᵣ with r repeats (e.g., of the TA element) in reads observed spanning the genomic locus comprising the tandem repeat (e.g., the site of the variant). In other words, for each sequence read in the first set of sequence reads, a corresponding repeat count r of the number of repeat units in the tandem repeat is determined. For each value of r, the number of sequence reads fᵣ having the corresponding repeat count r can then be determined.

Letting R denote the complete set of relevant reads (e.g., the first set of sequence reads that map to the genomic locus comprising the tandem repeat), it results in:

$$\# R = {\sum }_{r} {f}_{r} .$$

In the example embodiment, Bayes' theorem is used to adjust probabilities given new evidence in the following way:

$$P ( H \vert E ) = \frac{P ( E \vert H ) P ( H )}{P ( E )} , \qquad [ Equation 2 ]$$

where:

E represents the evidence or data that has been seen in the experiment. In embodiments of the present methods and systems, E comprises the counts of the number of reads exhibiting a given number of repeats of TA (that is, the numbers fᵣ).

H represents a specific hypothesis. For example, in an embodiment, the hypothesis is “the sample is heterozygous with one allele having 6 repeats and other allele having 8 repeats.” In some such embodiments, the set of hypotheses H is defined as encompassing all the possibilities to be modeled. In some such embodiments, if there are n haplotypes (e.g., candidate alleles) and diploid samples are assumed, then #H=n(n+1)/2.

P(H) is the prior probability of H; that is, the probability of H before seeing any evidence. While in principle priors could be used based on observed population counts, instead some embodiments of the present methods and systems utilize the principle of indifference and assign all hypotheses the same prior, P(H)=1/#H. Because the total number of reads is comparatively high, in some such embodiments, the priors have only a small effect on the result. Alternatively, in some embodiments, setting an appropriate prior has a larger effect on the result (e.g., is more important) when making the call from low coverage data.

P(E|H) is the conditional probability of seeing the evidence E if the hypothesis H happens to be true. It is also called a likelihood function when it is considered as a function of H for fixed E. Generally, this quantity is of mainly theoretical significance as a way of deriving the formulae wanted for P(H|E).

P(E) is the marginal probability of E, or the prior probability of witnessing the new evidence E when it is not yet known which hypothesis is true. This term is effectively a normalization factor and it can be calculated by:

$$P ( E ) = {\sum }_{H} P ( E \vert H ) P ( H ) .$$

In practice, in some embodiments, this term cancels out and can be ignored during computation.

P(H|E) is called the posterior probability of H given E. In some embodiments, this is used to select the most likely (e.g., best) hypothesis and is ultimately the term to be computed.

To compute P(E|H), in the example embodiment, it is assumed that each read is independent, thus the overall probability is simply the product of probabilities across all reads:

$$P ( H ) = {\prod }_{R ∈ R} P ( H )$$

where P(R|H) is the probability of the observed read given the hypothesis.

In some embodiments, the pertinent information in a respective sequence read is taken to be the corresponding repeat count of the number of repeat units in the tandem repeat. In some such embodiments, given that the only pertinent information in the read is number of TA repeats r, the product can be rewritten in terms of the counts fᵣ to give:

$$P ( E \vert H ) = {\prod }_{r} {P ( r \vert H )}^{{f}_{r}} , \qquad [ Equation 3 ]$$

where now P(r|H) is probability of observing r repeats in a read with the hypothesis. In some embodiments, all fᵣ reads with r repeats are treated as a single term in the product. In some such embodiments, although all fᵣ reads with r repeats are treated as a single term in the product, it is still assumed that each of these reads is independent. In particular, in some embodiments, PCR duplicates are removed from the set of reads before computing fᵣ.

Because the total number of reads can be high (e.g., hundreds or more), in some implementations, care is needed in the calculation to avoid arithmetic underflow. Therefore, in some such embodiments, the calculation is done in logarithmic space:

$$log P ( E \vert H ) = {\sum }_{r} f ( r ) log P ( r \vert H ) .$$

In some implementations, the computation of P(r|H) depends on both r and H and on whether H is homozygous or heterozygous. If p(r|h) denotes the probability of observing r repeats when a haplotype (e.g., candidate allele) has h repeats and if H is the diploid hypothesis {a, b} (e.g., one allele has a repeats and the other has b repeats), then:

$$P ( r \vert H ) = \{ \begin{matrix}\frac{1}{2} [ p ( r \vert a ) + p ( r \vert b ) ] & if a \neq b ( heterozygous ) \\ p ( r \vert a ) & if a = b ( homozygous )\end{matrix}$$

In some embodiments, the model probabilities used for p(r|h) are obtained from empirical distributions found by analysis of one or more reference samples. In some embodiments, the reference samples are solid tumor and/or normal tissue samples. In some embodiments, the analysis is obtained using hybrid capture next-generation sequencing for a targeted panel of genes against the reference samples (e.g., a list of solid tumor and hematologic malignancy target genes in a targeted oncology panel). In some embodiments, the analysis is a tumor-normal matched oncology NGS sequencing assay. In some embodiments, the analysis is a targeted panel NGS sequencing assay.

In some embodiments, the model probabilities provide a plurality of repeat count adjustment factors that can be used to obtain the corresponding first likelihood, for each respective candidate allele in the plurality of candidate alleles, that the respective candidate allele is present in the sample of the subject (e.g., via the calculation of P(E|H)). Illustrative repeat count adjustment factors for a plurality of candidate alleles (“h”) and for a numerical range of repeat units (“r”) are shown below in Table 3.

TABLE 3
Exemplary Model Probabilities

| r | h = 6 | h = 7 | h = 8 | h = 9 |
|---|---|---|---|---|
| 2 | 0.0000000000 | 0.00000e000 | 0.0000000000 | 0.0000000 |
| 3 | 0.0000000000 | 1.771968e−06 | 0.0000000000 | 0.0000000 |
| 4 | 0.0000000000 | 5.969332e−05 | 5.864456e−06 | 0.0000000 |
| 5 | 0.0335307143 | 4.136760e−03 | 6.836893e−04 | 0.0004595 |
| 6 | 0.9450305714 | 5.395183e−02 | 1.194826e−02 | 0.0018380 |
| 7 | 0.0212502857 | 8.874152e−01 | 8.029274e−02 | 0.0198720 |
| 8 | 0.0001885714 | 5.246976e−02 | 8.207345e−01 | 0.0887150 |
| 9 | 0.0000000000 | 3.716483e−03 | 8.103252e−02 | 0.8154375 |
| 10 | 0.0000000000 | 3.204899e−04 | 8.015238e−03 | 0.0631995 |
| 11 | 0.0000000000 | 4.529438e−05 | 9.953915e−04 | 0.0095590 |
| 12 | 0.0000000000 | 1.104789e−05 | 1.793756e−04 | 0.0009190 |

Without being limited to any one theory of operation, in some embodiments, a distribution of probabilities used for p(r|h) is expected to peak for values at p(r|r). Similarly, it is noted that the distributions in Table 3 are sharply peaked at p(r|r).

In some embodiments, the methods and systems of the present disclosure include obtaining a plurality of repeat count adjustment factors using an error model, where the plurality of repeat count adjustment factors can be used to obtain the corresponding first likelihood, for each respective candidate allele in the plurality of candidate alleles, that the respective candidate allele is present in the sample of the subject (e.g., via the calculation of P(E|H)). In some implementations, the error model is a simple error model.

Adjustment factors, and methods of obtaining the same, that are contemplated for use in the present disclosure are further described below (see, e.g., the section entitled “Adjustment factors”).

In some embodiments, each respective adjustment factor is a non-zero number. For instance, in some such embodiments, each respective value for p(r|h) is a non-zero number. Without being limited to any one theory of operation, in some such embodiments, unpredictable and/or severe consequences (e.g., black swan events) can be avoided.

In some implementations, each respective adjustment factor (e.g., p(r|h)) is adjusted to be a non-zero number by applying a non-zero constant to the respective adjustment factor. In some such implementations, the plurality of repeat count adjustment factors (e.g., the distribution of p(r|h)) is normalized to account for the addition of the non-zero constant to each respective adjustment factor in the plurality of adjustment factors. In some embodiments, the non-zero constant is ∈=10⁻⁹. In some embodiments, the non-zero constant ∈ is no more than 0.1, no more than 0.01, no more than 0.001, no more than 10⁻⁴, no more than 10⁻⁵, no more than 10⁻⁶, no more than 10⁻⁷, no more than 10⁻⁸, no more than 10⁻⁹, or no more than 10⁻¹⁰. In some embodiments, the non-zero constant ∈ is at least 10⁻¹¹, at least 10⁻¹⁰, at least 10⁻⁹, at least 10⁻⁸, at least 10⁻⁷, at least 10⁻⁶, at least 10⁻⁵, at least 10⁻⁴, at least 0.001, or at least 0.01.

Returning again to the illustrative example of the Bayesian variant caller, in some embodiments, the method comprises computing the posterior probability of each hypothesis H given the observed evidence E, or P(H|E), for each respective hypothesis in the set of hypotheses (e.g., each H∈H).

For instance, referring to FIGS. 7 and 8, in some embodiments, for each respective haplotype (e.g., candidate allele) having a respective repeat number h (e.g., 5, 6, 7, 8, 9, or 10), the variant caller provides a posterior probability that the sample of the subject has the respective haplotype, based at least on the proportion of sequence reads in the plurality of sequence reads that have the repeat count of the number of repeat units corresponding to the respective candidate allele (e.g., “repeat spanning read count”) and (ii) a repeat count adjustment factor matching the number of repeat units corresponding to the respective candidate allele from the set of repeat count adjustment factors for the respective candidate allele. See, for example, Equations 2 and 3, above.

In some such embodiments, for each respective candidate allele in the plurality of candidate alleles, the respective posterior probability is the corresponding first likelihood that the sample of the subject has at least the respective candidate allele.

In some embodiments, the methods and systems provided herein comprise evaluating the subject (a) under a consideration of homozygosity at the genomic locus by selecting the respective candidate allele in the plurality of candidate alleles having the highest corresponding first likelihood, or (b) under a consideration of heterozygosity at the genomic locus by selecting a pair of candidate alleles in the plurality of candidate alleles respectively having the highest and second highest corresponding first likelihood.

Accordingly, as illustrated in FIG. 7, a consideration of homozygosity at the genomic locus results in selection of the highest corresponding first likelihood of a candidate allele (e.g., a posterior probability of 0.9 for the candidate allele in a homozygous genotype having a repeat sequence of (TA)₈ for the UGT1A1 promoter region). The determined genotype for the genomic locus is thus (TA)₈/(TA)₈ (“Final call”). Alternatively, as illustrated in FIG. 8, a consideration of heterozygosity at the genomic locus results in selection of the highest two first likelihoods for corresponding candidate alleles (e.g., posterior probabilities of 0.75 and 0.7 for candidate alleles in a heterozygous genotype having repeat sequences of (TA)₇ and (TA)₈ for the UGT1A1 promoter region, respectively). The determined genotype for the genomic locus is thus (TA)₇/(TA)₈ (“Final call”).

Returning again to the illustrative example of the Bayesian variant caller, after computing P(H|E) for each H∈H, in some embodiments, one or more quality metrics are determined. For instance, in some such embodiments, after a maximum likelihood is determined for one or a pair of candidate alleles, a log-odds ratio of the corresponding hypothesis (e.g., the selected genotype) is reported as a quality metric according to the following:

$$log \frac{P ( H \vert E )}{1 - P ( H \vert E )} = log \frac{P ( H \vert E )}{{\sum }_{J ∈ H , J \neq H} P ( J \vert E )}$$

In some embodiments, the quality metric is reported as a genotype quality (GQ). In some embodiments, the quality metric is Phred-scaled (e.g., a Phred-scaled GQ score). See, for instance, GATK Team, “Phred-scaled quality scores,” Broad Institute, available on the Internet at gatk.broadinstitute.org/hc/en-us/articles/360035531872-Phred-scaled-quality-scores. Phred-scaled genotype quality scores are illustrated, for example, in FIGS. 7 and 8 (“Final call: GQ”). In some embodiments, one or more maximum likelihoods, determined genotypes, and/or quality metrics are outputted in Variant Call Format (VCF). Other quality metrics are contemplated for use in the present disclosure, as will be apparent to one skilled in the art. For instance, in some implementations, the methods and systems disclosed herein comprise determining a QUAL score and/or another non-identity posterior probability representing the probability that the sample is different to the homozygous reference.

In some embodiments, one or more determined genotypes (e.g., genotype calls) are filtered based on the one or more quality metrics. In some such embodiments, the filtering removes one or more determined genotypes of poor quality, such as those resulting from low-quality specimens and/or other experimental errors.

In some embodiments, the filtering is performed using a threshold quality metric score. In some embodiments, a respective candidate allele is removed from the plurality of candidate alleles when a corresponding quality metric obtained for the respective candidate allele fails to satisfy the threshold quality metric score. In some implementations, a respective genotype call is removed from a set of genotype calls when a corresponding quality metric obtained for the respective genotype call fails to satisfy the threshold quality metric score.

In some embodiments, the quality metric is a genotype quality (GQ), and the threshold quality metric score is at least 50, at least 60, at least 70, at least 75, at least 80, at least 85, at least 90, or at least 95. For instance, in some embodiments, the threshold quality metric score is a GQ score of 70.

5. Adjustment Factors

As described above, in some embodiments, the obtaining a plurality of sets of repeat count adjustment factors is performed using empirical distributions found by analysis of sequence reads from one or more reference samples. For instance, in some such embodiments, the analysis is a tumor-normal matched oncology NGS sequencing assay. Alternatively or additionally, in some embodiments, the analysis is a targeted panel NGS sequencing assay. In some implementations, the plurality of sets of repeat count adjustment factors is manually determined based on one or more sequence read distributions (e.g., read counts, repeat count probabilities).

In some implementations, each respective repeat count adjustment factor is a respective probability that a sequence read will have a respective number of repeat units in a numerical range of repeat units, given that the originating sample has a haplotype with a corresponding number of repeat units corresponding to the respective candidate allele.

In other words, in some such implementations, for each respective candidate allele having a corresponding number of repeat units h and for each respective number of repeat units r in the numerical range of repeat units, the corresponding repeat count adjustment factor is a respective probability of observing a sequence read having r repeats given a sample having a haplotype with h repeats.

In some embodiments, as described above, the obtaining a plurality of repeat count adjustment factors comprises using an error model. In some embodiments, the error model is generated using a prior distribution of sequence reads (e.g., a distribution of sequence read counts and/or repeat count probabilities). For instance, in some implementations, the error model is fitted to an empirically derived distribution of probabilities that a sequence read will have a respective number of repeat units in a numerical range of repeat units, for each repeat length in a plurality of possible candidate allele repeat lengths. In some such implementations, the empirically derived distribution is determined using a tumor-normal matched oncology NGS sequencing assay. In some implementations, the empirically derived distribution is determined using a targeted panel NGS sequencing assay.

An illustrative example of a simple error model for obtaining repeat count adjustment factors will now be described.

In some embodiments, a simple error model (e.g., simple stutter model) provides distribution p(r|h) (and later p′(r|h)) that represents the probability of observing r repeats in a haplotype (e.g., candidate allele) with h repeats. Without being limited to any one theory of operation, in some implementations, this aspect of the variant caller accounts for errors in the reads that are caused by the stutter introduced by DNA polymerase in the PCR amplification step of the NGS sequencing.

To account for this issue, in some implementations, a probability s is assumed for any particular repeat unit (e.g., TA pair) being in error. In some implementations, deletion of a respective repeat unit (e.g., TA pair) and insertion of an extra copy are further assumed to be equally likely; that is, having a probability of s/2. In some embodiments, the probability p(r|h) of observing r repeats when the haplotype has h repeats is then computed according to the following recurrence:

$$p ( 0 \vert 0 ) = 1 , p ( r \vert h ) = ( 1 - s ) p ( r - 1 \vert h - 1 ) + \frac{s}{2} p ( r - 2 \vert h - 1 ) + \frac{s}{2} p ( r \vert h - 1 ) , \qquad [ Equation 1 ]$$

for 0≤r≤2h and 0 elsewhere. Note,

$${\sum }_{r = 0}^{2 h} p ( r \vert h ) = 1 .$$

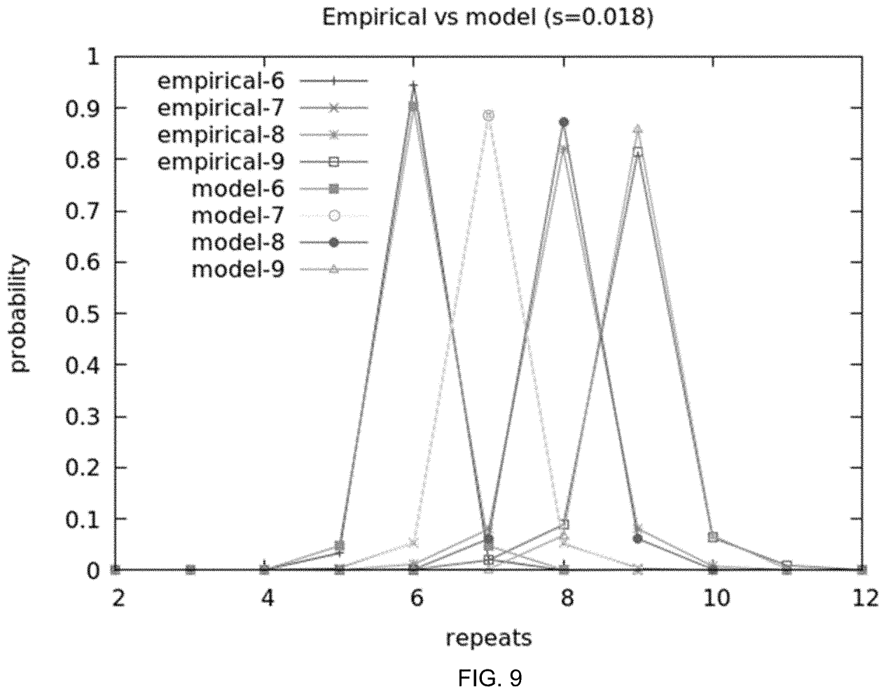

FIG. 9 illustrates an example embodiment in which s=0.0173, where s is selected to fit r=7 using data from an empirically derived distribution of probabilities. Comparison of predicted probabilities generated by the error model against the empirically derived probabilities shows that the empirically observed distribution is well approximated by the simple error model. Close approximation was observed for candidate alleles having repeat counts of 6, 7, 8, and 9 repeat units, for all comparisons between the empirically observed distribution (“empirical”) and the error model (“model”).

Advantageously, in some embodiments, the error model is used to generate repeat count adjustment factors (e.g., model probabilities) for numbers of repeat units (e.g., values of r and/or h) that are not represented in the prior distribution (e.g., the empirically derived distribution) to which the error model is fitted. For example, in some such embodiments, the error model is used to generalize to arbitrary repeat lengths for which empirical data is not available. In some embodiments, the error model is used to construct prior probabilities for determining posterior probabilities, in accordance with the methods and systems disclosed herein (see, e.g., the section entitled “Assigning likelihood of candidate genotypes,” above).

Returning again to the illustrative example, in some such embodiments, the simple stutter model is only non-zero for 0≤m≤2h. Such embodiments occur, at least in part, because each repeat unit (e.g., TA pair) can only give rise to at most one additional copy. In some cases, one or more candidate alleles comprise even greater numbers of repeats than 2h. To account for this possibility, in some implementations, the probability previously assigned to p(2h|h) is spread across all longer repeats according to:

$${{p}^{′}}^{( r \vert h )} = \{ \begin{matrix}p ( r \vert h ) & if r < 2 h \\ \frac{p ( 2 h \vert h )}{{2}^{r - 2 h + 1}} & if r \geq 2 h\end{matrix} .$$

In such embodiments, p′(r|h) is a strictly positive probability distribution (p′(r|h)>0), such that:

$${\sum }_{r = 0}^{∞} {p}^{′} ( r \vert h ) = 1 .$$

In this way, in some embodiments, the methods and systems disclosed herein facilitate genotyping of repeat sequences by providing a caller that identifies the highest probability genotype for the sequence reads present in a sample from a subject. In some such embodiments, the determination of genotypes is performed using a Bayesian variant caller. In some implementations, the methods and systems disclosed herein further facilitate genotyping of repeat sequences by, optionally, removing erroneous reads present in the sequences because of DNA polymerase stutter.

6. Further Methods and Applications

In some embodiments, the genotype of the subject for the genomic locus is provided in a report (e.g., to a patient, clinician, researcher, and/or medical practitioner).

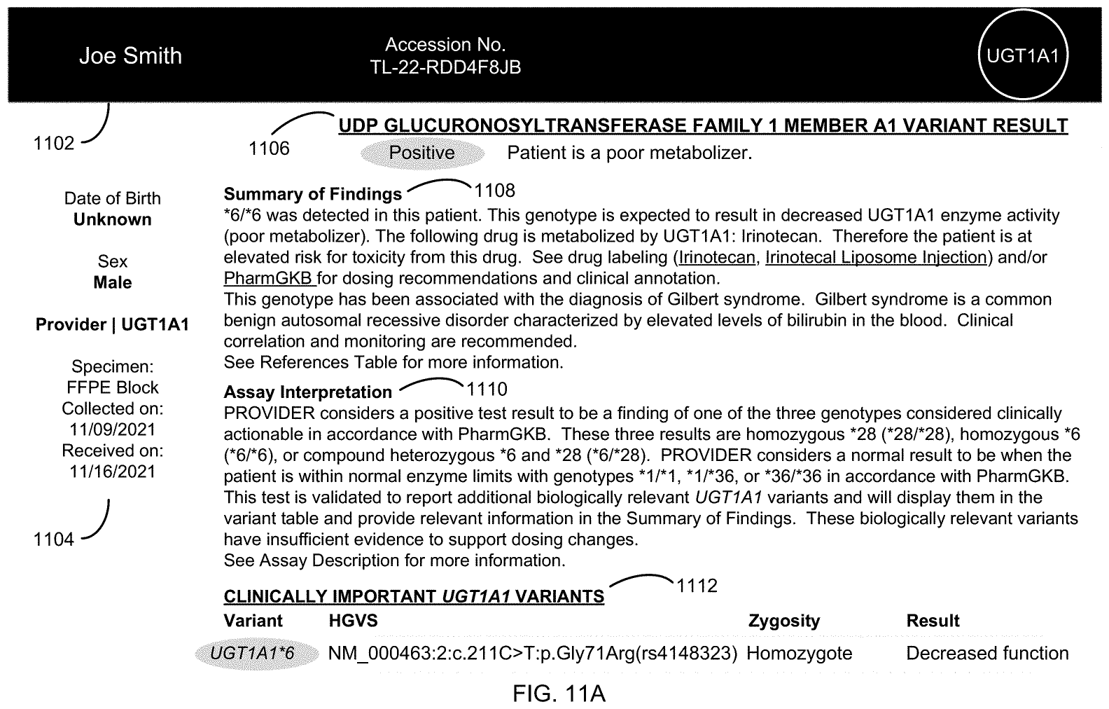

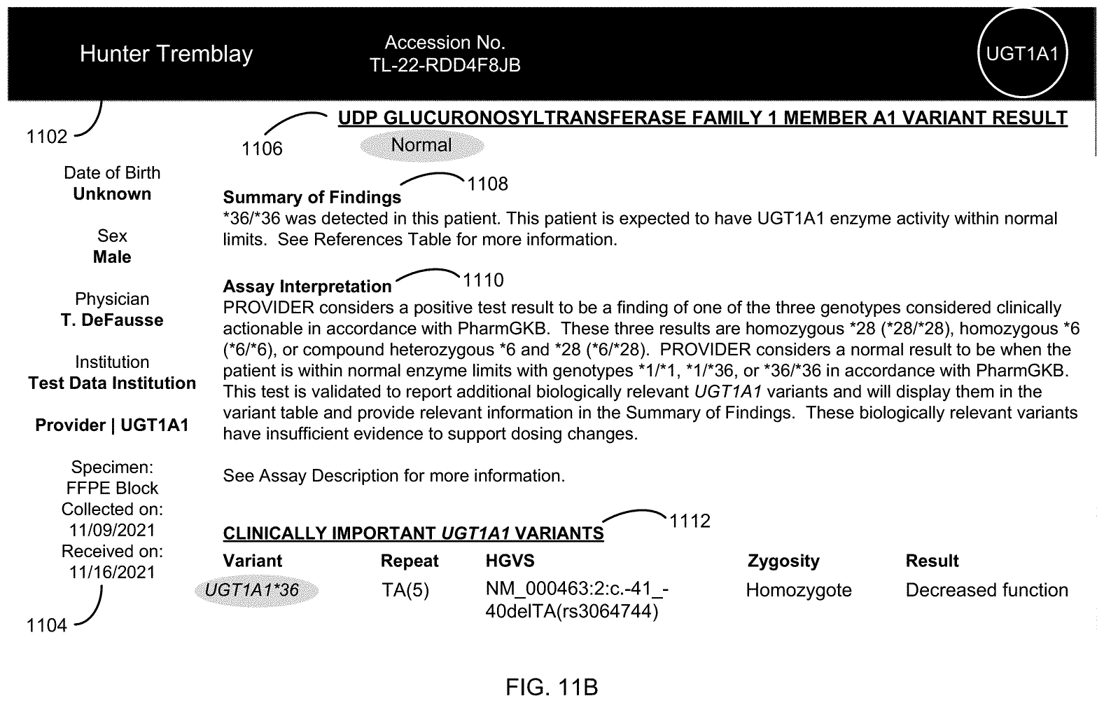

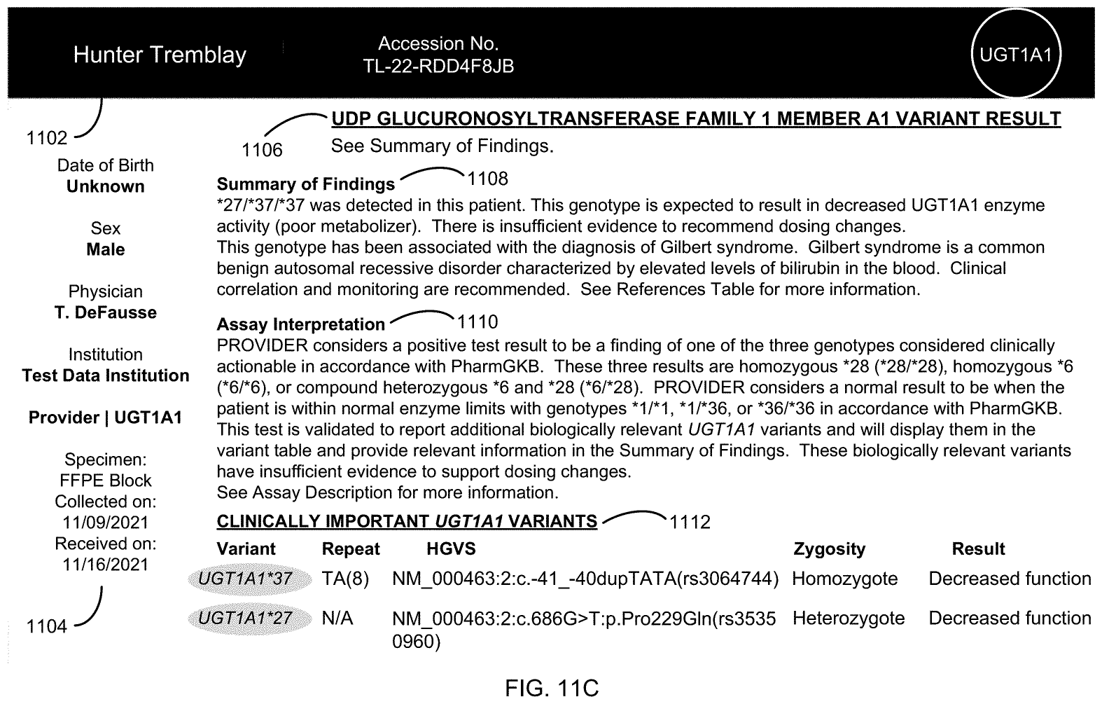

Some embodiments of the present methods also involve building of a genotype profile for one or more determined genotypes, specifically, repeat sequence polymorphisms, for a particular patient sample. As illustrated in FIGS. 11A-C, in some embodiments, a repeat genotype profile is a particular example of a report that can be provided, in accordance with the methods and systems of the present disclosure. In some implementations, the data that populates the repeat genotype report is obtained from the methods provided herein. In some embodiments, a repeat sequence polymorphism identifier is utilized across multiple patient reports where the same repeat sequence polymorphism is found, providing consistent identification and association of that repeat sequence polymorphism with future measurements as they occur with different patients, such as therapeutic outcomes. This is particularly useful, for instance, when the present method is used for the initial identification and documentation of a novel repeat sequence polymorphism.

In some implementations, repeat sequence polymorphism reports are of use for clinical and/or research based decision-making. While adaption of the precise contents of the report section is anticipated to be part of the repeat sequence polymorphism determination method, such adaption is believed to be well within the purview of one of ordinary skill, once the identification of the repeat sequence polymorphisms involved is obtained. However, it should be emphasized that certain embodiments of the present method involving repeat sequence polymorphisms include the association newly discovered polymorphisms with patient data, such as therapeutic response, therapeutic non-response, and overall clinical outcome. Advantageously, in some implementations, the repeat sequence polymorphism reports, when supported by multiple patient samples showing presence or absence of the same repeat sequence polymorphisms, provide valuable input into clinical decision-making for diseases or drug treatment side effects associated with such. In some implementations, a repeat sequence polymorphism report is used to provide a clinical or research based recommendation, such as a recommendation for clinical correlation and/or monitoring of a patient.

In some embodiments, the repeat sequence polymorphism report is used to provide a quantitative basis for decisions such as providing data surrounding polymorphisms that can be targeted by a therapy or drug; polymorphisms that are biomarkers for successful response or a particular variation in administered amount of a therapy or drug; polymorphisms known to affect disease course or prognosis; and/or polymorphisms that can help with diagnosis. Additionally, in some embodiments, the repeat sequence polymorphisms are used to provide quantitative basis for decisions involved in research-based decision-making such as polymorphisms that can be targeted by a therapy or drug; polymorphisms that are biomarkers for successful response or a particular variation in administered amount of a therapy or drug; polymorphisms known to affect disease course or prognosis; and/or polymorphisms that can help with diagnosis. Alternatively or additionally, in some implementations (e.g., at the research level), a repeat sequence polymorphism report provides an overview of polymorphisms in the patient or specimen, for example, addressing whether there are a polymorphisms in patients suffering from a particular disease as compared to a typical specimen. In some such implementations, the repeat sequence polymorphism report indicates the presence or absence of repeat sequence polymorphisms in a sample generally.

In some embodiments, the methods and systems disclosed herein further comprise developing a companion diagnostic test for a treatment method of a disease based on the presence or absence of one or more repeat sequence polymorphisms in a patient sample. In some embodiments, the companion diagnostic test is developed using a report, such as a repeat sequence polymorphism report, generated using the methods and systems disclosed herein.

In some implementations, the development of companion diagnostic tests considers at least two factors. First, as discussed above, there are a wide range of diseases associated with repeat sequence polymorphisms, and as this is an active area of research, more and more diseases are being linked to such associations. There is also the abovementioned association of higher probability of adverse events with particular drug treatments in the presence of certain repeat sequence polymorphisms. Such biological impact of alternative repeat sequences provides strong motivation for the production of repeat sequence polymorphism reports for individual or groups of patient samples. Companion diagnostics are defined by the FDA as a device that “provides information that is essential for the safe and effective use of a corresponding drug or biological product,” and such companion diagnostics aim to help health care professionals determine whether the benefits of a specific therapy outweigh potential side effects or risks (see, Nalley, Oncology Times, 39(9):24-26, discussing the use of companion diagnostics in the oncology setting). Thus, in certain embodiments, the methods and systems disclosed herein are used to provide information that can be associated with the safe and effective use of a corresponding drug.

In some embodiments, the methods and systems disclosed herein further comprise one or more steps selected from the group consisting of: preparing reports (e.g., repeat sequence polymorphism reports) for one or more patients in a plurality of patients suffering from a disease; associating the treatment response of the one or more patients to a particular treatment method for the disease; determining a further association between positive treatment responses and the presence or absence of one or more particular repeat sequence polymorphisms in patient samples; and/or using the presence or absence of the particular repeat sequence polymorphism to identify additional patients more likely to benefit from the treatment method than those patients without the presence or absence of the particular repeat sequence polymorphisms in their report, thus providing a companion diagnostic for the particular treatment method for the disease. In an example embodiment, one use of this method is when the disease is cancer and the treatment method is one which is known to be impacted by varying expression of enzymes involved in the metabolism of the chemotherapy drug administered.

Examples of cancer include, but are not limited to, carcinoma, lymphoma, blastoma, glioblastoma, sarcoma, and leukemia. In some embodiments, non-limiting cancers include breast cancer, squamous cell cancer, lung cancer (including small-cell lung cancer, non-small cell lung cancer (NSCLC), adenocarcinoma of the lung, and squamous carcinoma of the lung (e.g., squamous NSCLC)), various types of head and neck cancer (e.g., HNSC), cancer of the peritoneum, hepatocellular cancer, gastric or stomach cancer (including gastrointestinal cancer), pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, and hepatic carcinoma, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), hairy cell leukemia, chronic myeloblastic leukemia, and post-transplant lymphoproliferative disorder (PTLD), as well as abnormal vascular proliferation associated with phakomatoses, edema (such as that associated with brain tumors), and/or Meigs' syndrome.

As would be well understood by one of ordinary skill, the term “cancer” for use with methods and systems of the present disclosure is not limited only to primary forms of cancer, but also involves cancer subtypes. Some such cancer subtypes are listed above but also include breast cancer subtypes such as Luminal A (hormone receptor (HR)+/human epidermal growth factor receptor (HER2)−); Luminal B (HR+/HER2+); Triple-negative or (HR−/HER2−) and HER2 positive. Other cancer subtypes include the various lung cancers listed above and prostate cancer subtypes involving changes in E26 transformation specific genes (ETS; specifically ERG, ETV1/4, and FLI1 genes) and subsets defined by mutations in FOXA1, SPOP, and IDH1 genes.

In some embodiments of the present disclosure, a computational format used for matching between a genotype (e.g., repeat polymorphism results) in a sample of a subject, a disease at issue, and/or any potential treatment methods is in the form of a manually curated knowledge database. In some embodiments, such a database records the particular genotype (e.g., repeat sequence polymorphism), including the gene involved with the disease state, applicable therapies, and/or the outcome of such therapies. In some implementations, each newly identified genotype (e.g., repeat sequence polymorphism) is recorded into this database as one or more local events. Because the local nature of the events makes it difficult to compare specific genotypes within the context of the entirety of a reference sequence, in some implementations, the knowledge database is manually curated. In some such implementations, this curated database provides a basis for future assignment of similar genotypes (e.g., repeat sequence polymorphisms) to the possible recommendation of therapies, particularly those where there have been positive outcomes.

In some embodiments, the methods and systems disclosed herein are used for curating or obtaining a curated database that includes patient genotypes and therapeutic outcomes, e.g., which is useful for identifying associations between particular patient genotypes in a population and therapeutic outcomes. For example, for identifying a genotype that is associated with a positive outcome when a patient is treated with a particular therapy and/or a genotype that is associated with a negative outcome when a patient is treated with a particular therapy. In some embodiments, artificial intelligence is used to curate and/or analyze the database. Databases that associate particular patient outcomes and other patient characteristics such as gene expression values to particular therapies and their outcome are known in the art. See, for example, U.S. Pat. No. 10,600,503 (Systems medicine platform for personalized oncology); U.S. Patent Publ. No. 20060136143 (Personalized genetic-based analysis of medical conditions); and U.S. Patent Publ. No. 20080082522 (Computational systems for biomedical data), each of which is expressly incorporated herein in their entireties for all purposes. In some embodiments, the knowledge database is generated using manual curation, artificial intelligence-driven curation, or a combination thereof.

In some embodiments, the methods and systems disclosed herein are utilized in research settings to elucidate the heterogeneity of therapeutic responses within and among patients, or in the clinical laboratory to potentially guide precision oncology treatments. For instance, in some embodiments, the approach is used to determine previously unknown associations between genotypes and therapeutic responses. In some example embodiments, the approach includes accessing a database storing information about, for each respective subject in a plurality of subjects, a corresponding genotype for the subject at one or more genomic loci (e.g., a genomic locus having one or more tandem repeats), a corresponding treatment administered to the respective subject for treatment of a clinical condition, and a corresponding outcome for the treatment of the subject. The method then includes determining an association between one or more treatments administered to subjects with a particular genotype and the clinical outcomes for the subjects based on the data in the database. Methods for identifying associations between variables, e.g., between genotypes and treatment outcomes, are known in the art. For example, various methods for determining associations using statistical tests, e.g., identifying statistical significance, are known in the art. Similarly, machine learning processes, such as association rule learning, can be used to identify such associations between genotype and therapeutic efficacy.

In some embodiments, a genotype of the subject at a corresponding genomic locus is obtained using any of the methods disclosed herein, and the obtaining an association is performed by associating the respective genotype of the subject with one or more therapeutic responses for the subject. In some embodiments, a plurality of genotypes for the subject at a corresponding plurality of genomic loci are determined using any of the methods disclosed herein, and the obtaining an association is performed by associating each respective genotype for the subject with one or more therapeutic responses associated with the subject.

In some embodiments, such identified associations between genotype and treatment efficacy can be used to support clinical decision making for test subjects, e.g., by determining that a test subject has a probability of responding well or poorly to a particular treatment for a clinical condition, such as cancer. For instance, in some embodiments, an identified association is used to determine whether a subject is at risk for an adverse drug reaction in response to treatment with the therapeutic agent. In some embodiments, the risk is reported as an indication of high risk, moderate risk, or low risk (e.g., within normal limits). In some embodiments, the risk is reported as a likelihood or probability that the subject will experience an adverse event. In some embodiments, an identified association is used to determine whether a subject is a poor metabolizer of the therapeutic agent. In some embodiments, the methods described herein include using an association to determine whether a subject has a resistance to a therapeutic agent for treating a clinical condition, e.g., cancer. For instance, in some embodiments, an association is used to determine whether the therapeutic agent is likely to have low efficacy in treating the disease. In some embodiments, an association is used to determine whether the subject is a high metabolizer of the therapeutic agent.

In some embodiments, the methods described herein include providing a respective recommendation for a therapy, in a plurality of recommendations, for treating the disease in the subject based on the results of the evaluation. In some embodiments, the methods described herein include administering the recommended therapy for treating the disease to the subject. In some embodiments, the recommendation for a therapy is a selection of one or more therapeutic agents in a plurality of therapeutic agents. In some embodiments, the recommendation for a therapy is a change from a first therapeutic agent to a second therapeutic agent other than the first therapeutic agent. In some embodiments, the recommendation for a therapy is a change in dosage for one or more therapeutic agents. In some embodiments, the recommendation for a therapy is a cessation of treatment by a therapeutic agent.

In some embodiments, the disclosure provides methods and systems for determining the eligibility of a subject (e.g., a cancer patient) for a clinical trial (e.g., for a candidate cancer pharmaceutical agent). In some embodiments, the methods include determining whether the cancer patient is eligible for the clinical trial based on at least a genotype of a genomic locus containing a repeat sequence.

In some embodiments, the disease is cancer, including any of the cancers disclosed above. In some embodiments, a therapy and/or therapeutic agent is any of the therapeutic agents disclosed herein (see, e.g., the section entitled “Therapeutic agents,” below).

In some embodiments, the methods and systems disclosed herein are incorporated into a pipeline for clinical and/or research-based decision-making. In some such embodiments, the determined genotypes (e.g., repeat sequence polymorphisms) are evaluated in combination with one or more additional biomarkers to perform any of the additional clinical and/or research-based decision-making steps disclosed above.

In some implementations, the one or more additional biomarkers includes a single-nucleotide variant (e.g., SNV) or a small insertion/deletion (e.g., indel) variants. In some implementations, the genomic locus comprising the tandem repeat is all or a portion of a first gene, and the one or more additional biomarkers is a corresponding variant in a second gene that is different from the first gene. In some such implementations, the corresponding variant is a repeat sequence polymorphism, an SNV, and/or an indel. In some embodiments, the first gene is UGT1A1 and the second gene is DPYD.

In some embodiments, any one or more of the further methods and applications disclosed herein are performed based on the evaluation of any number or combination of suitable biomarkers, as will be apparent to one skilled in the art. For instance, in some such embodiments, the pipeline is used for one or more of preparing reports; developing companion diagnostic tests; associating treatment responses to particular treatment methods for disease; determining associations between positive treatment responses and the presence or absence of repeat sequence polymorphisms; identifying patients likely to benefit from particular treatment methods; diagnosing sensitivity or resistance to therapeutic agents; recommending treatments for disease; administering treatment for disease; and/or selecting patients for clinical trials.

In some embodiments, the disclosure provides methods and systems for providing a report for a subject (e.g., to a subject, clinician, researcher, and/or medical practitioner). In some such embodiments, the report includes any of the information disclosed herein. For instance, in some embodiments, the report includes information relating to companion diagnostic tests; associations of treatment responses to particular treatment methods for disease; associations between positive treatment responses and the presence or absence of repeat sequence polymorphisms; predicted responses of the subject to particular treatment methods; sensitivity or resistance to therapeutic agents; recommended treatments; treatment administration status; clinical trials; or a combination thereof.

7. Subjects and Samples

In certain embodiments, samples can be obtained from sources, including, but not limited to, samples from different individuals, samples from different developmental stages of the same or different individuals, samples from different diseased individuals (e.g., individuals suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative but non-limiting embodiment, a sample is a maternal sample that is obtained from a pregnant female, for example a pregnant woman. In this instance, the sample can be analyzed using the methods described herein to provide a prenatal diagnosis of potential chromosomal abnormalities in the fetus. The maternal sample can be a tissue sample, a biological fluid sample, or a cell sample.

In certain embodiments, samples are obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

Accordingly, in certain embodiments, a sample includes or consists essentially of a purified or isolated polynucleotide, or it can include samples such as a tissue sample, a biological fluid sample, a cell sample, and the like. In other embodiments, a sample is a swab or smear, a biopsy specimen, or a cell culture. In another embodiment, a sample is a mixture of two or more biological samples, e.g., a biological sample can include two or more of a biological fluid sample, a tissue sample, and a cell culture sample.

In some embodiments, a sample (e.g., a biological sample) collected from a subject is a solid tissue sample, e.g., a solid tumor sample or a solid normal tissue sample. Methods for obtaining solid tissue samples, e.g., of cancerous and/or normal tissue are known in the art, and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a sample collected from a subject is a liquid biological sample, also referred to as a liquid biopsy sample. In some embodiments, one or more samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, blood samples are collected from patients in commercial blood collection containers, e.g., using a PAXgene® Blood DNA Tubes. In some embodiments, saliva samples are collected from patients in commercial saliva collection containers, e.g., using an Oragene® DNA Saliva Kit.

Liquid biopsy samples include cell free nucleic acids, including cell-free DNA (cfDNA). As described above, cfDNA isolated from cancer patients includes DNA originating from cancerous cells, also referred to as circulating tumor DNA (ctDNA), cfDNA originating from germline (e.g., healthy or non-cancerous) cells, and cfDNA originating from hematopoietic cells (e.g., white blood cells). The relative proportions of cancerous and non-cancerous cfDNA present in a liquid biopsy sample varies depending on the characteristics (e.g., the type, stage, lineage, genomic profile, etc.) of the patient's cancer.

cfDNA is a particularly useful source of biological data for various implementations of the methods and systems described herein, because it is readily obtained from various body fluids. Advantageously, use of bodily fluids facilitates serial monitoring because of the ease of collection, as these fluids are collectable by non-invasive or minimally-invasive methodologies. This is in contrast to methods that rely upon solid tissue samples, such as biopsies, which often times require invasive surgical procedures. Further, because bodily fluids, such as blood, circulate throughout the body, the cfDNA population represents a sampling of many different tissue types from many different locations.

In some embodiments, a liquid biopsy sample is separated into two different samples. For example in some embodiments, a blood sample is separated into a blood plasma sample, containing cfDNA, and a buffy coat preparation, containing white blood cells. As used herein, the terms “blood,” “plasma,” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In some embodiments, a dedicated normal sample is also collected from a subject, for co-processing with a solid or liquid cancer sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, e.g., a swab, in the subjects mouth and rubbing it against their cheek, e.g., for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

The samples collected from the patient are, optionally, sent to various analytical environments (e.g., sequencing lab 230, pathology lab 240, and/or molecular biology lab 250) for processing (e.g., data collection) and/or analysis (e.g., feature extraction). For instance, in some embodiments, wet lab processing includes cataloguing samples (e.g., accessioning), examining clinical features of one or more samples (e.g., pathology review), and nucleic acid sequence analysis (e.g., extraction, library prep, capture+hybridize, pooling, and sequencing). In some embodiments, the workflow includes clinical analysis of one or more samples collected from the subject, e.g., at a pathology lab 240 and/or a molecular and cellular biology lab 250, to generate clinical features such as pathology features, imaging data, and/or tissue culture or organoid data.

8. Nucleic Acid Extraction from Biological Sample

In various embodiments, the nucleic acids (e.g., DNA or RNA) present in the sample are enriched specifically or non-specifically prior to use (e.g., prior to preparing a sequencing library). For instance, non-specific enrichment of sample DNA refers to the whole genome amplification of the genomic DNA fragments of the sample that can be used to increase the level of the sample DNA prior to preparing a cfDNA sequencing library. Methods for whole genome amplification are known in the art, including but not limited to degenerate oligonucleotide-primed PCR (DOP), primer extension PCR technique (PEP) and/or multiple displacement amplification (MDA).

In some embodiments, enrichment is achieved by hybridizing target nucleic acids in the sequencing library to a set of probes that hybridize to the target sequences, and then isolating the captured nucleic acids away from off-target nucleic acids that are not bound by the capture probes. In some such embodiments, target nucleic acids include nucleic acids encompassing loci that are informative for precision oncology. In some embodiments, the probe set includes probes targeting one or more gene loci, e.g., exon or intron loci. In some embodiments, the probe set includes probes targeting one or more loci not encoding a protein, e.g., regulatory loci, miRNA loci, and other non-coding loci, e.g., that have been found to be associated with a disease (e.g., cancer). In some embodiments, the plurality of loci include at least 25, 50, 100, 150, 200, 250, 300, 350, 400, 500, 750, 1000, 2500, 5000, or more human genomic loci. In some embodiments, the gene panel is a whole-exome panel that analyzes the exomes of a biological sample. In some embodiments, the gene panel is a whole-genome panel that analyzes the genome of a specimen.

In some embodiments, e.g., where a whole genome sequencing method will be used, nucleic acid sequencing libraries are not target-enriched prior to sequencing, in order to obtain sequencing data on substantially all of the competent nucleic acids in the sequencing library. In some embodiments, the sample is not enriched for nucleic acids.

In some embodiments, the nucleic acids to be screened for repeat polymorphism are purified or isolated by any of a number of well-known methods.

Methods for isolating nucleic acids from biological samples are known in the art, and are dependent upon the type of nucleic acid being isolated (e.g., cfDNA, DNA, and/or RNA) and the type of sample from which the nucleic acids are being isolated (e.g., liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced and the sequencing technology being used.

For instance, many techniques for DNA isolation, e.g., genomic DNA isolation, from a tissue sample are known in the art, such as organic extraction, silica adsorption, and anion exchange chromatography. Likewise, many techniques for RNA isolation, e.g., mRNA isolation, from a tissue sample are known in the art. Non-limiting examples include acid guanidinium thiocyanate-phenol-chloroform extraction (see, for example, Chomczynski and Sacchi, 2006, Nat Protoc, 1(2):581-85, which is hereby incorporated by reference herein) and silica bead/glass fiber adsorption (see, for example, Poeckh, T. et al., 2008, Anal Biochem., 373(2):253-62, which is hereby incorporated by reference herein).

In some instances, it is advantageous to fragment the nucleic acid molecules in the nucleic acid sample. In some embodiments, fragmentation is random or specific, as achieved, for example, using restriction endonuclease digestion. Methods for random fragmentation are well known in the art, and include, for example, limited DNase digestion, alkali treatment and physical shearing.

9. Sequencing Library Preparation

In various embodiments, sequencing is performed on various sequencing platforms that require preparation of a sequencing library. For DNA, the preparation typically involves fragmenting the DNA (sonication, nebulization or shearing), followed by DNA repair and end polishing (blunt end or A overhang), and platform-specific adaptor ligation. In one embodiment, the methods described herein utilize NGS technologies that allow multiple samples to be sequenced individually as genomic molecules (e.g., singleplex sequencing) or as pooled samples comprising indexed genomic molecules (e.g., multiplex sequencing) on a single sequencing run. These methods can generate up to several hundred million reads of DNA sequences. In various embodiments the sequences of genomic nucleic acids, and/or of indexed genomic nucleic acids are determined using, for example, the NGS technologies described herein. In various embodiments, analysis of large data sets comprising sequence data obtained using NGS is performed using a system 100 and/or one or more processors as described herein.

Accordingly, in certain embodiments, the sequencing methods contemplated herein involve the preparation of sequencing libraries. In one illustrative approach, sequencing library preparation involves the production of a random collection of adapter-modified DNA fragments (e.g., polynucleotides) that are ready to be sequenced. Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. In some embodiments, the polynucleotides originate in double-stranded form (e.g., dsDNA such as genomic DNA fragments, cDNA, PCR amplification products, and the like) or, in certain embodiments, the polynucleotides originate in single-stranded form (e.g., ssDNA, RNA, etc.) and have been converted to dsDNA form. By way of illustration, in certain embodiments, single stranded mRNA molecules are copied into double-stranded cDNAs suitable for use in preparing a sequencing library. The precise sequence of the primary polynucleotide molecules is generally not material to the method of library preparation, and can be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.) that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

In some implementations, preparation of sequencing libraries for some NGS sequencing platforms is facilitated by the use of polynucleotides comprising a specific range of fragment sizes. Preparation of such libraries typically involves the fragmentation of large polynucleotides (e.g., cellular genomic DNA) to obtain polynucleotides in the desired size range. Paired end reads can be used for the methods and systems disclosed herein for determining repeat polymorphism. In alternative embodiments, the reads are single-end reads. In some implementations, the fragment or insert length is longer than the read length, and typically longer than the sum of the lengths of the two reads.

In some illustrative embodiments, the sample nucleic acid(s) are obtained as genomic DNA, which is subjected to fragmentation into fragments of approximately 100 or more, approximately 200 or more, approximately 300 or more, approximately 400 or more, or approximately 500 or more base pairs, and to which NGS methods can be readily applied. In some embodiments, the paired end reads are obtained from inserts of about 100-5000 bp. In some embodiments, the inserts are about 100-1000 bp long. In some embodiments, the inserts are about 1000-5000 bp long.

Fragmentation can be achieved by any of a number of methods known to those of skill in the art. For example, fragmentation can be achieved by mechanical means including, but not limited to nebulization, sonication and hydroshear. However, mechanical fragmentation typically cleaves the DNA backbone at C—O, P—O and C— bonds resulting in a heterogeneous mix of blunt and 3′- and 5′-overhanging ends with broken C—O, P—O and/C—C bonds (see, e.g., Alnemri and Liwack, J Biol. Chem 265:17323-17333 (1990); Richards and Boyer, J Mol Biol 11:327-240 (1965)), which, in some instances, need to be repaired as they lack the requisite 5′-phosphate for the subsequent enzymatic reactions, e.g., ligation of sequencing adaptors, that are required for preparing DNA for sequencing.

In contrast, cfDNA typically exists as fragments of less than about 300 base pairs and, consequently, fragmentation is not typically necessary for generating a sequencing library using cfDNA samples.

Typically, whether polynucleotides are forcibly fragmented (e.g., fragmented in vitro) or naturally exist as fragments, they are converted to blunt-ended DNA having 5′-phosphates and 3′-hydroxyl. Standard protocols (e.g., protocols for sequencing) instruct users to end-repair sample DNA, to purify the end-repaired products prior to dA-tailing, and to purify the dA-tailing products prior to the adaptor-ligating steps of the library preparation. Various embodiments of methods of sequence library preparation known to one of ordinary skill obviate the need to perform one or more of the steps typically mandated by standard protocols to obtain a modified DNA product that can be sequenced by NGS. The present methods and systems are contemplated to encompass such abbreviated methods.

In various embodiments, the use of such sequencing technologies does not involve the preparation of sequencing libraries.

10. Illustrative Sequencing Methods

In some embodiments, sequence reads are generated from nucleic acid molecules in a sample of the subject, where the nucleic acid molecules are optionally enriched, amplified, fragmented, isolated, and/or used to prepare a sequencing library or pool of sequencing libraries, as described above. In some embodiments, sequencing data is acquired by any methodology known in the art. For example, next-generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), and/or paired-end sequencing are contemplated for use in the present disclosure. In some embodiments, massively parallel sequencing is performed using sequencing-by-synthesis with reversible dye terminators. In some embodiments, sequencing is performed using next-generation sequencing technologies, such as short-read technologies. In other embodiments, long-read sequencing or another sequencing method known in the art is used.

In one illustrative, but non-limiting, embodiment, the methods described herein comprise obtaining sequence information for the nucleic acids in a test sample, using single molecule sequencing technology of the Helicos True Single Molecule Sequencing (tSMS) technology (e.g., as described in Harris T. D. et al., Science 320:106-109 (2008); Helicos, Inc., Cambridge, Mass.). In the tSMS technique, a DNA sample is cleaved into strands of approximately 100 to 200 nucleotides, and a poly-A sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In certain embodiments, the templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

In another illustrative, but non-limiting embodiment, the methods described herein comprise obtaining sequence information for the nucleic acids in the test sample, using the 454 sequencing (e.g., as described in Margulies, M. et al. Nature 437:376-380 (2005); (Roche, Basel, CH)). 454 sequencing typically involves two steps. In the first step, DNA is sheared into fragments of approximately 300-800 base pairs, and the fragments are blunt-ended. Oligonucleotide adaptors are then ligated to the ends of the fragments. The adaptors serve as primers for amplification and sequencing of the fragments. The fragments can be attached to DNA capture beads (e.g., streptavidin-coated beads) using, for instance, Adaptor B, which contains a 5′-biotin tag. The fragments attached to the beads are PCR amplified within droplets of an oil-water emulsion. The result is multiple copies of clonally amplified DNA fragments on each bead. In the second step, the beads are captured in wells (e.g., picoliter-sized wells). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is measured and analyzed.

In another illustrative, but non-limiting, embodiment, the methods described herein comprises obtaining sequence information for the nucleic acids in the test sample, using the SOLiD™ technology (Applied Biosystems, Waltham, Mass.). In SOLiD™ sequencing-by-ligation, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In another illustrative, but non-limiting, embodiment, the methods described herein comprise obtaining sequence information for the nucleic acids in the test sample, using the single molecule, real-time (SMRT™) sequencing technology of Pacific Biosciences (Menlo Park, Calif.). In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector comprises a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In another illustrative, but non-limiting embodiment, the methods described herein comprise obtaining sequence information for the nucleic acids in the test sample, using nanopore sequencing (e.g., as described in Soni G V and Meller A. Clin Chem 53: 1996-2001 (2007)). Nanopore sequencing DNA analysis techniques are developed by a number of companies, including, for example, Oxford Nanopore Technologies (Oxford, United Kingdom), Sequenom, NABsys, and the like. Nanopore sequencing is a single-molecule sequencing technology whereby a single molecule of DNA is sequenced directly as it passes through a nanopore. A nanopore is a small hole, typically of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore provides a read of the DNA sequence.

In another illustrative, but non-limiting, embodiment, the methods described herein comprises obtaining sequence information for the nucleic acids in the test sample, using the chemical-sensitive field effect transistor (chemFET) array (e.g., as described in U.S. Patent Application Publication No. 2009/0026082). In one example of this technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned as a change in current by a chemFET. An array can have multiple chemFET sensors. In another example, single nucleic acids can be attached to beads, and the nucleic acids can be amplified on the bead. The individual beads can be transferred to individual reaction chambers on a chemFET array, with each chamber having a chemFET sensor, and the nucleic acids can be sequenced.

In another illustrative, but non-limiting, embodiment, the DNA sequencing technology is the Ion Torrent (ThermoFisher Scientific, Waltham, Mass.) single molecule sequencing, which pairs semiconductor technology with a simple sequencing chemistry to directly translate chemically encoded information (A, C, G, T) into digital information (0, 1) on a semiconductor chip. In nature, when a nucleotide is incorporated into a strand of DNA by a polymerase, a hydrogen ion is released as a byproduct. Ion Torrent uses a high-density array of micro-machined wells to perform this biochemical process in a massively parallel way. Each well holds a different DNA molecule. Beneath the wells is an ion-sensitive layer and beneath that an ion sensor. When a nucleotide, for example a C, is added to a DNA template and is then incorporated into a strand of DNA, a hydrogen ion will be released. The charge from that ion will change the pH of the solution, which can be detected by Ion Torrent's ion sensor. The sequencer—essentially the world's smallest solid-state pH meter—calls the base, going directly from chemical information to digital information. The Ion personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be doubled, and the chip will record two identical bases called. Direct detection allows recordation of nucleotide incorporation in seconds.

In another illustrative, but non-limiting, embodiment, the present method comprises obtaining sequence information for the nucleic acids in the test sample, using sequencing by hybridization. Sequencing-by-hybridization comprises contacting the plurality of polynucleotide sequences with a plurality of polynucleotide probes, where each of the plurality of polynucleotide probes can be optionally tethered to a substrate. The substrate can be a flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be determined and used to identify the plurality of polynucleotide sequences within the sample.

In some embodiments of the methods described herein, the sequencing generates a set of sequence reads. In some embodiments, each respective sequence read in the set of sequence reads is at least about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp in length. In some embodiments, each respective sequence read in the set of sequence reads is of a predetermined length. In some embodiments, each respective sequence read in the set of sequence reads is from about 50 bp to about 200 bp. In some embodiments, each respective sequence read in the set of sequence reads is about 100 bp in length.

In some embodiments, the genomic locus having the repeat sequence polymorphism is longer than each respective sequence read in the set of sequence reads. For instance, in some embodiments, the genomic locus having the repeat sequence polymorphism is longer than about 100 bp, 500 bp, 1000 bp, or 4000 bp.

11. Alignment to Reference Genome

After sequencing of nucleic acids (e.g., DNA fragments), the resulting sequence reads are mapped or aligned to a known reference sequence or reference genome. In some implementations, the mapped or aligned reads and their corresponding locations on the reference sequence are referred to as tags or sequence tags. Alternatively or additionally, in some embodiments, the systems and methods disclosed herein (e.g., determining genotypes for repeat sequence polymorphisms, such as repeat expansions or deletions) make use of sequence reads that are either poorly aligned or cannot be aligned, as well as aligned reads (tags).

In some embodiments, the reference sequence is the NCBI36/hg18 sequence, which is available on the Internet at ncbi.nlm.nih.gov/assembly/GCF_000001405.39. Alternatively, in some embodiments, the reference genome sequence is the GRCh37/hd19, which is available on the Internet at ncbi.nlm.nih.gov/assembly/GCF_000001405.13/. Other sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al., Genome Biology 10:R25.1-R25.10 [2009]), and/or ELAND (Illumina, Inc., San Diego, Calif., USA). In some embodiments, one end of the clonally expanded copies of the plasma cfDNA molecules is sequenced and processed by bioinformatic alignment analysis for the Illumina Genome Analyzer, which uses the Efficient Large-Scale Alignment of Nucleotide Databases (ELAND) software.

In some implementations, mapping of the sequence reads is achieved by comparing the sequence of the sequence reads with the sequence of the reference sequence to determine the chromosomal origin of the sequenced nucleic acid molecule, and specific genetic sequence information is not needed. In some implementations, a small degree of mismatch (0-2 mismatches per read) is allowed to account for minor polymorphisms that can exist between the reference genome and the genome in the sample. In some embodiments, poorly aligned reads can have a relatively large number of percentage of mismatches per read, e.g., at least about 5%, at least about 10%, at least about 15%, or at least about 20% mismatches per read.

A plurality of sequence tags (e.g., reads aligned to a reference sequence) are typically obtained per sample. In some embodiments, at least about 3×10⁶ sequence tags, at least about 5×10⁶ sequence tags, at least about 8×10⁶ sequence tags, at least about 10×10⁶ sequence tags, at least about 15×10⁶ sequence tags, at least about 20×10⁶ sequence tags, at least about 30×10⁶ sequence tags, at least about 40×10⁶ sequence tags, or at least about 50×10⁶ sequence tags of, e.g., 100 bp, are obtained from mapping the reads to the reference genome per sample. In some embodiments, all the sequence reads are mapped to all regions of the reference genome, providing genome-wide reads. In other embodiments, sequence reads are mapped to a sequence of interest, e.g., a genomic locus, a chromosome, a segment of a chromosome, or a repeat sequence of interest.

12. Therapeutic Agents

Any suitable therapeutic agent can be used in conjunction with the systems and methods described herein. In some embodiments the therapeutic agent is a single therapeutic agent. In other embodiments, the therapeutic agent includes 2, 3, 4, 5, 6, 7, 8, 9, or 10 therapeutic agents.

Suitable therapeutic agents include, but are not limited to, molecular inhibitors, antibodies, recombinant nucleic acids (e.g., antisense oligonucleotides) and engineered immune cells (e.g., CAR T-cells and NK cells). Exemplary therapeutic agents include, but are not limited to, Paclitaxel, Gemcitabine, Cisplatin, Carboplatin, Oxaliplatin, Capecitabine, SN-38 (CPT-11), 5-FU, MTX (methotrexate), Docetaxel, Bortezomib, Everolimus, Ulixertinib, Dasatinib, Vinblastine, Nelarabine, Epirubicin, Afatinib, Lapatinib, Cytarabine, Cladribine, Doxorubicin, Azacitidine, and/or Staurosporine. Other examples include classes of drugs including but not limited to: taxanes, platinating agents, vinca alkaloids, alkylating agents, and/or anthracyclines.

In some embodiments, the one or more therapeutic agents include one or more of the following: an inhibitor of SUV4-20 (SUV420H1 or SUV420H2), a tyrosine kinase inhibitor, a retinoid-like compound, a weel kinase inhibitor, an anaplastic lymphoma kinase inhibitor, an aurora A kinase inhibitor, an aurora B kinase inhibitor, a reversible inhibitor of eukaryotic nuclear DNA replication, an antimetabolite antineoplastic agent, an ataxia telangiectasia and Rad3-related protein (ATR) kinase inhibitor, an ATM kinase inhibitor, a checkpoint kinase inhibitor, a GSK-3a/b inhibitor, a proteasome inhibitor, an AXL or RET inhibitor, a c-Met or VEGFR2 inhibitor, an alkylating antineoplastic agent, a DNA-PK and/or mTOR inhibitor, an inhibitor of mammalian target of rapamycin (mTOR), a checkpoint kinase 1 (CHK1) inhibitor, a retinoic acid receptor β (RARβ) or RARγ antagonist, a retinoic acid receptor (RAR)γ-selective agonist, RARγ-selective retinoid, inducer of apoptosis, CDK2 a RAR agonist, a chemotherapy, a tyrosine kinase inhibitor antineoplastic agent, an antimicrotubular antineoplastic agent, a topoisomerase inhibitor antineoplastic agent, a sodium-glucose cotransporter-2/SGLT2 inhibitor, an inhibitor of the tropomyosin receptor kinases A, B and C, C-ros oncogene 1 and anaplastic lymphoma kinase, a topoisomerase inhibitor antineoplastic agent, an inhibitor of mTOR, an inhibitor of phosphatidylinositol 3-kinase (PI3K), an inhibitor of RIP3K, an analog of cyclophosphamide, an SGLT2 inhibitor, aWnt/β-catenin inhibitor, a tyrosine kinase inhibitor that interrupts the HER2/neu and epidermal growth factor receptor/EGFR pathways, an inhibitor of tropomyosin kinase receptors TrkA, TrkB, and TrkC, a cyclin-dependent kinase (CDK) inhibitor, a CDK7 inhibitor, an inhibitor of VEGFR1, VEGFR2 and VEGFR3 kinases, a DNA-PK/PI3K/mTOR inhibitor, a poly ADP ribose polymerase (PARP) inhibitor, an inhibitor of Rac GTPase, a taxane, a Bromodomain And PHD Finger Containing 1 (BRPF1) bromodomain inhibitor, a mitogen-activated protein kinase-activated protein kinase 2 (MAPK2) inhibitor, a RAF inhibitor, a histone deacetylase (HDAC) inhibitor, a CDK1 inhibitor, a TGF-beta/Smad inhibitor, a Pim kinase inhibitor, a DNA topoisomerase I inhibitor, active metabolite of CPT-11/Irinotecan, an atypical retinoid, apoptosis inducer, a multi-kinase inhibitor, a fms-like tyrosine kinase-3 (FLT3) inhibitor, a MEK inhibitor, an inhibitor of extracellular signal-regulated kinase (ERK) 1 and/or 2, and/or a DNA-dependent protein kinase/DNA-PK inhibitor.

In some embodiments, the one or more therapeutic agents include one or more of the following: A-196 (inhibitor of SUV4-20 or SUV420H1 and SUV420H2), Afatinib (tyrosine kinase inhibitor), Adapalene (retinoid-like compound), Adavosertib (MK-1775, weel kinase inhibitor), Alectinib (CH5424802, anaplastic lymphoma kinase inhibitor), Alisertib (MLN8237, aurora A kinase inhibitor), Aphidicolin (reversible inhibitor of eukaryotic nuclear DNA replication, antimitotic), Azacitidine (an antimetabolite antineoplastic agent, a chemotherapy), AZ20 (ataxia telangiectasia and Rad3-related protein/ATR kinase inhibitor), AZ31 (ataxia-telangiectasia mutated/ATM kinase inhibitor), AZD6738 (ataxia telangiectasia and Rad3-related protein/ATR kinase inhibitor), AZD7762 (checkpoint kinase inhibitor), Barasertib (AZD1152-HQPA, aurora B kinase inhibitor), BAY-1895344 (ATR and ATM kinase inhibitor), Berzosertib (ATR and ATM kinase inhibitor), BIO-acetoxime (GSK-3a/b inhibitor), Bortezomib (proteasome inhibitor), Cabozantinib (kinase inhibitor, inhibitor of AXL, RET, and tyrosine kinases c-Met and VEGFR2), Capecitabine (an antimetabolite antineoplastic agent, a chemotherapy), Carboplatin (an alkylating antineoplastic agent, a chemotherapy), CC-115 (DNA-PK and mTOR inhibitor), CC-223 (inhibitor of mammalian target of rapamycin/mTOR), CCT-245737 (checkpoint kinase 1/CHK1 inhibitor), CD-2665 (retinoic acid receptor β (RARβ)/RARγ antagonist), CD-437 (retinoic acid receptor (RAR)γ-selective agonist, γ-selective retinoid; inducer of apoptosis), CDK2 inhibitor II, CH-55 (RAR agonist), Cisplatin (an alkylating antineoplastic agent, a chemotherapy), Cladribine (an antimetabolite antineoplastic agent, a chemotherapy), Cytarabine (an antimetabolite antineoplastic agent, a chemotherapy), Dasatinib (a tyrosine kinase inhibitor antineoplastic agent, a chemotherapy), Docetaxel (an antimicrotubular antineoplastic agent, a chemotherapy), Doxorubicin (Adriamycin, a topoisomerase inhibitor antineoplastic agent, a chemotherapy), Empagliflozin (BI 10773, a sodium-glucose cotransporter-2/SGLT2 inhibitor), Entrectinib (RXDX-101, tyrosine kinase inhibitor, inhibitor of the tropomyosin receptor kinases A, B and C, C-ros oncogene 1 and anaplastic lymphoma kinase), Epirubicin (a topoisomerase inhibitor antineoplastic agent, a chemotherapy), Etoposide (a topoisomerase inhibitor antineoplastic agent, a chemotherapy), Everolimus (inhibitor of mTOR), Fluorouracil/5-FU (an antimetabolite antineoplastic agent, a chemotherapy), GDC-0349 (inhibitor of mTOR), GDC-0575 (ARRY-575, CHK1 inhibitor), Gemcitabine (an antimetabolite antineoplastic agent, a chemotherapy), GSK2292767 (inhibitor of phosphatidylinositol 3-kinase/PI3K), GSK-872 (GSK2399872A, kinase inhibitor, inhibitor of RIP3K), Hesperadin (aurora kinase inhibitor), Hydroxyurea (an antimetabolite antineoplastic agent, a chemotherapy), Ifosfamide (an analog of cyclophosphamide, an alkylating antineoplastic agent, a chemotherapy), Ipragliflozin (ASP1941, an SGLT2 inhibitor), KYA1797K (Wnt/β-catenin inhibitor), Lapatinib (tyrosine kinase inhibitor that interrupts the HER2/neu and epidermal growth factor receptor/EGFR pathways, an antineoplastic agent, a chemotherapy), Larotrectinib (inhibitor of tropomyosin kinase receptors TrkA, TrkB, and TrkC), LDC 4297 (Cyclin-dependent kinase/CDK inhibitor, CDK7 inhibitor), Lenvatinib (multiple kinase inhibitor, inhibitor of VEGFR1, VEGFR2 and VEGFR3 kinases), LY3023414 (DNA-PK/PI3K/mTOR Inhibitor), Methotrexate (an antimetabolite antineoplastic agent, a chemotherapy), Nelarabine (an antimetabolite antineoplastic agent, a chemotherapy), Niraparib (MK-4827, a poly ADP ribose polymerase/PARP inhibitor), NSC 23766 (inhibitor of Rac GTPase), Olaparib (PARP inhibitor), Oxaliplatin (an alkylating antineoplastic agent, a chemotherapy), Paclitaxel (a taxane, an antimicrotubular antineoplastic agent, a chemotherapy), Pamiparib (BGB-290, PARP inhibitor), PFI-4 (Bromodomain And PHD Finger Containing 1/BRPF1 bromodomain inhibitor), PHA-767491 HCl (Mitogen-activated protein kinase-activated protein kinase 2/MK2 and CDK inhibitor), PLX7904 (RAF inhibitor), Pracinostat (histone deacetylase/HDAC inhibitor), Pralatrexate (an antimetabolite antineoplastic agent, a chemotherapy), Prexasertib HCl (checkpoint kinase 1/CHK1 inhibitor), RO-3306 (CDK1 inhibitor), Rucaparib (PARP inhibitor), Selpercatinib (LOXO-292, ARRY-192, a tyrosine kinase inhibitor), SIS3 HCl (TGF-beta/Smad inhibitor), SMI-4a (Pim kinase inhibitor), SN-38 (inhibitor of DNA topoisomerase I, active metabolite of CPT-11/Irinotecan), ST-1926 (Adarotene, atypical retinoid, apoptosis inducer), Staurosporine (multi-kinase inhibitor used as a positive control), Talazoparib (BMN-673, PARP inhibitor), TCS 359 (fms-like tyrosine kinase-3/FLT3 inhibitor), Tenalisib (RP6530, a PI3K δ/γ inhibitor), Tozasertib (VX-680, MK-0457, an Aurora Kinase inhibitor), Trametinib (GSK1120212, a MEK inhibitor), Ulixertinib (inhibitor of extracellular signal-regulated kinase/ERK 1 and 2, with potential antineoplastic activity), Veliparib (ABT-888, PARP inhibitor), Vinblastine (an antimicrotubular antineoplastic agent, a chemotherapy), and/or VX-984 (DNA-dependent protein kinase/DNA-PK inhibitor).

In some embodiments, the one or more therapeutic agents include one or the following therapeutic agents or combination therapeutics: afatinib plus MET inhibitor (for example, tivantinib, cabozantinib, crizotinib, etc.), AZ31 plus SN-38, bevacizumab (anti-VEGF monoclonal IgG1 antibody), cetuximab (epidermal growth factor receptor/EGFR inhibitor), crizotinib (a tyrosine kinase inhibitor antineoplastic agent), cyclophosphamide (an alkylating antineoplastic agent), erlotinib (epidermal growth factor receptor inhibitor antineoplastic agent), FOLFIRI, bevacizumab plus FOLFIRI, FOLFOX, gefitinib (EGFR inhibitor), gemcitabine plus docetaxel, pemtrexed (an antimetabolite antineoplastic agent), ramucirumab (Vascular Endothelial Growth Factor Receptor 2/VEGFR2 Inhibitor), and/or topotecan (a topoisomerase inhibitor).

### E. Additional Embodiments

Another aspect of the present disclosure provides a computer system comprising one or more processors, memory, and one or more programs, where the one or more programs are stored in the memory and are configured to be executed by the one or more processors, the one or more programs including instructions for determining a genotype of a subject at a genomic locus comprising a tandem repeat, from a plurality of candidate genotypes for the genomic locus.

The method comprises obtaining, in electronic form, a first set of sequence reads obtained from a biological sample of the subject that map to the tandem repeat in the genomic locus, where the tandem repeat consists of a plurality of contiguous nucleotide repeat units and each respective sequence read in the first set of sequence reads encompasses the tandem repeat. The method further includes determining, for each respective sequence read in the first set of sequence reads, a corresponding repeat count of the number of repeat units in the plurality of contiguous repeat units in the respective sequence read, thereby determining a distribution of repeat counts of the number of repeat units in the first set of sequence reads.

The method further includes obtaining a plurality of sets of repeat count adjustment factors, where each respective set of repeat count adjustment factors corresponds to a candidate allele in a plurality of candidate alleles, each respective candidate allele in the plurality of candidate alleles has a different corresponding number of repeat units for the plurality of contiguous nucleotide repeat units, each respective set of repeat count adjustment factors includes a corresponding repeat count adjustment factor for each respective number of repeat units in a numerical range of repeat units for the plurality of contiguous nucleotide repeat units, and each combination of two respective candidate alleles in the plurality of candidate alleles corresponds to a respective candidate genotype in the plurality of candidate genotypes.

The method further comprises assigning, for each respective candidate genotype in the plurality of candidate genotypes, a corresponding likelihood for the respective candidate genotype based, at least in part, upon, for each respective candidate allele corresponding to the respective candidate genotype: (i) a proportion of sequence reads in the plurality of sequence reads that have the repeat count of the number of repeat units in the plurality of contiguous repeat units corresponding to the respective candidate allele, and (ii) a repeat count adjustment factor matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles. The method further comprises selecting the respective candidate genotype in the plurality of candidate genotypes having the highest corresponding likelihood.

Still another aspect of the present disclosure provides a computer readable storage medium storing one or more programs, the one or more programs comprising instructions that, when executed by an electronic device with one or more processors and a memory, cause the electronic device to perform a method for determining a genotype of a subject at a genomic locus comprising a tandem repeat, from a plurality of candidate genotypes for the genomic locus.

The method comprises obtaining, in electronic form, a first set of sequence reads obtained from a biological sample of the subject that map to the tandem repeat in the genomic locus, where the tandem repeat consists of a plurality of contiguous nucleotide repeat units and each respective sequence read in the first set of sequence reads encompasses the tandem repeat. The method further includes determining, for each respective sequence read in the first set of sequence reads, a corresponding repeat count of the number of repeat units in the plurality of contiguous repeat units in the respective sequence read, thereby determining a distribution of repeat counts of the number of repeat units in the first set of sequence reads.

The method further includes obtaining a plurality of sets of repeat count adjustment factors, where each respective set of repeat count adjustment factors corresponds to a candidate allele in a plurality of candidate alleles, each respective candidate allele in the plurality of candidate alleles has a different corresponding number of repeat units for the plurality of contiguous nucleotide repeat units, each respective set of repeat count adjustment factors includes a corresponding repeat count adjustment factor for each respective number of repeat units in a numerical range of repeat units for the plurality of contiguous nucleotide repeat units, and each combination of two respective candidate alleles in the plurality of candidate alleles corresponds to a respective candidate genotype in the plurality of candidate genotypes.

The method further comprises assigning, for each respective candidate genotype in the plurality of candidate genotypes, a corresponding likelihood for the respective candidate genotype based, at least in part, upon, for each respective candidate allele corresponding to the respective candidate genotype: (i) a proportion of sequence reads in the plurality of sequence reads that have the repeat count of the number of repeat units in the plurality of contiguous repeat units corresponding to the respective candidate allele, and (ii) a repeat count adjustment factor matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles. The method further comprises selecting the respective candidate genotype in the plurality of candidate genotypes having the highest corresponding likelihood.

Yet another aspect of the present disclosure provides a computer system having one or more processors, and memory storing one or more programs for execution by the one or more processors, the one or more programs comprising instructions for performing any of the methods and/or embodiments disclosed herein. In some embodiments, any of the presently disclosed methods and/or embodiments are performed at a computer system having one or more processors, and memory storing one or more programs for execution by the one or more processors.

Still another aspect of the present disclosure provides a non-transitory computer readable storage medium storing one or more programs configured for execution by a computer, the one or more programs comprising instructions for carrying out any of the methods disclosed herein.

### F. Further Embodiments

The following clauses describe specific embodiments of the disclosure.

Clause 1. A method for determining the presence or absence of one or more repeat sequence polymorphisms in a test sample comprising nucleic acids, wherein the repeat sequence genotype comprises a varying number of repeats of a repeat unit of nucleotides, the method comprising: (a) sequencing, using a nucleic acid sequencer, the test sample to obtain sequencing reads; (b) aligning, using a computer system comprising one or more processors and system memory, the sequencing reads to a reference genome comprising the repeat sequence; (c) comparing, by the one or more processors, genomic locations of the reads to the genomic location of the repeat sequence to identify those having genomic locations that are the same as or near the genomic location of the repeat sequence to provide mapped reads; (d) realigning the mapped reads to a linear graph model of the repeat sequence genotypes of interest; (e) counting the number of alignments for each genotype; and (f) applying a caller using the number of alignments to determine the probable identity of the repeat sequence genotypes at each allele within the test sample to provide a genotype call, wherein the genotype call is evidence of the likely presence of a particular polymorphism of a repeat sequence genotype in the test sample.

Clause 2. The method of Clause 1, wherein the method further comprises: (g) applying an error model that accounts for DNA polymerase stutter to determine a confidence value for the genotype call; and (h) utilizing the confidence values to filter out low quality calls, wherein the remaining calls provide evidence for the presence of a particular polymorphism of a repeat sequence genotype.

Clause 3. The method of Clause 1, further comprising, upon determination that a polymorphism of interest is likely present in the test sample, performing an additional analysis to determining if the test sample comprises a particular repeat polymorphism.

Clause 4. The method of Clause 1, wherein the additional analysis comprises assaying the test sample using longer reads.

Clause 5. The method of Clause 4 wherein the additional analysis comprises using single molecule sequencing or using synthetic long-read sequencing.

Clause 6. The method of Clause 1, wherein the mapped reads are aligned to or within about 5 kb of the repeat sequence.

Clause 7. The method of Clause 1, wherein the mapped reads are aligned to or within about 1 kb of the repeat sequence.

Clause 8. The method of Clause 1, further comprising determining that an individual from whom the test sample is obtained has an elevated risk of side effects with the administration of a chemotherapeutic drug.

Clause 9. The method of Clause 8, wherein the chemotherapeutic drug is one that is metabolized by the enzyme product of the UDP glucuronosyltransferase (UGT)1A1 gene.

Clause 10. The method of Clause 9, wherein the chemotherapeutic drug is selected from the group consisting of abiraterone, acalabrutinib, asciminib, anastrozole, axitinib, belinostat, bendamustin, bexarotene, bicalutamide, binimetinib, bleomycin, camptothecin, cerdulatinib, chlorambucil, cobimetinib, cytarabine, dasatinib, daunorubicin, doxorubicin, duvelisib, enasidenib, encorafenib, epirubicin, erlotinib, etoposide, exemestane, fenretinide, flavopiridol, fludarabine, 5-fluorouracil, fluoxymesterone, flumatinib, fostamatinib, fulvestrant, glasdegib, ibrutinib, idelalisib, imatinib, irinotecan, isotretinoin, larotrectinib, letrozole, lorlatinib, medroxyprogesterone, megestrol, methotrexate, mitoxantrone, nintedanib, niraparib, olaparib, palbociclib, panobinostat, pomalidomide, raloxifene, regorafenib, ribavirin, ruxolitinib, selinexor, sorafenib, sunitinib, talazoparib, tamoxifen, thalidomide, tipifarnib, topotecan, toremifene, trabectedin, trametinib, tretinoin, vandetanib, venetoclax, vismodegib, and vorinostat.

Clause 11. The method of Clause 1, further comprising determining that an individual from whom the test sample is obtained has an elevated risk of one of Fragile X syndrome, amyotrophic lateral sclerosis (ALS), Huntington's disease, Friedreich's ataxia, spinocerebellar ataxia, spino-bulbar muscular atrophy, myotonic dystrophy, Machado-Joseph disease, or dentatorubral pallidoluysian atrophy.

Clause 12. The method of Clause 1, wherein the test sample is a blood sample, a urine sample, a saliva sample, or a tissue sample.

Clause 13. The method of Clause 1, wherein the test sample comprises fetal and maternal cell-free nucleic acids.

Clause 14. A method, implemented using a computer system comprising one or more processors and system memory, for determining the presence or absence of a repeat sequence polymorphism in a test sample comprising nucleic acids, wherein the repeat sequence genotype comprises a varying number of repeats of a repeat unit of nucleotides, the method comprising: (a) sequencing, using a nucleic acid sequencer, the test sample to obtain sequencing reads; (b) aligning the sequencing reads to a reference genome comprising the repeat sequence; (c) comparing genomic locations of the reads to the genomic location of the repeat sequence to identify those having genomic locations that are not same as or near the genomic location of the repeat sequence to provide unmapped reads; (d) applying a Bayesian caller using the number of non-aligned reads to determine the likely presence of a particular polymorphism of a repeat sequence genotype in the test sample.

Clause 15. The method of Clause 14, further comprising: (g) applying an error model that accounts for DNA polymerase stutter to determine a confidence value for the genotype call; and (h) utilizing the confidence values to filter out low quality calls, wherein the remaining calls provide evidence for the presence of a particular polymorphism of a repeat sequence genotype.

Clause 16. The method of Clause 14, further comprising, upon determination that a polymorphism of interest is likely present in the test sample, performing an additional analysis to determining if the test sample comprises a particular repeat polymorphism.

Clause 17. The method of Clause 16, wherein the additional analysis comprises assaying the test sample using longer reads.

Clause 18. The method of Clause 16, wherein the additional analysis comprises using single molecule sequencing or using synthetic long-read sequencing.

Clause 19. A system comprising one or more processors and system memory for determining the presence or absence of a repeat sequence polymorphism in a test sample comprising nucleic acids, wherein the repeat sequence genotype comprises a varying number of repeats of a repeat unit of nucleotides, the system configured to: (a) sequence, using a nucleic acid sequencer, the test sample to obtain sequencing reads; (b) align the sequencing reads to a reference genome comprising the repeat sequence; (c) compare genomic locations of the reads to the genomic location of the repeat sequence to identify those having genomic locations that are the same as or near the genomic location of the repeat sequence to provide mapped reads; (d) realign the mapped reads to a linear graph model of the repeat sequence genotypes of interest; (e) count the number of alignments for each genotype; and (f) apply a Bayesian caller using the number of alignments to determine the probable identity of the repeat sequence genotypes at each allele within the test sample to provide a genotype call, wherein the genotype call is evidence of the presence of a particular polymorphism of a repeat sequence genotype in the test sample.

Clause 20. The system of Clause 19, wherein the system further configured to: (g) apply an error model that accounts for DNA polymerase stutter to determine a confidence value for the genotype call; and (h) utilize the confidence values to filter out low quality calls, wherein the remaining calls provide evidence for the presence of a particular polymorphism of a repeat sequence genotype.

### EXAMPLES

### Example 1—Accurate Genotyping of UGT1A1 Dinucleotide Repeat Polymorphism from Targeted NGS Data for the Assessment of Irinotecan Chemotherapy Adverse Events

Irinotecan (IRI) is commonly used to treat metastatic colorectal cancer (CRC). The gene UGT1A1 encodes the enzyme responsible for the glucuronidation of SN-38, the active metabolite of IRI. The TA repeat in the promoter region of UGT1A1 is highly polymorphic. Wild-type UGT1A1 contains six TA repeats [A(TA)₆TAA]. Polymorphic UGT1A1 alleles with a higher number of TA repeats, such as UGT1A1*28/(TA)₇ and *37/(TA)₈ alleles, decrease promoter activity and are associated with severe toxicity in patients receiving IRI-based chemotherapy. An experimental assay was performed to determine the utility of matched tumor/normal genomic profiling by NGS for cancer therapy in assessing therapy-induced adverse events due to germline variants. However, genotyping of UGT1A1 polymorphisms is commonly carried out with PCR or fragment analysis in capillary electrophoresis, and not from NGS data. This is due, at least in part, to challenges in aligning short reads to repeats and the introduction of “stutter” artifacts. See, e.g., Raz et al., “Short tandem repeat stutter model inferred from direct measurement of in vitro stutter noise,” Nucleic Acids Res 47, gky1318 (2019).

Here, an example method for calling accurate UGT1A1 TA repeat genotypes from target capture NGS data is described, and the feasibility of this method for genomic profiling of cancer patients is demonstrated.

Methods

An overview of the UGT1A1 analysis workflow will now be described, in accordance with an embodiment of the present disclosure. In some embodiments, the UGT1A1 analysis workflow is performed in accordance with the example workflows illustrated in FIGS. 4A-C. A first plurality of sequence reads was obtained and aligned to a reference sequence including a genomic locus corresponding to the UGT1A1 gene sequence. Sequence reads that mapped to a tandem repeat in the genomic locus corresponding to the UGT1A1 promoter were selected, thereby obtaining a first set of sequence reads. Sequence reads in the first set of sequence reads were deduplicated, and the deduplicated reads were realigned to a graph-based model representing the possible candidate alleles, where each linear model corresponded to a possible repeat count for a number of repeat units in the TA repeat sequence in the UGT1A1 promoter region.

FIGS. 5A-F illustrate example realignments of sequence reads spanning a TA repeat sequence to linear graph models representing a set of different possible numbers of repeat units in the TA repeat sequence. For instance, the example linear graph models include representations of candidate alleles having 5, 6, 7, 8, 9, and 10 repeated TA units (e.g., (TA)₅, (TA)₆, (TA)₇, (TA)₈, (TA)₉, and (TA)₁₀). In the UGT1A1 workflow, repeat-spanning read alignments produced by an initial mapping technique (BWA) were de-duplicated and realigned locally to several models of the reference including the different repeat lengths expected. These alignments provided read counts that indicated the number of sequence reads in the first set of sequence reads that corresponded to (e.g., aligned to) a particular candidate allele having a respective repeat count of the number of repeat units in the tandem repeat (e.g., 5, 6, 7, 8, 9, and 10).

Referring again to the UGT1A1 analysis workflow, genotype calling was performed using a Bayesian model. The set of hypotheses to be tested using the Bayesian model included all possible genotypes at the genomic locus, under a consideration of homozygosity or heterozygosity. Thus, for instance, a respective hypothesis could be “5/5,” where the sample is homozygous with both alleles having 5 repeats. In another example, a respective hypothesis could be “6/8,” where the sample is heterozygous with one allele having 6 repeats and the other allele having 8 repeats.

The Bayesian model tested each possible genotype (e.g., hypothesis) using a plurality of repeat count adjustment factors, where the plurality of repeat count adjustment factors was obtained using an empirically derived DNA polymerase stutter model (e.g., an empirically derived distribution of the probabilities of observing various repeat counts in sequence reads, given a particular haplotype in an originating sample). More particularly, the plurality of repeat count adjustment factors included a respective set of adjustment factors for each respective candidate allele in a plurality of candidate alleles, where each respective candidate allele had a different respective corresponding number of repeat units in the tandem repeat sequence (e.g., a different haplotype). Moreover, each respective set of adjustment factors included a corresponding adjustment factor for each respective number of repeat units in a numerical range of repeat units (e.g., a different repeat count in a range of potentially observable repeat counts for a given sequence read in the first set of sequence reads).

To generate the plurality of repeat count adjustment factors, the stutter model was derived from empirical observations in 1,419 patient blood samples sequenced with a 648-gene, targeted panel NGS assay on tumor-normal matched samples (hereinafter, “xT assay”). As illustrated in FIG. 9, the empirically observed stutter distribution was also well approximated by a simple one-parameter (probability of TA insertions) error model. Advantageously, the simple error model was capable of generalizing to arbitrary repeat lengths for which empirical data were not available. This model was later used to construct prior probabilities when evaluating genotype hypotheses using the Bayesian model.

Referring again to the UGT1A1 analysis workflow, the Bayesian model provided genotype calls and quality metrics (e.g., posterior probabilities), which were used to eliminate genotyping errors for poor quality data and/or samples. Assigning probabilities to candidate alleles and genotype hypotheses are described in greater detail elsewhere herein (see, e.g., the section entitled “Assigning likelihood of candidate genotypes,” above). An example illustration of using read counts to determine genotype calls using a Bayesian model is provided in Tables 4 and 5, with reference to Equation 2.

TABLE 4
Example Read Counts

| Repeat Length | Read Count |
|---|---|
| 5 | 2 |
| 6 | 16 |
| 7 | 140 |
| 8 | 116 |
| 9 | 23 |
| 10 | 2 |

Applying Equation 2 to the read counts displayed in Table 4, the posterior probabilities and corresponding quality metrics shown in Table 5 can be obtained.

$$P ( H \vert E ) = \frac{P ( E \vert H ) P ( H )}{P ( E )} , \qquad [ Equation 2 ]$$

TABLE 5
Example Genotype Call from Read Counts

| Hypothesis Scored | | Log Posterior | Log Odds | |
|---|---|---|---|---|
| Allele A | Allele B | Probability | Ratio | GQ |
| 5 | 5 | −2930.28 | | |
| 5 | 6 | −1737.44 | | |
| 5 | 7 | −782.39 | | |
| 5 | 8 | −746.62 | | |
| 5 | 9 | −1391.93 | | |
| 5 | 10 | −1998.13 | | |
| 6 | 6 | −1539.04 | | |
| 6 | 7 | −741.68 | | |
| 6 | 8 | −624.57 | | |
| 6 | 9 | −956.98 | | |
| 6 | 10 | −1343.19 | | |
| 7 | 7 | −596.34 | | |
| 7 | 8 | −365.37 | 230.9 | 1003 |
| 7 | 9 | −538.94 | | |
| 7 | 10 | −675.33 | | |
| 8 | 8 | −610.47 | | |
| 8 | 9 | −730.81 | | |
| 8 | 10 | −781.31 | | |
| 9 | 9 | −1356.24 | | |
| 9 | 10 | −1545.22 | | |
| 10 | 10 | −2335.41 | | |

To benchmark the method described herein, alignment data for TA repeat lengths ranging from 4 to 10 copies was simulated for multiple genotype combinations and coverage depths using sequencing data from 224 patient blood samples sequenced with the xT assay described above (deduplicated coverage ranging 200-500×). The simulation was constructed by partitioning the alignments present in the full set of available samples based on their BAM alignments CIGAR string. From these available reads, it was possible to simulate samples containing alleles that were not actually expected to be seen in the population (e.g., (TA)₅ or (TA)₁₀), and thus better evaluate the behavior of the genotype calling algorithm.

Results

As described above, alignment data were simulated for various combinations of candidate alleles having different numbers of repeat units in the tandem repeat sequence (e.g., different repeat lengths), thus generating different genotype combinations for testing (“Truth”). The data were simulated at both 100× and 500× depths of coverage. Genotype calls and quality metrics were then made in accordance with the methods disclosed herein. Genotype calls (“Call”) and genotype quality scores (“GQ”) for both coverages are shown in Table 6, demonstrating the ability to correctly call known alleles as well as new potential alleles without misclassification.

TABLE 6
Performance of Genotype Caller with
Simulated Data

| | 100× | | 500× | |
|---|---|---|---|---|
| Truth | Call | GQ | Call | GQ |
| 5/5 | 5/5 | 285 | 5/5 | 1242 |
| 6/6 | 6/6 | 218 | 6/6 | 1082 |
| 7/7 | 7/7 | 206 | 7/7 | 1039 |
| 8/8 | 8/8 | 203 | 8/8 | 990 |
| 9/9 | 9/9 | 192 | 9/9 | 956 |
| 10/10 | 10/10 | 273 | 10/10 | 1349 |
| 5/7 | 5/7 | 539 | 5/7 | 2618 |
| 6/7 | 6/7 | 342 | 6/7 | 1673 |
| 7/8 | 7/8 | 324 | 7/8 | 1651 |
| 7/9 | 7/9 | 472 | 7/9 | 2346 |
| 7/10 | 7/10 | 501 | 7/10 | 2500 |
| 6/9 | 6/9 | 493 | 6/9 | 2466 |
| 5/10 | 5/10 | 588 | 5/10 | 2812 |

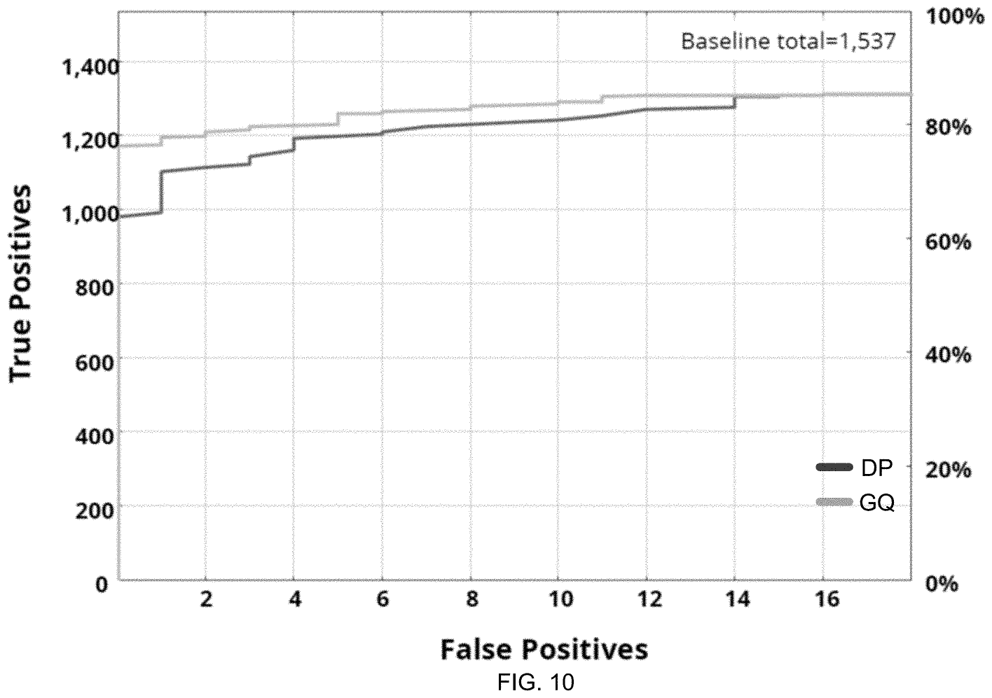

As described above, in some embodiments, one or more determined genotypes (e.g., genotype calls) are filtered based on one or more quality metrics. A receiver-operator curve in FIG. 10 shows the discriminating ability of two possible quality scores: read depth (“DP,” black) and Phred-scaled genotype quality (“GQ,” gray). Based on these results, a GQ of 70 was selected as a cutoff threshold to filter raw variant calls and remove false positives.

An analysis was performed to explore the dependency of the Bayesian caller's ability to call candidate alleles on the depth of coverage. A method for genotype calling was performed in accordance with the present disclosure using germline data from the set of 224 patient samples sequenced with the tumor-normal matched xT NGS test, which targeted 648 cancer-related genes including the UGT1A1 promoter. The UGT1A1 candidate alleles in those samples were determined by an orthogonal method that searched for patterns in unaligned reads (“the silver set”). By subsampling the data, several levels of read depth were simulated. No GQ filter was used in this analysis. As shown in Table 7 and highlighted above, the results indicate that a minimum of 70× is necessary for accurate results. In particular, it was observed that with a minimum depth of 70×, 100% accuracy and robustness to rare and/or new candidate alleles was obtained.

TABLE 7
Accuracy vs. Depth of Coverage

| Coverage | | | | |
|---|---|---|---|---|
| Target | Samples | Calls | Match | Mismatch |
| 10 | 224 | 8 | 5 | 3 |
| 30 | 224 | 223 | 210 | 13 |
| 50 | 224 | 224 | 223 | 1 |
| 70 | 224 | 224 | 224 | 0 |
| 90 | 224 | 224 | 224 | 0 |
| 100 | 224 | 224 | 224 | 0 |
| 200 | 224 | 220 | 220 | 0 |

A method in accordance with the present disclosure was validated using cell lines with known genotypes. Further, the performance of the presently disclosed methods were compared with an alternative repeat calling software.

More specifically, the validation comprised sequencing 51 reference Coriell cell lines previously characterized by the CDC Get-RM project with orthogonally validated UGT1A1 repeat alleles. These cell lines comprise various known combinations of from 6 to 9 TA repeats, including different combinations of *1, *28, *36 and *37 genotypes (available on the Internet at cdc.gov/labquality/get-rm/index.html). See, e.g., Pratt et al., “Characterization of 137 Genomic DNA Reference Materials for 28 Pharmacogenetic Genes A GeT-RM Collaborative Project,” J Mol Diagnostics 18, 109-123 (2016).

Genotype calls obtained using the presently disclosed methods were compared with those made by the Expansion Hunter software. See, e.g., Dolzhenko et al., “ExpansionHunter: A sequence-graph based tool to analyze variation in short tandem repeat regions,” Bioinformatics 35, 4754-4756 (2019). Generally, as shown in Table 8, genotype calls obtained using the presently disclosed methods (“Bayesian”) matched the truth set with 100% accuracy, except for NA20509, where a SNV was also present in the repeat. Highly accurate genotype calling was observed for both homozygous (“Hom”) and heterozygous (“Het”) genotypes. By contrast, Expansion Hunter exhibited a significant error rate for the *28/*28 homozygotes.

TABLE 8
Performance with GET-RM Cell Lines

| | | | | Bayesian | | Expansion Hunter | |
|---|---|---|---|---|---|---|---|
| Genotype | Repeat | Zygosity | Cell Lines | Match | Mismatch | Match | Mismatch |
| *1/*1 | 7/7 | Hom | 15 | 15 | 0 | 15 | 0 |
| *1/*36 | 6/7 | Het | 4 | 4 | 0 | 4 | 0 |
| *1/*37 | 7/9 | Het | 2 | 2 | 0 | 2 | 0 |
| *1/*38 | 7/8 | Het | 15 | 15 | 0 | 15 | 0 |
| *28/*28 | 8/8 | Hom | 11 | 10 | 1 | 1 | 10 |
| *28/*36 | 6/8 | Het | 3 | 3 | 0 | 3 | 0 |
| *28/*37 | 8/9 | Het | 1 | 1 | 0 | 1 | 0 |

Additionally, similar performance was confirmed using 66 patient samples where true status was confirmed orthogonally by fragment analysis.

As evidenced by the foregoing, the methods and systems of the present disclosure allow for automated and accurate UGT1A1 promotor genotyping from targeted NGS data. In some embodiments, such methods are further applicable to other genomic repeat regions of clinical relevance. Particularly, the presently disclosed methods and systems identify UGT1A1 repeat polymorphisms associated with therapeutic agent-associated (e.g., IRI-induced) adverse events. In some implementations, such methods have utility in clinical NGS testing to further support clinician treatment decisions for cancer patients.

### Example 2—UGT1A1 Germline Caller to Screen Cancer Patients for Risk of Toxicity to Drugs

A UGT1A1 germline caller was developed to screen cancer patients for risk of toxicity to irinotecan, sacituzumab govitecan, and belinostat, based on repeat sequence polymorphisms in the TATA box of the UGT1A1 promoter region. In some implementations, the caller can be ordered by clinicians using a requisition form or in an online portal, and using previously obtained NGS sequencing data, without the need for additional tissue collection.

In some embodiments, the UGT1A1 caller is used in pan-cancer context. For instance, in some embodiments, the caller is used for colorectal cancer patients (e.g., for patients being considered for FOLFOX or FOLFIRI regimens) as well as for gastrointestinal and breast cancer patients. For instance, in some implementations, patients who receive irinotecan, trodelvy, and/or belinostat as a part of their drug regimen, are considered candidates for the UGT1A1 caller.

### SUMMARY

In some implementations, the clinical utility of the UGT1A1 algorithm is to identify patients who are at elevated risk for severe adverse events to Irinotecan, Trodelvy, and Belinostat who might benefit from increased monitoring or dose reduction. In some embodiments, the test considers patients who have the variant combinations *6/*6, *6/*28, or *28/*28 as “positive” and at high risk for toxicity from any one or more of the three drugs, in accordance with a January 2022 update to the Irinotecan drug label. Advantageously, this reporting strategy is more inclusive than many competitors including Quest and LabCorp who only report *28/*28, and provides meaningful risk information for patients in minority populations. In some implementations, other allele combinations found that do not amount to a positive report are included in a report comprising variant calls, including repeat sequence polymorphism.

In some embodiments, the UGT1A1 is applicable to a substantial proportion (e.g., at least 10%, at least 15%, at least 20%, at least 25%, at least 30%, at least 40%, at least 50%, or at least 60%) of subjects having a cancer condition. For example, in a sample population of tumor-normal matched patients, around 12% were expected to test positive for UGT1A1 deficiency, and hence were considered for monitoring or dose reduction. In various embodiments, monitoring or dose regimens for patients having abnormal UGT1A1 activity are selected according to established clinical guidelines for such patients. Example monitoring schedules and dose regimens recommended for various UGT1A1 polymorphisms are known in the art. For instance, various studies have reported maximum tolerated doses of irinotecan of 220 mg/m², 90 mg/m², or 75 mg/m² for the *28/*28 genotype, 150 mg/m² or 240 mg/m² for the *6 and *28 homozygous genotypes, and 210 mg/m² for the *28/*28 genotype. In some embodiments, recommended monitoring and dose regimens are based on one or more characteristics of the subject or a sample therefrom, such as cancer type, surgery status, metastatic status, histological features, ethnicity, and/or medication. Non-limiting examples of therapeutic regimens recommended for various genotypes are further described, for instance, in Argevani et al., “Dosage adjustment of irinotecan in patients with UGT1A1 polymorphisms: a review of current literature.” Innov Pharm. 2020; 11(3): 10.24926/iip.v11i3.3203; and Hulshof et al., “Pre-therapeutic UGT1A1 genotyping to reduce the risk of irinotecan-induced severe toxicity: Ready for prime time.” Eur J Cancer. 2020; 141: 9-20, each of which is hereby incorporated herein by reference in its entirety.

The science behind UGT1A1 is known in the art, and UGT1A1 testing is available from a variety of providers (e.g., LabCorp, Quest, ARUP, etc.). However, UGT1A1 testing is not commonly ordered, due to the logistics of ordering an additional test and collecting an additional blood sample. Advantageously, in some embodiments, the UGT1A1 caller is performed using NGS sequencing data, and does not require the collection of additional biological samples for testing.

In some embodiments, the UGT1A1 caller comprises a Bayesian model, in accordance with the methods and systems of the present disclosure. In another example, the UGT1A1 caller comprises a machine learning or deep learning caller that is based on a machine learning algorithm trained on example data (e.g., DeepVariant).

In some embodiments, the UGT1A1 caller accurately identifies and/or differentiates between various repeat sequence polymorphisms in UGT1A1 variants.

In some embodiments, the UGT1A1 caller further identifies one or more single-nucleotide variants (SNVs) and/or insertion deletion variants (indels).

In some embodiments, the UGT1A1 caller identifies at least 2, at least 3, at least 4, at least 5, at least 6, at least 7, at least 8, at least 9, or at least 10 UGT1A1 alleles associated with toxicity risk. In some embodiments, the UGT1A1 caller identifies no more than 20, no more than 10, no more than 8, or no more than 5 UGT1A1 alleles associated with toxicity risk.

Validation

A wet-lab validation plan was performed using targeted panel NGS sequencing on tumor-normal matched samples (hereinafter, “xT assay”). To test that UGT1A1 alterations were accurately called, reference samples and residual patient samples positive for known UGT1A1 alleles were tested using the xT assay, and at an orthogonal lab, according to the conditions set forth in Table 9. Genomic DNA was isolated from blood or saliva for analysis by next-generation sequencing (NGS), and the assay used normal tissue results from tumor-normal matched samples.

TABLE 9
Example Conditions for Validation Testing of
UGT1A1 Caller

| | Number of | |
|---|---|---|
| Experiment | Samples | Notes |
| Accuracy | 57 reference | xT sequencing on all |
| | samples from | samples and confirmatory |
| | GetRM repository | Sanger or PCR amplified |
| | or cell lines | capillary electrophoresis. |
| Sensitivity + | 79 samples | Sanger or PCR amplified |
| Specificity | | capillary electrophoresis |
| | | must be done to show algo |
| | | concordance with reference |
| | | lab calling for 5 alleles |
| Intra-Assay | 9 samples | 3 samples, in triplicate |
| Precision | | |
| Inter-Assay | 9 samples | 3 samples, in triplicate |
| Precision | | |
| Limit of | 15 samples | 5 DNA input levels into |
| Detection | (×5 levels) | library preparation ranging |
| | | from 25 ng to 600 ngx |
| Interfering | 4 samples | Ethanol and 2 concentrations |
| Substances | (×3 interferents) | of hemoglobin from blood |
| | | and salvia |
| Intersequencer | 13 samples | 13 samples sequenced across |
| concordance | | at least 3 NovaSeq 6000 |
| | | instruments. |

The results of the validation assay are provided in further detail in Example 3, below (Example 3: Validation of UGT1A1 Polymorphism Genotyping).

Reporting

In some embodiments, the UGT1A1 caller provides three different types of reports. These include “positive,” “normal,” and “see summary of findings” reports. The positive report is for patients with either *6/*6, *28/*28, or *6/*28. The normal report is for patients who have alleles associated with standard toxicity risk. The summary of findings report is for patients with variants that are not normal, but have insufficient clinical evidence to show they are at risk for toxicity events.

In some implementations, candidate alleles for the UGT1A1 repeat sequence that are called by the UGT1A1 caller include wild-type UGT1A1, containing six TA repeats [A(TA)₆TAA] in its promoter region (also known as the *1 allele); polymorphic UGT1A1 alleles with a higher number of TA repeats, such as UGT1A1*28/(TA)₇ and *37/(TA)₈ alleles, which cause decreased enzyme activity and are associated with severe toxicity in patients receiving IRI-based chemotherapy; and a polymorphic UGT1A1 allele with a lower number of TA repeats known as UGT1A1*36/(TA)₅, which has enzyme activity that is greater than or equal to normal limits (e.g., wild-type activity).

In some embodiments, additional variants that are called by the UGT1A1 caller include alleles comprising SNVs such as *6 (Gly71Arg) and *27 (Pro229Glu).

In some implementations, the reported genetic alterations, metabolism status, and/or toxicity risk are defined based on drug labeling and guidelines published by the Pharmacogenomics Knowledgebase (PharmGKB) knowledge base (PMID: 34216021), and/or any literature databases known in the art.

As described above, in some implementations, a positive test result is reported when a genotype for the genomic locus comprising the repeat sequence is homozygous *28 (*28/*28), homozygous *6 (*6/*6), or compound heterozygous *6 and *28 (*6/*28). In some such embodiments, the genotype is considered clinically actionable in accordance with relevant drug labeling and/or the PharmGKB knowledge base.

In some implementations, a normal test result is reported when a genotype for the genomic locus comprising the repeat sequence is *1/*1, *1/*36, or *36/*36, each of which is anticipated to have enzyme activity being greater than or equal to reference activity (e.g., normal limits).

In some implementations, a “See Summary of Findings” result is reported when alterations not associated with a positive or normal result are identified. Specifically, in some such embodiments, a “See Summary of Findings” result is reported when the caller identifies the UGT1A1 variants *27 and *37, which are associated with decreased enzyme activity in accordance with the PharmGKB knowledge base.

UGT1A1 alterations *6, *27, *28, and *37 are predicted to result in decreased enzyme activity and are associated with Gilbert syndrome, which is a benign autosomal recessive disorder of bilirubin metabolism (benign familial hyperbilirubinemia). Diagnosis of Gilbert syndrome is based on the level of unconjugated bilirubin in the blood and DNA variants. Non genetic factors can impact bilirubin levels. Accordingly, in some embodiments, the report includes one or more UGT1A1 genotype associations with Gilbert syndrome. In some embodiments, the report provides a recommendation for additional confirmatory testing of Gilbert syndrome.

Example reports are illustrated in FIGS. 11A-C, in accordance with the methods and systems of the present disclosure.

FIG. 11A provides an illustrative example of a “Positive” report. In some implementations, a “Positive” report includes a header 1102 comprising one or more of a patient name (e.g., “Joe Smith”), an accession number (e.g., “Accession No. TL-22-RDD4F8JB”), and a genomic locus name (e.g., “UGT1A1”). In some implementations, the “Positive” report further includes a report metadata section 1104, including one or more of patient metadata (e.g., “Date of Birth,” “Sex”), report metadata (e.g., “Physician,” “Institution,” “Provider,” “UGT1A1”), and sample metadata (e.g., “Specimen,” “Collected on,” “Received on”). In some implementations, the “Positive” report further includes a report overview 1106, including one or more of a title (e.g., “UDP GLUCURONOSYLTRANSFERASE FAMILY 1 MEMBER A1 VARIANT RESULT”), and a report type (e.g., “Positive: Patient is a poor metabolizer”). In some embodiments, the “Positive” report includes a summary of findings 1108 that provides one or more of an overview of the determined genotype for the genomic locus comprising the repeat sequence (e.g., “*6/*6”), one or more drug recommendations, and one or more clinical or research based recommendations. For example, the example summary of findings 1108 illustrated in FIG. 11A includes the description: “Summary of Findings: *6/*6 was detected in this patient. This genotype is expected to result in decreased UGT1A1 enzyme activity (poor metabolizer). The following drug is metabolized by UGT1A1: Irinotecan. Therefore the patient is at elevated risk for toxicity from this drug. See drug labeling (Irinotecan, Irinotecal Liposome Injection) and/or PharmGKB for dosing recommendations and clinical annotation. This genotype has been associated with the diagnosis of Gilbert syndrome. Gilbert syndrome is a common benign autosomal recessive disorder characterized by elevated levels of bilirubin in the blood. Clinical correlation and monitoring are recommended. See References Table for more information.” In some embodiments, the “Positive” report includes an assay interpretation 1110 that provides one or more of an overview of the determined genotype information presented in the report and an overview of a method for determining a result using the determined genotype information. For example, the example assay interpretation 1110 illustrated in FIG. 11A includes the description: “PROVIDER considers a positive test result to be a finding of one of the three genotypes considered clinically actionable in accordance with PharmGKB. These three results are homozygous *28 (*28/*28), homozygous *6 (*6/*6), or compound heterozygous *6 and *28 (*6/*28). PROVIDER considers a normal result to be when the patient is within normal enzyme limits with genotypes *1/*1, *1/*36, or *36/*36 in accordance with PharmGKB. This test is validated to report additional biologically relevant UGT1A1 variants and will display them in the variant table and provide relevant information in the Summary of Findings. These biologically relevant variants have insufficient evidence to support dosing changes. See Assay Description for more information.” In some embodiments, the “Positive” report further includes a genomic locus summary 1112, including a variant name (e.g., “Variant”), a repeat count for a number of repeat units in a tandem repeat in the genomic locus (e.g., “Repeat”), a variant identifier (e.g., “HGVS”), a zygosity (e.g., “homozygote” or “heterozygote”), and/or a result (e.g., “decreased function” or “normal function”).

FIG. 11B provides an illustrative example of a “Normal” report. In some implementations, a “Normal” report includes a header 1102 comprising one or more of a patient name (e.g., “Hunter Tremblay”), an accession number (e.g., “Accession No. TL-22-RDD4F8JB”), and a genomic locus name (e.g., “UGT1A1”). In some implementations, the “Normal” report further includes a report metadata section 1104, including one or more of patient metadata (e.g., “Date of Birth,” “Sex”), report metadata (e.g., “Physician,” “Institution,” “Provider,” “UGT1A1”), and sample metadata (e.g., “Specimen,” “Collected on,” “Received on”). In some implementations, the “Normal” report further includes a report overview 1106, including one or more of a title (e.g., “UDP GLUCURONOSYLTRANSFERASE FAMILY 1 MEMBER A1 VARIANT RESULT”), and a report type (e.g., “Normal”). In some embodiments, the “Normal” report includes a summary of findings 1108 that provides one or more of an overview of the determined genotype for the genomic locus comprising the repeat sequence (e.g., “*36/*36”), one or more drug recommendations, and one or more clinical or research based recommendations. For example, the example summary of findings 1108 illustrated in FIG. 11B includes the description: “Summary of Findings: *36/*36 was detected in this patient. This patient is expected to have UGT1A1 enzyme activity within normal limits. See References Table for more information.” In some embodiments, the “Normal” report includes an assay interpretation 1110 that provides one or more of an overview of the determined genotype information presented in the report and an overview of a method for determining a result using the determined genotype information. For example, the example assay interpretation 1110 illustrated in FIG. 11B includes the description: “PROVIDER considers a positive test result to be a finding of one of the three genotypes considered clinically actionable in accordance with PharmGKB. These three results are homozygous *28 (*28/*28), homozygous *6 (*6/*6), or compound heterozygous *6 and *28 (*6/*28). PROVIDER considers a normal result to be when the patient is within normal enzyme limits with genotypes *1/*1, *1/*36, or *36/*36 in accordance with PharmGKB. This test is validated to report additional biologically relevant UGT1A1 variants and will display them in the variant table and provide relevant information in the Summary of Findings. These biologically relevant variants have insufficient evidence to support dosing changes. See Assay Description for more information.” In some embodiments, the “Normal” report further includes a genomic locus summary 1112, including a variant name (e.g., “Variant”), a repeat count for a number of repeat units in a tandem repeat in the genomic locus (e.g., “Repeat”), a variant identifier (e.g., “HGVS”), a zygosity (e.g., “homozygote” or “heterozygote”), and/or a result (e.g., “decreased function” or “normal function”).

FIG. 11C provides an illustrative example of a “See Summary of Findings” report. In some implementations, a “See Summary of Findings” report includes a header 1102 comprising one or more of a patient name (e.g., “Hunter Tremblay”), an accession number (e.g., “Accession No. TL-22-RDD4F8JB”), and a genomic locus name (e.g., “UGT1A1”). In some implementations, the “See Summary of Findings” report further includes a report metadata section 1104, including one or more of patient metadata (e.g., “Date of Birth,” “Sex”), report metadata (e.g., “Physician,” “Institution,” “Provider,” “UGT1A1”), and sample metadata (e.g., “Specimen,” “Collected on,” “Received on”). In some implementations, the “See Summary of Findings” report further includes a report overview 1106, including one or more of a title (e.g., “UDP GLUCURONOSYLTRANSFERASE FAMILY 1 MEMBER A1 VARIANT RESULT”), and a report type (e.g., “See Summary of Findings”). In some embodiments, the “See Summary of Findings” report includes a summary of findings 1108 that provides one or more of an overview of the determined genotype for the genomic locus comprising the repeat sequence (e.g., “*27/*37/*37”), one or more drug recommendations, and one or more clinical or research based recommendations. For example, the example summary of findings 1108 illustrated in FIG. 11C includes the description: “Summary of Findings: *27/*37/*37 was detected in this patient. This genotype is expected to result in decreased UGT1A1 enzyme activity (poor metabolizer). There is insufficient evidence to recommend dosing changes. This genotype has been associated with the diagnosis of Gilbert syndrome. Gilbert syndrome is a common benign autosomal recessive disorder characterized by elevated levels of bilirubin in the blood. Clinical correlation and monitoring are recommended. See References Table for more information.” In some embodiments, the “See Summary of Findings” report includes an assay interpretation 1110 that provides one or more of an overview of the determined genotype information presented in the report and an overview of a method for determining a result using the determined genotype information. For example, the example assay interpretation 1110 illustrated in FIG. 11C includes the description: “PROVIDER considers a positive test result to be a finding of one of the three genotypes considered clinically actionable in accordance with PharmGKB. These three results are homozygous *28 (*28/*28), homozygous *6 (*6/*6), or compound heterozygous *6 and *28 (*6/*28). PROVIDER considers a normal result to be when the patient is within normal enzyme limits with genotypes *1/*1, *1/*36, or *36/*36 in accordance with PharmGKB. This test is validated to report additional biologically relevant UGT1A1 variants and will display them in the variant table and provide relevant information in the Summary of Findings. These biologically relevant variants have insufficient evidence to support dosing changes. See Assay Description for more information.” In some embodiments, the “See Summary of Findings” report further includes a genomic locus summary 1112, including a variant name (e.g., “Variant”), a repeat count for a number of repeat units in a tandem repeat in the genomic locus (e.g., “Repeat”), a variant identifier (e.g., “HGVS”), a zygosity (e.g., “homozygote” or “heterozygote”), and/or a result (e.g., “decreased function” or “normal function”).

### Example 3—Validation of UGT1A1 Polymorphism Genotyping

Validation assays were performed to establish acceptability criteria and validate a method of determining genotypes for genomic variants in the UGT1A1 gene, including a genomic locus comprising a tandem repeat, in accordance with the systems and methods of the present disclosure.

For validation, UGT1A1 variant genotype determination (hereinafter, the “UGT1A1 test”) was performed for a plurality of variant sites including a genomic locus comprising a tandem repeat using a plurality of sequence reads obtained from the xT assay, and in accordance with some embodiments of the present disclosure. The UGT1A1 genotype determination validated the reference allele *1 and the positive alleles *6, *27, *28, *36, and *37 (see Table 10). The reported genetic alterations, metabolism status, and toxicity risk are defined based on authoritative sources such as the CPIC, FDA medication labels and FDA guidance (see, e.g., Table 11: Example References, below).

TABLE 10
Targeted Allele Summary.

| | Allele | | TA | Variant |
|---|---|---|---|---|
| Allele | Status | HG19 | Repeat | Type |
| *1 | Normal | 2:234668881_234668882: | TA6 | reference |
| | | TA[7] | | |
| *6 | Decreased | 2:234669144:G:A | N/A | SNV |
| | Function | | | |
| *27 | Decreased | 2:234669619:C:A | N/A | SNV |
| | Function | | | |
| *28 | Decreased | 2:234668881_234668882: | TA7 | indel |
| | expression | TA[8] | | |
| *36 | Increased | 2:234668881_234668882: | TA5 | indel |
| | expression | TA-[6] | | |
| *37 | Decreased | 2:234668881_234668882: | TA8 | indel |
| | expression | TA[9] | | |

TABLE 11
Example References

| Title | Description |
|---|---|
| FDA label | FDA label for Irinotecan, available on the |
| for Irinotecan | Internet at accessdata.fda.gov/drugsatfda_docs/ |
| | label/2014/020571s0481bl.pdf |
| FDA label | FDA label for Trodelvy, available on the |
| for Trodelvy | Internet at trodelvy. com/?gclid= |
| | CjwKCAiAm7OMBhAQEiwArvGi3CssmG- |
| | GDuJEoi5ECNdDC9ZQ8STfwrjtoqiIJroy8oUQ |
| | KJ17GsxchBoCvHYQAvD_BwE&gclsrc=aw.ds |
| CPIC | Clinical Pharmacogenetics Implementation |
| | Consortium (CPIC) Guideline for UGT1A1 and |
| | Atazanavir Prescribing Clin Pharmacol Ther |
| | 2016 Apr; 99(4):363-9. doi: 10.1002/cpt.269. |
| | Epub 2015 Nov 9. PMID: 26417955 |
| GeT-RM | Characterization of 137 Genomic DNA Reference |
| | Materials for 28 Pharmacogenetic Genes: A |
| | GeT-RM Collaborative Project. J Mol Diagn. |
| | (2016) Jan; 18(1):109-23. doi: 10.1016/j.jmoldx. |
| | 2015.08.005. PMID: 26621101. |
| PharmGKB | PharmGKB Resource, available on the Internet at |
| Resource | pharmgkb.org/page/ugt1a1RefMaterials |

See, e.g., FDA label for Irinotecan, available on the Internet at accessdatafda.gov/drugsatfda_docs/label/2014/020571s048lbl.pdf; FDA label for Trodelvy, available on the Internet at trodelvy.com/?gclid=CjwKCAiAm7OMBhAQEiwArvGi3CssmG-GDuJEoi5ECNdDC9ZQ8STfwrjtoqillroy8oUQKJ17GsxchBoCvHYQAvD_BwE&gclsrc=a w.ds; Clinical Pharmacogenetics Implementation Consortium (CPIC) Guideline for UGT1A1 and Atazanavir Prescribing Clin Pharmacol Ther 2016 April; 99(4):363-9. doi: 10.1002/cpt.269. Epub 2015 Nov. 9. PMID: 26417955; Characterization of 137 Genomic DNA Reference Materials for 28 Pharmacogenetic Genes: A GeT-RM Collaborative Project. J Mol Diagn. (2016) January; 18(1):109-23. doi: 10.1016/j.jmoldx.2015.08.005. PMID: 26621101; and PharmGKB Resource, available on the Internet at pharmgkb.org/page/ugt1a1RefMaterials, each of which is incorporated herein by reference in its entirety.

### Definitions

As used in this Example, the term “Germline Variation” refers to an inherited variation present in a patient's DNA.

As used in this Example, the term “Variant Allele Fraction (VAF)” refers to the percentage of sequence reads observed matching a specific DNA variant divided by the overall coverage at that locus, calculated as: (reads passing filter supporting the variant)/(total reads passing filter). In some embodiments, VAF is expressed as a fraction or as a percentage (if multiplied by 100).

As used in this Example, the term “Limit of Detection (LOD)” refers to the minimum analyte input that the assay can reliably detect. In some embodiments, the metric used to determine LOD varies (e.g., mass input, variant allele frequency, tumor purity, and/or cellular content) depending on features relevant to the specific analyte classification.

As used in this Example, the term “Single Nucleotide Variant (SNV)” refers to a variant where the sample differs from the reference sequence via a substitution at a single DNA base.

As used in this Example, the term “Insertion-Deletion Variant (INDEL)” refers to a variant where the sample differs from the reference sequence via insertion and/or deletion of greater than 1 nucleotide.

As used in this Example, the term “Positive Percent Agreement (PPA)” refers to the proportion of comparative/reference method positive results in which the test method result is positive. In some implementations, this is calculated as 100×(True positives)/(True positives+False negatives). For this Example, PPA is calculated as a point estimate unless otherwise noted.

As used in this Example, the term “Negative Percent Agreement (NPA)” refers to the proportion of comparative or reference method negative results in which the test method result is negative, calculated as 100×(True negatives)/(True negatives+False positives). For this example, NPA is calculated as a point estimate unless otherwise noted.

As used in this Example, the term “Overall Percent Agreement (OPA)” refers to the proportion of total results where the test method and the comparative method agree, calculated as 100×(True positives+True negatives)/total number of samples. In this Example, OPA is calculated as a point estimate unless otherwise noted.

As used in this Example, the term “False Negative (FN)” refers to the number of subjects or specimens with a negative test result for a subject in whom the condition of interest is present (as determined by the designated reference standard).

As used in this Example, the term “False Positive (FP)” refers to the number of subjects or specimens with a positive test result for a subject in whom the condition of interest is absent (as determined by the designated reference standard).

As used in this Example, the term “True Positive (TP)” refers to the number of subjects or specimens with a positive test result for a subject in whom the condition of interest is present (as determined by the designated reference standard).

As used in this Example, the term “True Negative (TN)” refers to the number of subjects or specimens with a negative test result for a subject in whom the condition of interest is absent (as determined by the designated reference standard).

As used in this Example, the term “Orthogonal Testing” refers to testing used for validation purposes performed by an outside College of American Pathologists (CAP)/Clinical Laboratory Improvement Amendments (CLIA) laboratory.

As used in this Example, the term “UGT1A1” refers to the gene encoding UDP glucuronosyltransferase family 1 member A1.

As used in this Example, the term “Normal” refers to a non-tumor sample used to measure the germline status of the subject.

As used in this Example, the term “SOP” refers to Standard Operating Procedure.

As used in this Example, the term “STRP” refers to a PCR amplified capillary electrophoresis or Short-tandem repeat polymorphism analysis.

Validation Summaries

A total of 154 unique samples consisting of DNA extracted from the GetRM cell line repository and clinical saliva and blood as detailed in Table 12 were used in this validation. Samples were sequenced on the Illumina NovaSeq 6000 using the xT assay, and genotypes for variant sites (e.g., including the genomic locus of the UGT1A1 gene comprising the tandem repeat) were determined using the UGT1A1 test. The orthogonal method used for confirmation of variant genotypes was either Sanger sequencing for SNVs or PCR amplified capillary electrophoresis (referred to herein as “short tandem repeat polymorphism (STRP)”) analysis for indels performed by an orthogonal CAP/CLIA certified lab. GetRM cell lines have characterized genotypes.

TABLE 12
Validation Sample Summary

| | | Samples Sent for |
|---|---|---|
| | Total | Orthogonal |
| Sample Description | Samples | Genotype Testing |
| Extracted DNA from Blood | 82 | 58 |
| Extracted DNA from Saliva | 23 | 20 |
| DNA from GetRM /Cell line | 57 | 6 |
| Total Unique Samples | 162 | 84 |

The validation data were collected across 13 sequencing runs on 8 different days. Run descriptions, flow cell information, and sequencer information are listed in Table 13.

TABLE 13
Validation Run Summary

| Run | Analysis | | |
|---|---|---|---|
| # | Description | Flow Cell ID | Instrument |
| 1 | Inter-instrument | HJLNYDSX2 | Nova02B |
| | concordance | | |
| 2 | Inter-instrument | HKLMNDSX2 | Nova02A |
| | concordance | | |
| 3 | Inter-instrument | HKMFHDSX2 | Nova06B |
| | concordance | | |
| 4 | Inter-Precision | HFMCJDSX3 | Nova08 |
| 5 | Inter-Precision | HNLG5DMXX | Nova03 |
| 6 | Inter-Precision; | HJWTHDSX2 | Nova04B |
| | Inter-instrument | | |
| | concordance; LOD | | |
| 7 | Inter-Precision; | HLL7YDSX2 | Nova05B |
| | Inter-instrument | | |
| | concordance; | | |
| | LOD; Interfering | | |
| | substance | | |
| 8 | Inter-Precision; | HKYWHDRXX | Nova05 |
| | Intra-Precision | | |
| 9 | Intra-Precision | HKYWHDRXX | Nova05 |
| 10 | Intra-Precision; | HH3LCDRXY, | Nova07A |
| | Inter-Precision; | HH3LMDRXY | |
| | Interfering | | |
| | substance | | |
| 11 | LOD | HK23JDRXY | Nova04A |
| 12 | LOD | HKCHWDRXY | Nova01B |
| 13 | LOD | HLL72DSX2 | Nova04B |

Limit of Detection and Mass Input Dynamic Range

DNA extracted from 14 clinical blood specimens and 1 clinical saliva specimen were titrated at the following input masses into library preparation: 25 ng, 50 ng, 100 ng, 300 ng, and 600 ng. Specimens were tested singularly at each input mass due to limited extracted nucleic acid availability to process specimens in triplicate. Samples that did not pass quality control (QC) metrics for the xT assay were excluded from analysis. Table 14 summarizes the results of QC testing and genotype determination for variant sites (e.g., including the genomic locus of the UGT1A1 gene comprising the tandem repeat), using the UGT1A1 test.

TABLE 14
Limit of Detection and Dynamic Range
Data Summary

| | Number | Number of Samples | |
|---|---|---|---|
| Concentration | of | Passing QC | OPA |
| (ng) | Samples | Criteria/Tested | (%) |
| 25 | 15 | 15 | 100 |
| 50 | 15 | 15 | 100 |
| 100 | 15 | 15 | 100 |
| 300 | 15 | 15 | 100 |
| 600 | 15 | 14* | 100 |

*A single sample library at 600 ng failed QC2 and was omitted from NGS data analysis.

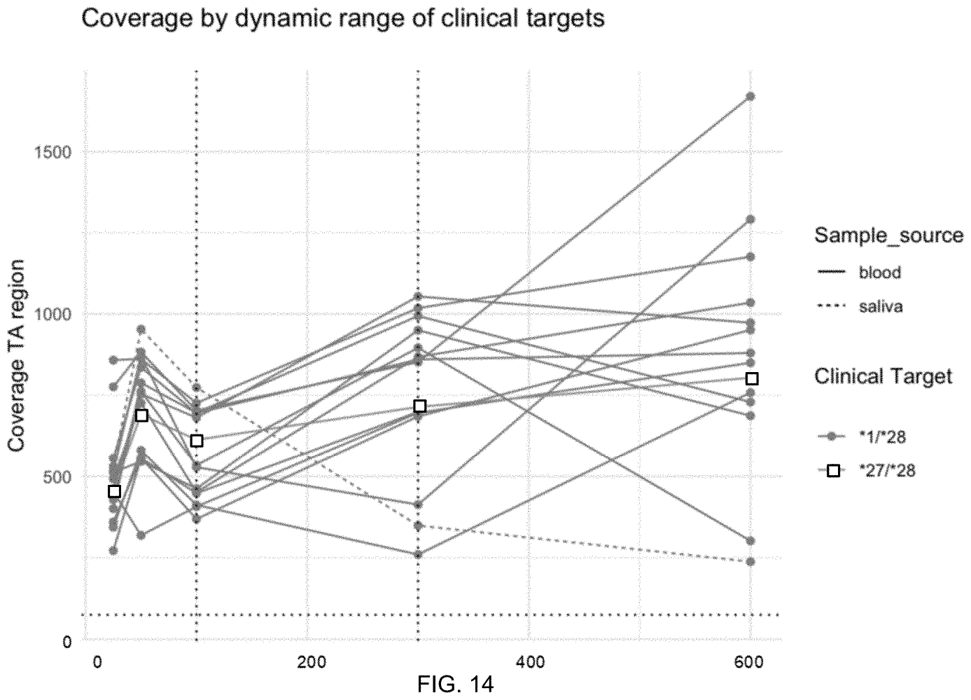

The sample mass input range was determined as 25 ng to 600 ng DNA input and confirmed to have no marked difference from the source material. All samples had sufficient coverage beyond 75×, as illustrated in FIG. 14. Moreover, the *27 heterozygous alterations at different concentrations had coverage of greater than 1400× (not shown). Notably, coverage for all titrations was shown to be sufficient within the validated input concentration range for the xT assay, which was defined as from 100 ng to 300 ng (shown in FIG. 14 as dotted vertical lines).

For each concentration, accuracy of genotype detection was evaluated as overall percent agreement (OPA), for sample titrations that met the acceptance criteria. Overall, the results summarized in Table 14 illustrate that the systems and methods for UGT1A1 genotype determination disclosed herein resulted in high accuracy genotype detection across the allowable input range.

Analytical Accuracy to Reference Specimens

57 total specimens were selected from the Get-RM repository or from the Coriell database (see, e.g., PMID: 26621101, provided in Table 11: Example References) based on their reported diplotype. A combination of heterozygous and homozygous alleles at the clinical sites of variation (e.g., *36, *37, *28, *27, *6, and *1/negative) were tested to evaluate the accuracy of genotype determination using the UGT1A1 test. All specimens were sequenced with the xT assay, with 6 samples being sent for orthogonal confirmation by STRP. The *1 targeted allele was considered a negative for this analysis and samples were evaluated at all targeted positions. Results of the UGT1A1 test and various metrics are summarized in Table 15.

TABLE 15
Analytical Accuracy to Reference by Targeted Allele Data Summary

| Targeted | # Samples | # Sample | # | # | | | | PPA | NPA |
|---|---|---|---|---|---|---|---|---|---|
| Allele | with Target | Failure** | Het | Horn | STRP | Concordant | Discordant | (%) | (%) |
| *36 | 7 | 0 | 7 | 0 | 3 | 7 | 0 | 100 | 100 |
| *37 | 5 | 0 | 5 | 0 | 2 | 5 | 0 | 100 | 100 |
| *28 | 32 | 6 | 21 | 5 | 1 | 31 | 0 | 100 | 100 |
| *27 | 1 | 0 | 1 | 0 | 0 | 1 | 0 | 100 | 100 |
| *6 | 5 | 1 | 3 | 1 | 0 | 5 | 0 | 100 | 100 |
| Negative | 12 | 0 | 0 | 12 | 0 | 72 | 0 | 100 | 100 |
| (*1) | | | | | | | | | |

In Table 15, column headings are indicated as follows: “Targeted Allele” denotes the genotype of the variant at the targeted position; “#Samples with Target” denotes the number of samples having the respective targeted allele; “#Sample Failure” denotes the number of samples excluded due to failure (described in further detail below); “#Het” denotes the number of heterozygous samples; “#Hom” denotes the number of homozygous samples; “STRP” denotes the total number of alleles, for the targeted allele, confirmed via STRP; “Concordant” denotes the total count of alleles having a concordance between the genotype determined using the UGT1A1 test and the known haplotype (e.g., as reported by the Get-RM repository or the Coriell database); “Discordant” denotes the total count of alleles having a discordance between the genotype determined using the UGT1A1 test and the known haplotype (e.g., as reported by the Get-RM repository or the Coriell database); “PPA %” denotes positive percent agreement; and “NPA %” denotes negative percent agreement.

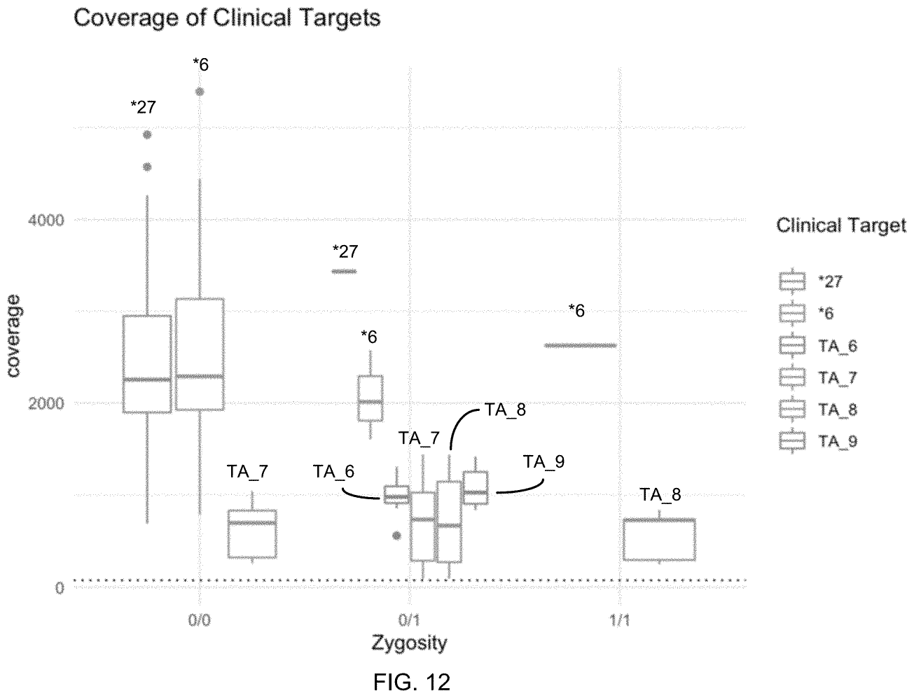

For all variant sites, sufficient coverage was achieved in the sequencing assay over validated thresholds (e.g., SNPs 20×; indels 75×), as shown in FIG. 12. Moreover, 49 positive and 72 negative alleles observed using the xT assay and genotyped using the UGT1A1 test were successfully matched to their known allele from the Get-RM repository or from the Coriell database.

Overall, as presented in Table 15, 100% NPA and PPA was observed for both SNP and indel alleles, illustrating that the UGT1A1 test demonstrated high analytical accuracy to gold standard reference specimens (e.g., Get-RM and/or Coriell databases).

One specimen in the plurality of 57 specimens (specimen NA20509) was discordant with the GetRM expected value (homozygous *28) and further analysis was performed. This sample was sequenced twice on the xT assay platform, and both times the results supported heterozygous *28. This sample was further sent for orthogonal validation using STRP which supported the heterozygous *28 call. A previously purchased specimen of this sample was validated, and visual inspection of the pileup confirmed that this sample contained the variant heterozygous *28. For this reason this sample was relabeled as heterozygous *28 and was set as concordant (see, e.g., Table 15). Another specimen in the plurality of 57 specimens (specimen NA19239) failed to match expected alleles, upon further analysis, was determined to be an incorrect sample. As such, this sample was removed from the analysis.

Analytical Accuracy to Clinical Specimens

79 total specimens previously sequenced with the xT sequencing panel were utilized for orthogonal verification by Sanger sequencing or STRP analysis. The *1 targeted allele was considered a negative for this assay and samples were evaluated at all targeted positions. A combination of heterozygotes and homozygotes were included as part of the targeted positions. Summary data for the UGT1A1 test result and various metrics are presented in Table 16.

For example, 7 specimens including 8 targeted alleles failed to pass QC and were excluded from the analysis. Of these, 4 failed to generate data by STRP analysis. 1 sample was indexed for the study but failed to meet the minimum sample quantity for STRP analysis (20-A63380) and was excluded. 2 samples (21-A23703 and 20-A92150) were excluded as they were outside the reportable range. 1 sample (20-A71068) had a discordant Sanger call (heterozygous *6) against the UGT1A1 test call (homozygous *1). This sample was resent for Sanger sequencing and was confirmed to correspond to the reference allele (*1). 213 alleles derived from 72 specimens were successfully analyzed by Sanger sequencing or STRP. Each sample had its zygosity evaluated for concordance between the xT assay and the orthogonal method.

TABLE 16
Analytical Accuracy to Clinical Specimens Targeted Allele Data Summary

| Targeted | # Samples | # Sample | # | # | | | PPA | NPA |
|---|---|---|---|---|---|---|---|---|
| Allele | with Target | Failure** | Het | Hom | STRP/Sanger | Discordant | (%) | (%) |
| *36 | 11 | 0 | 10 | 1 | 12 | 0 | 100 | 100 |
| *37 | 10 | 2 | 8 | 0 | 8 | 0 | 100 | 100 |
| *28 | 22 | 2 | 15 | 5 | 25 | 0 | 100 | 100 |
| *27 | 10 | 2 | 7 | 1 | 9 | 0 | 100 | 100 |
| *6 | 15 | 0 | 9 | 6 | 21 | 0 | 100 | 100 |
| Negative | 25 | 2 | 0 | 23 | 138 | 0 | 100 | 100 |
| (*1) | | | | | | | | |

In Table 16, column headings are indicated as follows: “Targeted Allele” denotes the genotype of the variant at the targeted position; “#Samples with Target” denotes the number of samples having the respective targeted allele; “#Sample Failure” denotes the number of samples excluded due to failure (described in further detail below); “#Het” denotes the number of heterozygous samples; “#Hom” denotes the number of homozygous samples; “STRP/Sanger” denotes the total number of alleles, for the targeted allele, confirmed via STRP or Sanger sequencing; “Discordant” denotes the total count of alleles having a discordance between the genotype determined using the UGT1A1 test and the known haplotype (e.g., as reported by the Get-RM repository or the Coriell database); “PPA %” denotes positive percent agreement; and “NPA %” denotes negative percent agreement.

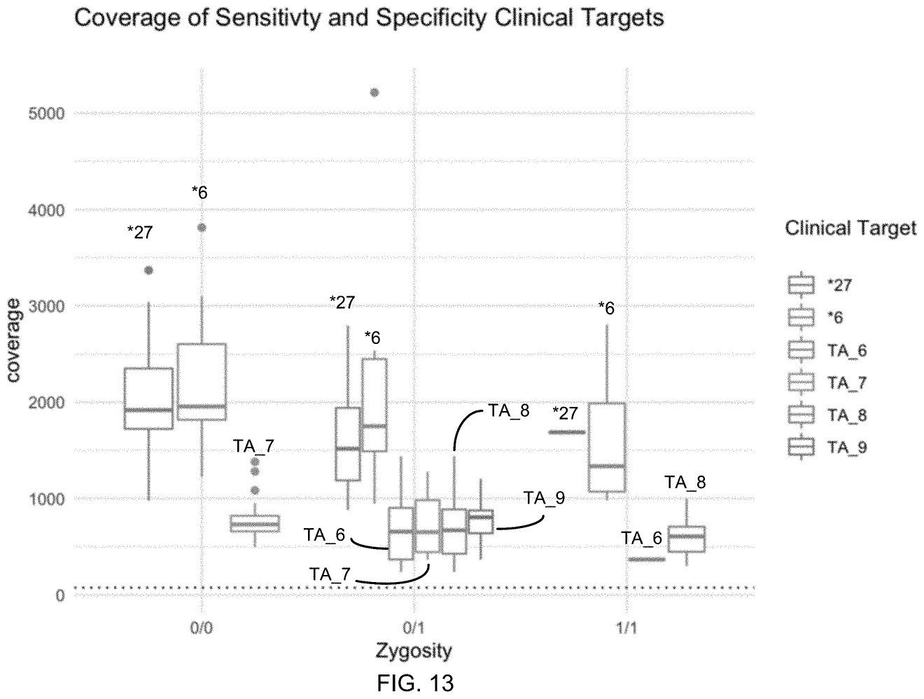

For all variant sites, sufficient coverage was achieved in the sequencing assay over validated thresholds (e.g., SNPs ≥20×; indels ≥75×), as shown in FIG. 13. Moreover, targeted allele and zygosity results showed 100% agreement between the UGT1A1 Tempus test and Sanger or STRP sequencing results. Additionally, 100% PPA and NPA for UGT1A1 alleles was observed relative to Sanger sequencing or STRP and thus all analytical accuracy acceptance criteria were met. Overall, these results demonstrate high analytical sensitivity and specificity of genotype determinations for clinical specimens using the UGT1A1 test.

Intra-Assay Precision

Three samples (1 SNV and 2 indel) were prepared in triplicate at 100 ng input using different barcodes within the same sequencing run. The data were analyzed by comparing the reference genotype between all samples within a group.

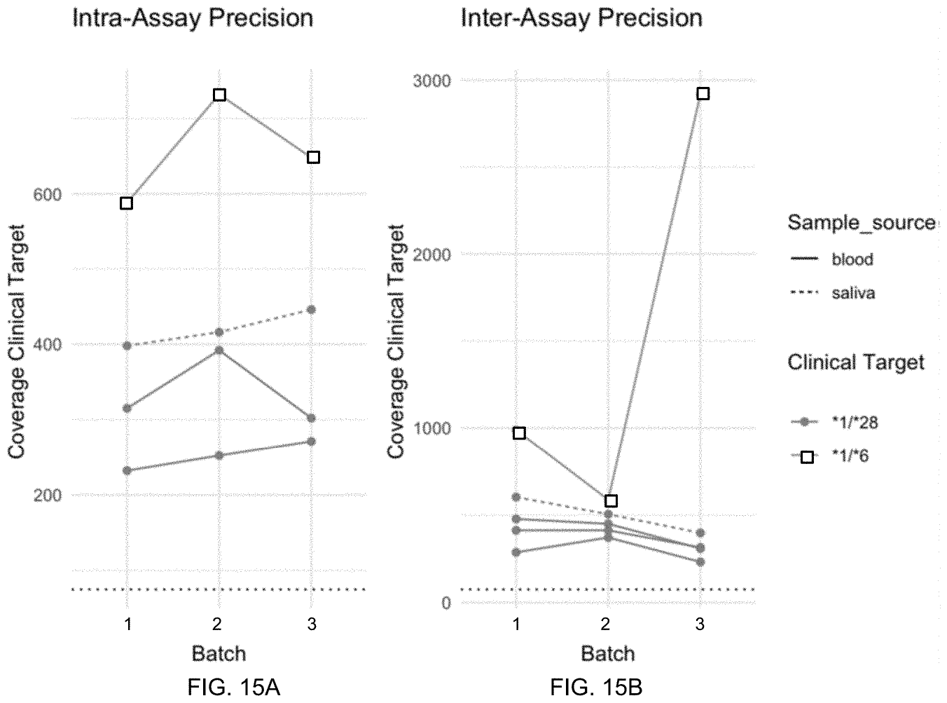

Acceptance criteria for the analysis included a threshold OPA of 100% based on genotype at minimum sample number, or ≥90% if 10 or more specimens were evaluated. All replicates passed QC, and OPA between replicates in this study was 100%. As shown in FIG. 15A, sufficient coverage over validated thresholds was achieved in the sequencing assay for all targeted variants.

Inter-Assay Precision

Five samples (1 SNV and 4 indel) were run at 1× repeat across 3 days for 3 total replicates each at 100 ng using different barcodes across the three separate runs on different days. The data were analyzed by comparing the reference genotype between all samples within a group.

Acceptance criteria for the analysis included a threshold OPA of 100% based on genotype at minimum sample number or ≥90% if 10 or more specimens were evaluated. All replicates passed QC, and OPA between replicates in this study was 100%. As shown in FIG. 15B, sufficient coverage over validated thresholds was achieved in the sequencing assay for all targeted variants.

Inter-Sequencer Concordance

13 samples were sequenced in triplicate across 3 different Illumina NovaSeq instruments. The samples included 10 blood samples and 3 saliva samples. Of these, 12 samples were run at a concentration of 100 ng DNA mass input and 1 sample was run at a concentration of 300 ng DNA mass input. Acceptance criteria for the analysis included a threshold OPA of ≥90% for detection of relevant genotype across multiple instruments.

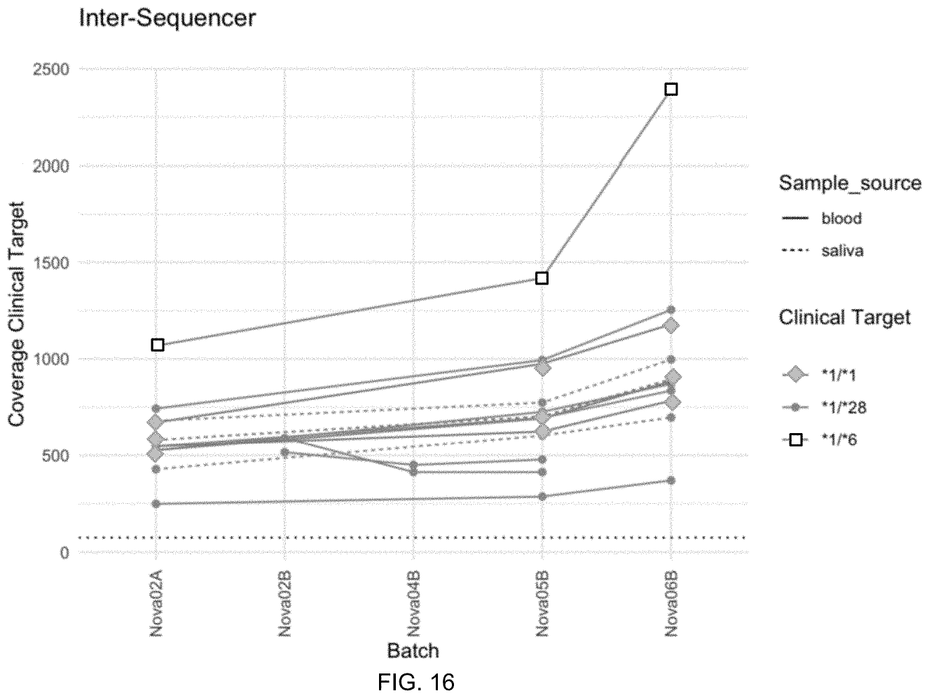

The 13 samples (over 39 replicates) were successfully sequenced across different instruments, demonstrating 100% OPA for genotype accuracy. As shown in FIG. 16, sufficient coverage over validated thresholds was achieved in the sequencing assay for all targeted variants and for both blood and saliva samples.

Interfering Substances

Hemoglobin was added at 1 g/L and 5 g/L into nucleic acid extracted from 4 blood specimens during normalization for library preparation. Ethanol was added at 10% into nucleic acid extracted from 3 blood specimens and 1 saliva specimen during normalization for library preparation. UGT1A1 test results from the contrived samples were compared to the non-contaminated reference samples for genotype agreement. Summary data for the UGT1A1 test results and various metrics are shown in Table 17.

TABLE 17
Interfering Substances Summary

| | | | Total | | | |
|---|---|---|---|---|---|---|
| | Sample | Total | Sample | Pass | | Zygosity |
| Substance | Source | Samples | Failure | Samples | OPA | OPA |
| Interf—Ethanol | Blood | 3 | 0 | 3 | 100% | 100% |
| Interf—Hb1 | Blood | 4 | 1 | 3 | 100% | 100% |
| Interf—Hb2 | Blood | 4 | 4 | 0 | 0% | 0% |
| Interf—Ethanol | Saliva | 1 | 0 | 1 | 100% | 100% |

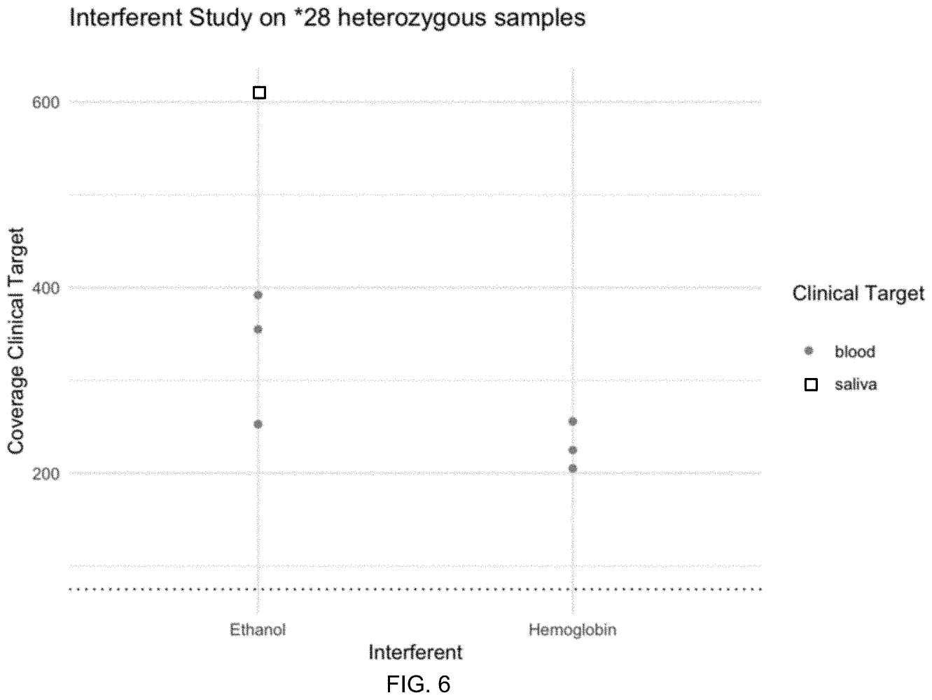

All 4 specimens tested with ethanol passed multiple QC tests (QC2-4) and were included in the analysis. All 4 specimens tested with 5 g/L hemoglobin and 1 specimen tested with 1 g/L hemoglobin failed at least one QC test (QC2) and were excluded from analysis. These results indicate that hemoglobin is likely to affect the generation of quality libraries. Acceptance criteria for the assays included, for each interfering substance, a threshold OPA of 100% for the genotype between non-interfering condition and interfering condition at the minimum sample number, or ≥90% if 10 or more samples were evaluated. As shown in FIG. 6, sufficient coverage over validated thresholds was achieved in the sequencing assay for all targeted variants and for both ethanol and hemoglobin samples.

For hemoglobin, all expected genotypes were called correctly, resulting in 100% OPA compared with reference sample results. For ethanol, all expected genotypes were called correctly, resulting in 100% OPA compared with reference sample results.

At the tested concentrations, ethanol was not identified as an interferant since it had no effect on the genotype calls. While a high rate of QC failure was observed under high hemoglobin concentrations, all replicates that passed all QC checks showed no impact on UGT1A1 results relative to non-contaminated reference samples. These results indicate that while, in some instances, hemoglobin interferes with the ability to produce viable cDNA libraries, it does not interfere with the ability of the assay to determine UGT1A1 genotype or zygosity status in successfully generated libraries. Therefore, in the case that excess hemoglobin is carried over from DNA extraction to library preparation at a high enough concentration to interfere with library preparation, quality control checks built into the device can be used to prevent the generation and reporting of results from any libraries with reduced analytical performance. The QC failures, and concordance observed for passing replicates, supports the validity of the QC metrics in assuring the quality of the assay. As a result, the acceptance criteria for the study were successfully met.

### CONCLUSION

In the above validation assays, all predetermined specifications and acceptance criteria for genotype calls were met, thus illustrating that the systems and methods of determining genotypes for genomic variants disclosed herein, such as the UGT1A1 test, is substantially controlled and reproducible. In particular, in some implementations, validated systems and methods of determining genotypes for genomic variants, such as those within a genomic locus comprising a tandem repeat (e.g., the UGT1A1 test), are acceptable for use in patient testing.

Summary of Samples Excluded During Data Analysis

Analytical Accuracy to Reference Specimens. Seven total samples were excluded. One specimen (NA19239) had greater overall variant divergence from a corresponding known Get-RM control indicating it was the incorrect sample and thus this sample was excluded from the analysis. 6 additional samples were excluded due to sample exhaustion before they could be verified by an orthogonal method (NA19921, NA17115, NA21133, NA10842, NA21130, NA20786).

Analytical Accuracy to Clinical Specimens. Seven total samples were excluded. Four positive specimens (20-A66829, 20-A75359, 22-A04282, 22-A07640) failed to produce STRP results. One sample (20-A63380) was excluded due to the specimen being exhausted before it could be verified by an orthogonal method. 2 samples (21-A23703 and 20-A92150) were excluded as they were outside the reportable range.

LOD and Mass Input Dynamic Range. One sample (21-A90648) failed a quality control step (QC2) for the 600 ng DNA mass input titration and was excluded from the analysis.

Interfering Substances. Five samples were excluded, including 4 samples (21-A90624, 21-A90648, 21-A90622, 21-A90620) of high hemoglobin (5 g/L hemoglobin added during normalization for library preparation) concentration and one sample (21-A90623) of low hemoglobin (1 g/L) that failed at a quality control step (QC2) during laboratory preparation.

End-to-End Testing

Using the validated UGT1A1 test, an end-to-end test was performed on 20 clinical cases with varying UGT1A1 alleles and metabolizer statuses to validate that results from a variant genotype determination and reporting pipeline were exactly concordant with expectations generated from the variant call and algorithm interpretation. Expected results were generated by pulling de-identified germline data from a reference database and compared to results obtained from the entire pipeline. Acceptance criteria for the analysis included a threshold of 100% agreement between PharmGKB and FDA interpretations compared with the reporting pipeline's calls for zygosity, Human Genome Variation Society (HGVS), metabolizer status, and result. All data generated in this analysis were 100% in agreement with expected results and the acceptance criteria were met. These results illustrate the accuracy and reliability of the presently disclosed systems and methods, and demonstrate their utility in a pipeline for clinical and/or research-based reporting and decision-making.

### Example 4—Digital and Laboratory Health Care Platform

In some embodiments, the methods and systems described above are utilized in combination with or as part of a digital and laboratory health care platform that is generally targeted to medical care and research. It should be understood that many uses of the methods and systems described above, in combination with such a platform, are possible. One example of such a platform is described in U.S. Patent Publication No. 2021/0090694, titled “Data Based Cancer Research and Treatment Systems and Methods,” and published Mar. 25, 2021, which is incorporated herein by reference and in its entirety for any and all purposes.

For example, an implementation of one or more embodiments of the methods and systems as described above includes microservices constituting a digital and laboratory health care platform supporting accurate genotyping of repeat polymorphisms from next-generation sequencing data. Certain embodiments include a single microservice for executing and delivering accurate genotyping of repeat polymorphisms or include a plurality of microservices each having a particular role which together implement one or more of the embodiments above. In some example embodiments, a first microservice executes realignment of repeat spanning reads to linear models of repeat expansion polymorphisms in order to deliver counts of spanning reads aligned to multiple linear models of the repeat expansion of varying lengths to a second microservice for Bayesian model analysis. Similarly, in some such embodiments, the second microservice executes Bayesian model analysis to produce posterior probabilities and to deliver final genotype calls with quality metrics according to an embodiment, above.

Where embodiments above are executed in one or more micro-services with or as part of a digital and laboratory health care platform, in some implementations, one or more of such micro-services are part of an order management system that orchestrates the sequence of events as needed at the appropriate time and in the appropriate order necessary to instantiate embodiments above. A micro-services based order management system is disclosed, for example, in U.S. Patent Publication No. 2020/80365232, titled “Adaptive Order Fulfillment and Tracking Methods and Systems,” and published Nov. 19, 2020, which is incorporated herein by reference and in its entirety for all purposes.

For example, continuing with the above first and second microservices, in some embodiments, an order management system notifies the first microservice that an order for accurate genotyping of repeat polymorphisms has been received and is ready for processing. In some such embodiments, the first microservice executes and notifies the order management system once the delivery of counts of spanning reads aligned to multiple linear models of the repeat expansion of varying lengths is ready for the second microservice. Furthermore, in some embodiments, the order management system identifies that execution parameters (prerequisites) for the second microservice are satisfied, including that the first microservice has completed, and notifies the second microservice that it may continue processing the order to deliver final genotype calls with quality metrics according to an embodiment, above.

Where the digital and laboratory health care platform further includes a genetic analyzer system, in some embodiments, the genetic analyzer system includes targeted panels and/or sequencing probes. An example of a targeted panel for sequencing cell-free (cf) DNA and determining various characteristics of a specimen based on the sequencing is disclosed, for example, in U.S. patent application Ser. No. 17/179,086, titled “Methods And Systems For Dynamic Variant Thresholding In A Liquid Biopsy Assay,” and filed Feb. 18, 1921, U.S. patent application Ser. No. 17/179,267, titled “Estimation Of Circulating Tumor Fraction Using Off-Target Reads Of Targeted-Panel Sequencing,” and filed Feb. 18, 1921, and U.S. patent application Ser. No. 17/179,279, titled “Methods And Systems For Refining Copy Number Variation In A Liquid Biopsy Assay,” and filed Feb. 18, 1921 which is incorporated herein by reference and in its entirety for all purposes. In one example, targeted panels enable the delivery of next-generation sequencing results (including sequencing of DNA and/or RNA from solid or cell-free specimens) for accurate genotyping of repeat polymorphisms according to an embodiment, above. An example of the design of next-generation sequencing probes is disclosed, for example, in U.S. Patent Publication No. 2021/0115511, titled “Systems and Methods for Next Generation Sequencing Uniform Probe Design,” and published Jun. 22, 2021, and U.S. patent application Ser. No. 17/323,986, titled “Systems and Methods for Next Generation Sequencing Uniform Probe Design,” and filed May 18, 1921, which are incorporated herein by reference and in their entirety for all purposes.

Where the digital and laboratory health care platform further includes an epigenetic analyzer system, in some embodiments, the epigenetic analyzer system analyzes specimens to determine their epigenetic characteristics and further uses that information for monitoring a patient over time. An example of an epigenetic analyzer system is disclosed, for example, in U.S. patent application Ser. No. 17/352,231, titled “Molecular Response And Progression Detection From Circulating Cell Free DNA,” and filed Jun. 18, 1921, which is incorporated herein by reference and in its entirety for all purposes.

Where the digital and laboratory health care platform further includes a bioinformatics pipeline, in some embodiments, the methods and systems described above are utilized after completion or substantial completion of the systems and methods utilized in the bioinformatics pipeline. As one example, the bioinformatics pipeline receives next-generation genetic sequencing results and returns a set of binary files, such as one or more BAM files, reflecting DNA and/or RNA read counts aligned to a reference genome. In some embodiments, the methods and systems described above are utilized, for example, to ingest the DNA and/or RNA read counts and produce accurate genotyping of repeat polymorphisms as a result.

When the digital and laboratory health care platform further includes an RNA data normalizer, in some embodiments, any RNA read counts are normalized before processing embodiments as described above. An example of an RNA data normalizer is disclosed, for example, in U.S. Patent Publication No. 2020/0098448, titled “Methods of Normalizing and Correcting RNA Expression Data,” and published Mar. 26, 2020, which is incorporated herein by reference and in its entirety for all purposes.

When the digital and laboratory health care platform further includes a genetic data deconvolver, in some embodiments, any system and method for deconvolving can be utilized for analyzing genetic data associated with a specimen having two or more biological components to determine the contribution of each component to the genetic data and/or determine what genetic data would be associated with any component of the specimen if it were purified. An example of a genetic data deconvolver is disclosed, for example, in U.S. Patent Publication No. 2020/0210852, published Jul. 2, 2020, and PCT/US19/69161, filed Dec. 31, 2019, both titled “Transcriptome Deconvolution of Metastatic Tissue Samples,” and U.S. patent application Ser. No. 17/074,984, titled “Calculating Cell-type RNA Profiles for Diagnosis and Treatment,” and filed Oct. 20, 2020, the contents of each of which are incorporated herein by reference and in their entirety for all purposes.

In some embodiments, RNA expression levels are adjusted to be expressed as a value relative to a reference expression level. Furthermore, in some embodiments, multiple RNA expression data sets are adjusted, prepared, and/or combined for analysis and/or are adjusted to avoid artifacts caused when the data sets have differences because they have not been generated by using the same methods, equipment, and/or reagents. An example of RNA data set adjustment, preparation, and/or combination is disclosed, for example, in U.S. patent application Ser. No. 17/405,025, titled “Systems and Methods for Homogenization of Disparate Datasets,” and filed Aug. 18, 2021.

When the digital and laboratory health care platform further includes an automated RNA expression caller, in some embodiments, RNA expression levels associated with multiple samples are compared to determine whether an artifact is causing anomalies in the data. An example of an automated RNA expression caller is disclosed, for example, in U.S. Pat. No. 11,043,283, titled “Systems and Methods for Automating RNA Expression Calls in a Cancer Prediction Pipeline,” and issued Jun. 22, 2021, which is incorporated herein by reference and in its entirety for all purposes.

In some embodiments, the digital and laboratory health care platform further includes one or more insight engines to deliver information, characteristics, or determinations related to a disease state that may be based on genetic and/or clinical data associated with a patient, specimen and/or organoid. In some embodiments, exemplary insight engines include a tumor of unknown origin (tumor origin) engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, a T cell receptor or B cell receptor profiling engine, a line of therapy engine, a metastatic prediction engine, and so forth.

An example tumor origin or tumor of unknown origin engine is disclosed, for example, in U.S. patent application Ser. No. 15/930,234, titled “Systems and Methods for Multi-Label Cancer Classification,” and filed May 12, 1920, which is incorporated herein by reference and in its entirety for all purposes.

An example of an HLA LOH engine is disclosed, for example, in U.S. Pat. No. 11,081,210, titled “Detection of Human Leukocyte Antigen Class I Loss of Heterozygosity in Solid Tumor Types by NGS DNA Sequencing,” and issued Aug. 3, 2021, which is incorporated herein by reference and in its entirety for all purposes.

An example of a tumor mutational burden (TMB) engine is disclosed, for example, in U.S. Patent Publication No. 2020/0258601, titled “Targeted-Panel Tumor Mutational Burden Calculation Systems and Methods,” and published Aug. 13, 2020, which is incorporated herein by reference and in its entirety for all purposes.

An example of a PD-L1 status engine is disclosed, for example, in U.S. Patent Publication No. 2020/0395097, titled “A Pan-Cancer Model to Predict The PD-L1 Status of a Cancer Cell Sample Using RNA Expression Data and Other Patient Data,” and published Dec. 17, 2020, which is incorporated herein by reference and in its entirety for all purposes. An additional example of a PD-L1 status engine is disclosed, for example, in U.S. Pat. No. 10,957,041, titled “Determining Biomarkers from Histopathology Slide Images,” issued Mar. 23, 2021, which is incorporated herein by reference and in its entirety for all purposes.

An example of a homologous recombination deficiency engine is disclosed, for example, in U.S. Pat. No. 10,975,445 and PCT/US20/18002, both titled “An Integrative Machine-Learning Framework to Predict Homologous Recombination Deficiency,” and filed Feb. 12, 1920, which is incorporated herein by reference and in its entirety for all purposes.

An example of a cellular pathway activation report engine is disclosed, for example, in U.S. Patent Publication No. 2021/0057042, titled “Systems And Methods For Detecting Cellular Pathway Dysregulation In Cancer Specimens,” and published Feb. 25, 2021, which is incorporated herein by reference and in its entirety for all purposes.

An example of an immune infiltration engine is disclosed, for example, in U.S. Patent Publication No. 2020/0075169, titled “A Multi-Modal Approach to Predicting Immune Infiltration Based on Integrated RNA Expression and Imaging Features,” and published Mar. 5, 2020, which is incorporated herein by reference and in its entirety for all purposes.

An example of an MSI engine is disclosed, for example, in U.S. Patent Publication No. 2020/0118644, titled “Microsatellite Instability Determination System and Related Methods,” and published Apr. 16, 2020, which is incorporated herein by reference and in its entirety for all purposes. An additional example of an MSI engine is disclosed, for example, in U.S. Patent Publication No. 2021/0098078, titled “Systems and Methods for Detecting Microsatellite Instability of a Cancer Using a Liquid Biopsy,” and published Apr. 1, 2021, which is incorporated herein by reference and in its entirety for all purposes.

An example of a pathogen infection status engine is disclosed, for example, in U.S. Pat. No. 11,043,304, titled “Systems And Methods For Using Sequencing Data For Pathogen Detection,” and issued Jun. 22, 2021, which is incorporated herein by reference and in its entirety for all purposes. Another example of a pathogen infection status engine is disclosed, for example, in PCT/US21/18619, titled “Systems And Methods For Detecting Viral DNA From Sequencing,” and filed Feb. 18, 2021, which is incorporated herein by reference and in its entirety for all purposes.

An example of a T cell receptor or B cell receptor profiling engine is disclosed, for example, in U.S. patent application Ser. No. 17/302,030, titled “TCR/BCR Profiling,” and filed Apr. 21, 2021, which is incorporated herein by reference and in its entirety for all purposes.

An example of a line of therapy engine is disclosed, for example, in U.S. Patent Publication No. 2021/0057071, titled “Unsupervised Learning And Prediction Of Lines Of Therapy From High-Dimensional Longitudinal Medications Data,” and published Feb. 25, 2021, which is incorporated herein by reference and in its entirety for all purposes.

An example of a metastatic prediction engine is disclosed, for example, in U.S. Pat. No. 11,145,416, titled “Predicting likelihood and site of metastasis from patient records,” and issued Oct. 12, 2021, which is incorporated herein by reference and in its entirety for all purposes.

When the digital and laboratory health care platform further includes a report generation engine, in some embodiments, the methods and systems described above are utilized to create a summary report of a patient's genetic profile and the results of one or more insight engines for presentation to a physician. For instance, in some embodiments, the report provides to the physician information about the extent to which the specimen that was sequenced contained tumor or normal tissue from a first organ, a second organ, a third organ, and so forth. For example, in some embodiments, the report provides a genetic profile for each of the tissue types, tumors, or organs in the specimen. In some embodiments, the genetic profile represents genetic sequences present in the tissue type, tumor, or organ and may include variants, expression levels, information about gene products, or other information that could be derived from genetic analysis of a tissue, tumor, or organ.

In some embodiments, the report includes therapies and/or clinical trials matched based on a portion or all of the genetic profile or insight engine findings and summaries. For example, in some embodiments, the clinical trials are matched according to the systems and methods disclosed in U.S. Patent Publication No. 2020/0381087, titled “Systems and Methods of Clinical Trial Evaluation,” published Dec. 3, 2020, which is incorporated herein by reference and in its entirety for all purposes.

In some embodiments, the report includes a comparison of the results (for example, molecular and/or clinical patient data) to a database of results from many specimens. An example of methods and systems for comparing results to a database of results are disclosed in U.S. Patent Publication No. 2020/0135303 titled “User Interface, System, And Method For Cohort Analysis” and published Apr. 30, 2020, and U.S. Patent Publication No. 2020/0211716 titled “A Method and Process for Predicting and Analyzing Patient Cohort Response, Progression and Survival,” and published Jul. 2, 2020, which is incorporated herein by reference and in its entirety for all purposes. In some embodiments, the information is used, sometimes in conjunction with similar information from additional specimens and/or clinical response information, to match therapies likely to be successful in treating a patient, discover biomarkers or design a clinical trial.

In some embodiments, any data generated by the systems and methods and/or the digital and laboratory health care platform is downloadable by the user. In one example, the data is downloaded as a CSV file comprising clinical and/or molecular data associated with tests, data structuring, and/or other services ordered by the user. In various embodiments, this is accomplished by aggregating clinical data in a system backend, and making it available via a portal. In some embodiments, this data includes not only variants and RNA expression data, but also data associated with immunotherapy markers such as MSI and TMB, as well as RNA fusions.

When the digital and laboratory health care platform further includes a device comprising a microphone and speaker for receiving audible queries or instructions from a user and delivering answers or other information, in some embodiments, the methods and systems described above are utilized to add data to a database the device can access. An example of such a device is disclosed, for example, in U.S. Patent Publication No. 2020/0335102, titled “Collaborative Artificial Intelligence Method And System,” and published Oct. 22, 2020, which is incorporated herein by reference and in its entirety for all purposes.

When the digital and laboratory health care platform further includes a mobile application for ingesting patient records, including genomic sequencing records and/or results even if they were not generated by the same digital and laboratory health care platform, in some embodiments, the methods and systems described above are utilized to receive ingested patient records. An example of such a mobile application is disclosed, for example, in U.S. Pat. No. 10,395,772, titled “Mobile Supplementation, Extraction, And Analysis Of Health Records,” and issued Aug. 27, 2019, which is incorporated herein by reference and in its entirety for all purposes. Another example of such a mobile application is disclosed, for example, in U.S. Pat. No. 10,902,952, titled “Mobile Supplementation, Extraction, And Analysis Of Health Records,” and issued Jan. 26, 2021, which is incorporated herein by reference and in its entirety for all purposes. Another example of such a mobile application is disclosed, for example, in U.S. Patent Publication No. 2021/0151192, titled “Mobile Supplementation, Extraction, And Analysis Of Health Records,” and filed May 20, 2021, which is incorporated herein by reference and in its entirety for all purposes.

When the digital and laboratory health care platform further includes organoids developed in connection with the platform (for example, from the patient specimen), in some embodiments, the methods and systems are used to further evaluate genetic sequencing data derived from an organoid and/or the organoid sensitivity, especially to therapies matched based on a portion or all of the information determined by the systems and methods, including predicted cancer type(s), likely tumor origin(s), etc. In some embodiments, these therapies are tested on the organoid, derivatives of that organoid, and/or similar organoids to determine an organoid's sensitivity to those therapies. In some embodiments, any of the results are included in a report. If the organoid is associated with a patient specimen, in some embodiments, any of the results are included in a report associated with that patient and/or delivered to the patient or patient's physician or clinician. In various examples, organoids are cultured and tested according to the systems and methods disclosed in U.S. Patent Publication No. 2021/0155989, titled “Tumor Organoid Culture Compositions, Systems, and Methods,” published May 27, 2021; PCT/US20/56930, titled “Systems and Methods for Predicting Therapeutic Sensitivity,” filed Oct. 22, 2020; U.S. Patent Publication No. 2021/0172931, titled “Large Scale Organoid Analysis,” published Jun. 10, 2021; PCT/US2020/063619, titled “Systems and Methods for High Throughput Drug Screening,” filed Dec. 7, 2020 and U.S. patent application Ser. No. 17/301,975, titled “Artificial Fluorescent Image Systems and Methods,” filed Apr. 20, 2021 which are each incorporated herein by reference and in their entirety for all purposes. In one example, the drug sensitivity assays are especially informative if the systems and methods return results that match with a variety of therapies, or multiple results (for example, multiple equally or similarly likely cancer types or tumor origins), each matching with at least one therapy.

When the digital and laboratory health care platform further includes application of one or more of the above in combination with or as part of a medical device or a laboratory developed test that is generally targeted to medical care and research, in some embodiments, such laboratory developed test or medical device results are enhanced and personalized through the use of artificial intelligence. An example of laboratory developed tests, especially those that may be enhanced by artificial intelligence, is disclosed, for example, in U.S. Patent Publication No. 2021/0118559, titled “Artificial Intelligence Assisted Precision Medicine Enhancements to Standardized Laboratory Diagnostic Testing,” and published Apr. 22, 2021, which is incorporated herein by reference and in its entirety for all purposes.

It should be understood that the examples given above are illustrative and do not limit the uses of the systems and methods described herein in combination with a digital and laboratory health care platform.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination in FIG. 1 and/or as described elsewhere within the application. These program modules can be stored on a CD-ROM, DVD, magnetic disk storage product, USB key, or any other non-transitory computer readable data or program storage product.

Many modifications and variations of this disclosure can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The disclosure is to be limited only by the terms of the appended claims, along with the full scope of equivalents to which such claims are entitled.

1. A method of determining a genotype of a subject at a genomic locus comprising a tandem repeat, from a plurality of candidate genotypes for the genomic locus, the method comprising:
at a computer system having one or more processors and memory storing at least one program for execution by the one or more processors:
(A) obtaining, in electronic form, a first set of sequence reads obtained from a biological sample of the subject that map to the tandem repeat in the genomic locus, wherein the tandem repeat consists of a plurality of contiguous nucleotide repeat units and each respective sequence read in the first set of sequence reads encompasses the tandem repeat;
(B) determining, for each respective sequence read in the first set of sequence reads, a corresponding repeat count of the number of repeat units in the plurality of contiguous repeat units in the respective sequence read, thereby determining a distribution of repeat counts of the number of repeat units in the first set of sequence reads;
(C) obtaining a plurality of sets of repeat count adjustment factors, wherein:
each respective set of repeat count adjustment factors corresponds to a candidate allele in a plurality of candidate alleles,
each respective candidate allele in the plurality of candidate alleles has a different corresponding number of repeat units for the plurality of contiguous nucleotide repeat units,
each respective set of repeat count adjustment factors includes a corresponding repeat count adjustment factor for each respective number of repeat units in a numerical range of repeat units for the plurality of contiguous nucleotide repeat units; and
each combination of two respective candidate alleles in the plurality of candidate alleles corresponds to a respective candidate genotype in the plurality of candidate genotypes;
(D) assigning, for each respective candidate genotype in the plurality of candidate genotypes, a corresponding likelihood for the respective candidate genotype based, at least in part, upon:
for each respective candidate allele corresponding to the respective candidate genotype:
(i) a proportion of sequence reads in the plurality first set of sequence reads that have the repeat count of the number of repeat units in the plurality of contiguous repeat units corresponding to the respective candidate allele; and
(ii) a repeat count adjustment factor matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles; and
(E) selecting the respective candidate genotype in the plurality of candidate genotypes having the highest corresponding likelihood.

2-3. (canceled)

4. The method of claim 1, wherein the genomic locus is a gene and wherein the gene is the UDP glucuronosyltransferase family 1 member A1 (UGT1A1) gene.

5. The method of claim 4, wherein the plurality of candidate alleles comprises a first allele comprising an A(TA)₆TAA TATA box, a second allele comprising an A(TA)₇TAA TATA box, a third allele comprising an A(TA)₈TAA TATA box, and a fourth allele comprising an A(TA)₉TAA TATA box.

6-8. (canceled)

9. The method of claim 1, wherein expansion or contraction of the tandem repeat is linked with a change in a drug metabolism.

10-13. (canceled)

14. The method of claim 1, wherein the obtaining the first set of sequence reads (A) comprises:
sequencing a first plurality of nucleic acids from the biological sample of the subject, thereby obtaining a first plurality of sequence reads that comprises the first set of sequence reads; and
mapping the first plurality of sequence reads against a genomic reference construct comprising the tandem repeat, thereby identifying a first sub-plurality of the first plurality of sequence reads that map to a genomic position within a threshold distance from the tandem repeat in the genomic reference construct.

15-17. (canceled)

18. The method of claim 14, wherein:
the obtaining the first set of sequence reads (A) further comprises aligning the first sub-plurality of the first plurality of sequence reads against a plurality of reference structures for the genomic locus, wherein each respective reference structure in the plurality of reference structures comprises a different repeat count of the number of repeat units in the tandem repeat; and
the determining (B) comprises counting, for each respective reference structure in the plurality of reference structures, a corresponding number of sequence reads in the first set of sequence reads that map to the respective reference structure.

19. The method of claim 18, wherein the plurality of reference structures is represented by a linear graph model.

20. (canceled)

21. The method of claim 14, wherein the first plurality of sequence reads is at least 100,000 sequence reads.

22. The method of claim 1, wherein the first set of sequence reads is at least 25 sequence reads.

23-26. (canceled)

27. The method of claim 1, wherein a respective repeat count adjustment factor in a respective set of repeat count adjustment factors in the plurality of sets of repeat count adjustment factors is determined based on a proportion of sequence reads, in a second set of sequence reads obtained from a reference sample, having a respective repeat count of the number of repeat units in the tandem repeat, wherein the reference sample comprises polynucleotides encompassing the tandem repeat having a known respective repeat count of the number of repeat units in the tandem repeat.

28. The method of claim 1, wherein a respective repeat count adjustment factor in a respective set of repeat count adjustment factors in the plurality of sets of repeat count adjustment factors is determined using an error model.

29. The method of claim 28, wherein the error model has the formula:
$$p ( 0 \vert 0 ) = 1 , p ( r \vert h ) = ( 1 - s ) p ( r - 1 \vert h - 1 ) + \frac{s}{2} p ( r - 2 \vert h - 1 ) + \frac{s}{2} p ( r \vert h - 1 ) ,$$
for 0≤r≤2h and 0 elsewhere, wherein:
p(r|h) is a probability of observing r repeat units in a sequence read obtained from a respective polynucleotide having h repeat units; and
s is a probability that a respective repeat unit will be duplicated or deleted during sequencing of the respective polynucleotide.

30. The method of claim 1, wherein the assigning the corresponding likelihood for the respective candidate genotype (D) is further based upon:
for each respective candidate allele in the plurality of candidate alleles that does not correspond to the respective candidate genotype:
(i) a proportion of sequence reads in the first set of sequence reads that have the repeat count of the number of repeat units in the plurality of contiguous repeat units corresponding to the respective candidate allele; and
(ii) a repeat count adjustment factor matching the number of repeat units in the plurality of contiguous repeat units corresponding to the respective candidate allele from the set of repeat count adjustment factors for the respective candidate allele.

31. The method of claim 1, wherein the corresponding likelihood for the respective candidate genotype P(H|E) is determined according to:
$$P ( H \vert E ) = \frac{P ( E \vert H ) P ( H )}{P ( E )} ,$$
wherein:
E represents the distribution of repeat counts of the number of repeat units in the first set of sequence reads,
H represents a corresponding hypothesis that the subject has the respective candidate genotype for the genomic locus,
P(H) is a prior probability that the subject has the respective candidate genotype for the genomic locus,
P(E|H) is a conditional probability of observing the distribution of repeat counts of the number of repeat units in the first set of sequence reads if the subject has the respective candidate genotype for the genomic locus, and
P(E) is a marginal probability of observing the distribution of repeat counts of the number of repeat units in the first set of sequence reads regardless of the subject's genotype for the genomic locus.

32. The method of claim 31, wherein, for each respective candidate genotype in the plurality of candidate genotypes, the conditional probability is:
$$P ( E \vert H ) = {\prod }_{r} {P ( r \vert H )}^{{f}_{r}} ,$$
wherein:
P(r|H) is the probability of observing r repeat units in a respective sequence read if the subject has the respective candidate genotype for the genomic locus determined by:
$$P ( r \vert H ) = \{ \begin{matrix}\frac{1}{2} [ p ( r \vert a ) + p ( r \vert b ) ] & if a \neq b ( heterozygous ) \\ p ( r \vert a ) & if a = b ( homozygous ) ,\end{matrix}$$
wherein:
when the candidate genotype for the genomic locus is a homozygous genotype for a candidate allele in the plurality of candidate alleles, P(r|H) is the repeat count adjustment factor, in the set of repeat count adjustment factors corresponding to the candidate allele, corresponding to r repeat units, and
when the candidate genotype for the genomic locus is a heterozygous genotype for a first candidate allele in the plurality of candidate alleles and a second candidate allele in the plurality of candidate alleles, P(r|H) is an arithmetic combination of (i) the repeat count adjustment factor, in the set of repeat count adjustment factors corresponding to the first candidate allele, corresponding to r repeat units, and (ii) the repeat count adjustment factor, in the set of repeat count adjustment factors corresponding to the second candidate allele, corresponding to r repeat units, and
fᵣ is a corresponding count of sequence reads, in the first plurality set of sequence reads, having r repeat units.

33. (canceled)

34. The method of claim 1, wherein the assigning the corresponding likelihood for the respective candidate genotype (D) further comprises determining a corresponding first quality metric for the respective candidate genotype.

35. The method of claim 34, wherein the corresponding first quality metric for the respective candidate genotype is a log-odds ratio of the corresponding likelihood for the respective candidate genotype.

36. The method of claim 34, further comprising filtering the plurality of candidate genotypes based on the corresponding first quality metric by a procedure comprising:
when the corresponding first quality metric satisfies a threshold quality metric score, retaining the respective candidate genotype in the plurality of candidate genotypes; and
when the corresponding first quality metric fails to satisfy the threshold quality metric score, removing the respective candidate genotype from the plurality of candidate genotypes.

37. The method of claim 34, further comprising generating a report comprising at least (i) the respective candidate genotype in the plurality of candidate genotypes having the highest corresponding likelihood and (ii) the corresponding first quality metric for the respective candidate genotype in the plurality of candidate genotypes having the highest corresponding likelihood.

38-46. (canceled)

47. A computer system comprising:
one or more processors;
memory; and
one or more programs, wherein the one or more programs are stored in the memory and are configured to be executed by the one or more processors, the one or more programs including instructions for determining a genotype of a subject at a genomic locus comprising a tandem repeat, from a plurality of candidate genotypes for the genomic locus, by a method comprising:
(A) obtaining, in electronic form, a first set of sequence reads obtained from a biological sample of the subject that map to the tandem repeat in the genomic locus, wherein the tandem repeat consists of a plurality of contiguous nucleotide repeat units and each respective sequence read in the first set of sequence reads encompasses the tandem repeat;
(B) determining, for each respective sequence read in the first set of sequence reads, a corresponding repeat count of the number of repeat units in the plurality of contiguous repeat units in the respective sequence read, thereby determining a distribution of repeat counts of the number of repeat units in the first set of sequence reads;
(C) obtaining a plurality of sets of repeat count adjustment factors, wherein:
each respective set of repeat count adjustment factors corresponds to a candidate allele in a plurality of candidate alleles,
each respective candidate allele in the plurality of candidate alleles has a different corresponding number of repeat units for the plurality of contiguous nucleotide repeat units,
each respective set of repeat count adjustment factors includes a corresponding repeat count adjustment factor for each respective number of repeat units in a numerical range of repeat units for the plurality of contiguous nucleotide repeat units; and
each combination of two respective candidate alleles in the plurality of candidate alleles corresponds to a respective candidate genotype in the plurality of candidate genotypes;
(D) assigning, for each respective candidate genotype in the plurality of candidate genotypes, a corresponding likelihood for the respective candidate genotype based, at least in part, upon:
for each respective candidate allele corresponding to the respective candidate genotype:
(i) a proportion of sequence reads in the plurality first set of sequence reads that have the repeat count of the number of repeat units in the plurality of contiguous repeat units corresponding to the respective candidate allele; and
(ii) a repeat count adjustment factor matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles; and
(E) selecting the respective candidate genotype in the plurality of candidate genotypes having the highest corresponding likelihood.

48. A computer readable storage medium storing one or more programs, the one or more programs comprising instructions that, when executed by an electronic device with one or more processors and a memory, cause the electronic device to perform a method for determining a genotype of a subject at a genomic locus comprising a tandem repeat, from a plurality of candidate genotypes for the genomic locus, comprising:
(A) obtaining, in electronic form, a first set of sequence reads obtained from a biological sample of the subject that map to the tandem repeat in the genomic locus, wherein the tandem repeat consists of a plurality of contiguous nucleotide repeat units and each respective sequence read in the first set of sequence reads encompasses the tandem repeat;
(B) determining, for each respective sequence read in the first set of sequence reads, a corresponding repeat count of the number of repeat units in the plurality of contiguous repeat units in the respective sequence read, thereby determining a distribution of repeat counts of the number of repeat units in the first set of sequence reads;
(C) obtaining a plurality of sets of repeat count adjustment factors, wherein:
each respective set of repeat count adjustment factors corresponds to a candidate allele in a plurality of candidate alleles,
each respective candidate allele in the plurality of candidate alleles has a different corresponding number of repeat units for the plurality of contiguous nucleotide repeat units,
each respective set of repeat count adjustment factors includes a corresponding repeat count adjustment factor for each respective number of repeat units in a numerical range of repeat units for the plurality of contiguous nucleotide repeat units; and
each combination of two respective candidate alleles in the plurality of candidate alleles corresponds to a respective candidate genotype in the plurality of candidate genotypes;
(D) assigning, for each respective candidate genotype in the plurality of candidate genotypes, a corresponding likelihood for the respective candidate genotype based, at least in part, upon:
for each respective candidate allele corresponding to the respective candidate genotype:
(i) a proportion of sequence reads in the first set of sequence reads that have the repeat count of the number of repeat units in the plurality of contiguous repeat units corresponding to the respective candidate allele; and
(ii) a repeat count adjustment factor matching the number of repeat units in the plurality of contiguous repeat units corresponding to the respective candidate allele from the set of repeat count adjustment factors for the respective candidate allele, thereby generating a corresponding first likelihood for each respective candidate allele in the plurality of candidate alleles; and
(E) selecting the respective candidate genotype in the plurality of candidate genotypes having the highest corresponding likelihood.
